(19) United States
(12) Patent Application Publication
(10) Pub. No.: US 2014/0309413 A1
(43) Pub. Date: Oct. 16, 2014

(54) METHODS OF STEREOSELECTIVE SYNTHESIS OF SUBSTITUTED NUCLEOSIDE ANALOGS

(71) Applicant: Vertex Pharmaceuticals Incorporated, Boston, MA (US)

(72) Inventors: Peter Jamison Rose, Littleton, MA (US); Young Chun Jung, Lexington, MA (US); Cavan McKeon Bligh, Somerville, MA (US); Shereen Ibrahim, Cambridge, MA (US); Luigi Anzalone, West Chester, PA (US); David B. Miller, JR., Waltham, MA (US); John Gregg Van Alsten, Niantic, CT (US); Timothy Thomas Curran, Milford, MA (US)

(73) Assignee: Vertex Pharmaceuticals Incorporated, Boston, MA (US)

(21) Appl. No.: 14/203,332

(22) Filed: Mar. 10, 2014

Related U.S. Application Data
(63) Continuation-in-part of application No. PCT/US2013/030285, filed on Mar. 11, 2013.
(60) Provisional application No. 61/877,362, filed on Sep. 13, 2013.

(51) Int. Cl. C07H 1/02; C07F 9/58; C07H 19/10

(52) U.S. Cl. CPC C07H 1/02; C07H 19/10; C07F 9/585
USPC 536/26.8; 546/25

(57) ABSTRACT

The present invention relates to the novel diastereoselective syntheses for generating phosphorothioate compounds. Examples include nucleoside phosphorothioate analogs that are useful in treating diseases and/or conditions such as viral infections.

### CROSS REFERENCE TO RELATED APPLICATION

This PCT application claims the benefit of PCT Application Serial No. PCT/US2013/030285, filed on Mar. 11, 2013, and U.S. provisional application Ser. No. 61/877,362, filed on Sep. 13, 2013. Both of these documents are hereby incorporated by reference in their entireties.

### FIELD OF THE INVENTION

The present application relates to the fields of synthetic organic chemistry, biochemistry, and medicine. Disclosed herein are methods of generating phosphorothioate compounds (e.g., phosphorothioate nucleoside analogs), including diastereoselective syntheses.

### BACKGROUND OF THE INVENTION

Phosphorothioate compounds possess a variety of known uses. For example, insecticides such as Diazinon, Parathion and Malathion contain phosphorothioate functionalities in their chemical structures. Compounds containing monothiophosphate esters are often used as biological probes in various biochemical assays. And, nucleoside analogs are a class of compounds that have been shown to exert antiviral and anticancer activity both in vitro and in vivo, and thus, have been the subject of widespread research for the treatment of viral infections and cancer.

Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY OF THE INVENTION

The present application relates to processes and intermediates that are useful for generating phosphorothioate compounds.

In one aspect, this application provides a method of preparing a compound of Formula I:

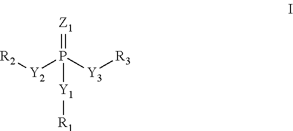

or pharmaceutically acceptable salt thereof wherein Z₁ is O or S; each of Y₁, Y₂ and Y₃ is independently a bond, —S—, —O—, or —NR₁₀₀—, R₁₀₀ is hydrogen, C₁₋₆ alkyl, C₂₋₆ alkenyl, C₂₋₆ alkynyl, aryl, heteroaryl, aryl(C₁₋₆ alkyl), C₃₋₈ cycloaliphatic, or a saturated, partially unsaturated, or fully unsaturated 3-8 membered heterocyclic ring having up to 3 heteroatoms independently selected from N, O, or S; and each of R₁, R₂ and R₃ is independently -L-R₅, wherein each L is independently a bond, —(CH₂)ₘ—, —(CH₂)ₘ, —(CHR₆)ₚ—, —(CH₂)ₘ, —(CR₆R₇)ₚ—, or —(C(R₈)₂)ₘC(O)O—, each of R₆ and R₇ is independently selected from hydrogen, halogen, —OH, —N(R₈)₂, or —OR₈, each R₈ is independently hydrogen or C₁₋₆ alkyl, each m is independently 0-3, each p is independently 0-3, each R₅ is independently hydrogen, —O⁻, —OH, alkoxy, C₁₋₆ alkyl, C₂₋₆ alkenyl, C₂₋₆ alkynyl, —(C(R₈)₂)ₘC(O)OR₈, aryl, aryl(C₁₋₆ alkyl), C₃₋₈ cycloaliphatic, heteroaryl, or a saturated or partially unsaturated 3-8 membered heterocyclic ring having up to 3 heteroatoms independently selected from N, O, or S, an optionally substituted amine, an optionally substituted N-linked amino acid, an optionally substituted N-amino acid ester derivative, or

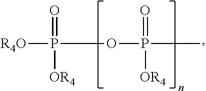

wherein each R₄ is independently absent or hydrogen, and n is 0 or 1, and wherein the alkyl, alkenyl, alkynyl, aryl, aryl-(C₁₋₆ alkyl), cycloaliphatic, heteroaryl, or heterocyclic ring groups are each optionally substituted with 1-3 groups independently selected from halo, —OH, —CN, azido, optionally substituted C₁₋₆ alkyl, optionally substituted C₁₋₆ alkoxy, an optionally substituted heterocyclic base, or an optionally substituted heterocyclic base with a protected amino group; comprising i) reacting a compound of Formula A with a compound of Formula B

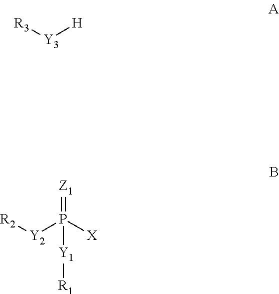

wherein X is a leaving group, in the presence of an acid or a metal salt, to generate the compound of Formula I.

In some methods, Y₁ is a bond; each of Y₂ and Y₃ is independently —O—, or —S—; R₁ is —O⁻, —OH, alkoxy, an optionally substituted amine, an optionally substituted N-linked amino acid or an optionally substituted N-amino acid ester derivative; and each of R₂ and R₃ is independently hydrogen, C₁₋₆ alkyl, aryl, heteroaryl, aryl(C₁₋₆ alkyl), or C₃₋₈ cycloaliphatic.

In some methods, R₁₀₀ is hydrogen or C₁₋₆ alkyl. For example, R₁₀₀ is selected from hydrogen, methyl, or ethyl.

In some methods, —Y₁—R₁ is an optionally substituted N-linked amino acid or an optionally substituted N-amino acid ester derivative; and R₂ is optionally substituted aryl. For example, —Y₁—R₁ is

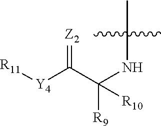

wherein Z₂ is O or S; Y₄ is a bond, —S—, —O—, or —NR₁₀₀—; each of R₉ and R₁₀ is independently selected from hydrogen, C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl, or R₉ and R₁₀ taken together with the carbon atom to which they are attached form a C₃₋₆ cycloalkyl; and R₁₁ is hydrogen, C₁₋₆ alkyl, C₃₋₈ cycloalkyl, aryl, aryl(C₁₋₆ alkyl), or halo-C₁₋₆ alkyl.

In some methods, R₂ is optionally substituted aryl. For example, R₂ is unsubstituted phenyl.

In some methods, the reaction of step i) occurs in the presence of an acid. For example, the reaction of step i) occurs in the presence of an acid, and the acid is a strong organic acid. In some examples, the reaction of step i) occurs in the presence of a sulfonic acid (e.g., trifluoromethanesulfonic acid or methanesulfonic acid).

In some methods, the reaction of step i) occurs in the presence of a salt. In some examples, the metal salt is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, or any combination thereof. For example, the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

In other methods, X is —W—R₁₂; W is a bond, —S—, or —O—; and R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl, or —W—R₁₂ is

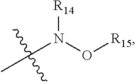

wherein each of R₁₄ and R₁₅ is independently C₁₋₆ alkyl, cycloalkyl, or heteroalkyl, or R₁₄ and R₁₅, taken together with the heteroatoms to which they are attached, form a 6-10-membered heterocyclic ring optionally substituted with 1-3 of R₁₃.

In some methods, W is —S— or —O—; and R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, —W—R₁₂ is selected from

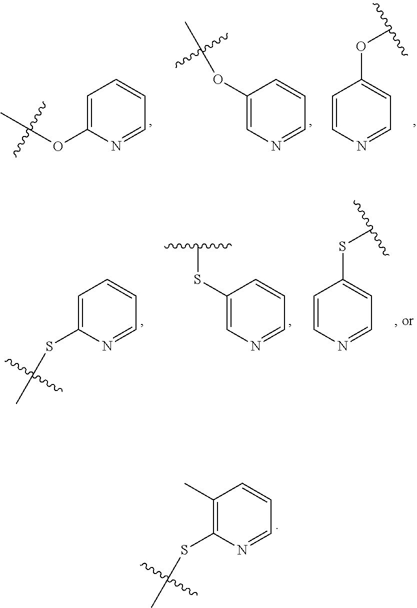

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

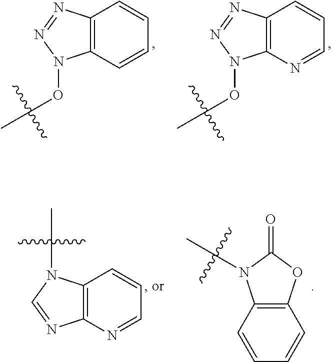 [SMILES: *On1nnc2ccccc21 ; *On1nnc2cccnc21 ; *n1cnc2ncccc21 ; *n1c(=O)oc2ccccc21]

In some methods, —W—R₁₂ is

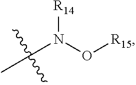

wherein each of R₁₄ and R₁₅ is independently C₁₋₆ alkyl, cycloalkyl, or heteroalkyl, or R₁₄ and R₁₅ taken together with the heteroatoms to which they are attached form a 6-10 membered heterocyclic ring optionally substituted with 1-3 of R₁₃. For example, —W—R₁₂ is selected from

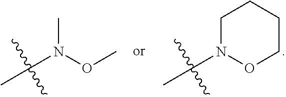

In some methods, R₁₂ is a 5-6-membered fully saturated or partially unsaturated heterocyclic ring having 1-2 heteroatoms independently selected from N, O, or S, optionally substituted with 1-3 of R₁₃. For example, —W—R₁₂ is

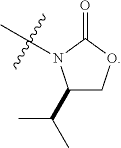 [SMILES: *N1C(=O)OC[C@H]1C(C)C]

In some methods, the compound of Formula B is a compound of Formula B-2a or B-2b:

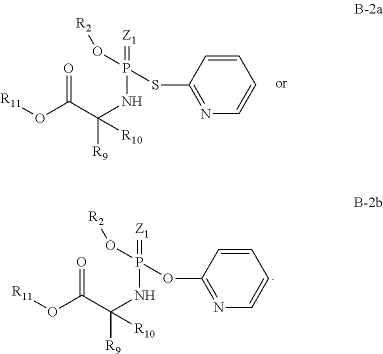

In some methods, the reaction of step i) occurs in the presence of an organic solvent. In some examples, the organic solvent of step i) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step i) is performed at a temperature of about 30° C. or less. For example, the reaction of step i) is performed at a temperature of from about −20° C. to about 25° C.

Some methods further comprise step ii): reacting a compound of Formula B-3, wherein X^{A} is halogen, with H—W—R₁₂

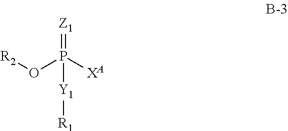

in the presence of a base to generate the compound of Formula B-1

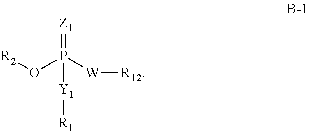

In some methods, the base of step ii) is an amine base. For example, the base of step ii) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step ii) is performed in the presence of an organic solvent. For example, the organic solvent of step ii) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step ii) is performed at a temperature of about 30° C. or less. For example, the reaction of step ii) is performed at a temperature of from about −10° C. to about 25° C.

In some methods, the compound of Formula B-3 is a compound of Formula B-4, wherein X^{A} is halogen:

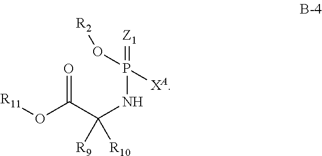

Some methods further comprise step iii): reacting a compound of Formula B-5, wherein X^{B} is halogen, with a compound of Formula C:

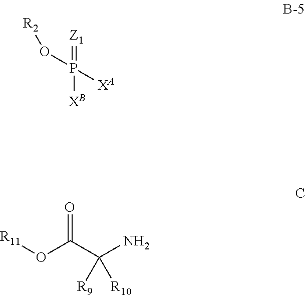

under nucleophilic substitution conditions to generate the compound of Formula B-4.

Another aspect of this application provides a method of preparing a compound of Formula II:

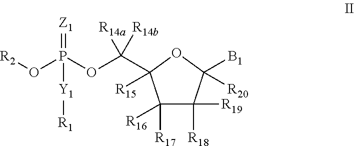

or a pharmaceutically acceptable salt thereof; wherein Z₁ is S or O; B₁ is an optionally substituted heterocyclic base or an optionally substituted heterocyclic base with a protected amino group; —Y₁—R₁ is —O⁻, —OH, alkoxy, an optionally substituted amine, an optionally substituted N-linked amino acid or an optionally substituted N-amino acid ester derivative; R₂ is an optionally substituted aryl, an optionally substituted heteroaryl, an optionally substituted heterocyclyl or

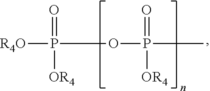

wherein each R₄ is independently absent or hydrogen, and n is 0 or 1; each of R₁₄ₐ and R_{14b} is independently selected from hydrogen, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or R₁₄ₐ and R_{14b} taken together with the carbon atom to which they are attached form an optionally substituted C₃₋₆ cycloalkyl; R₁₅ is hydrogen, azido, an optionally substituted C₁₋₆ alkyl, an optionally substituted C₂₋₆ alkenyl, or an optionally substituted C₂₋₆ alkynyl; each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or R₁₇ and R₁₈ are both oxygen atoms that are linked together by —(CR₂₁R₂₂)— or by a carbonyl group; R₂₀ is hydrogen, halogen, azido, cyano, an optionally substituted C₁₋₆ alkyl, or —OR₂₁; and each of R₂₁ and R₂₂ is independently selected from hydrogen, optionally substituted C₁₋₆ alkyl or optionally substituted C₃₋₆ cycloalkyl; comprising the step ia): reacting a compound of Formula A-1 with a compound of Formula B-X

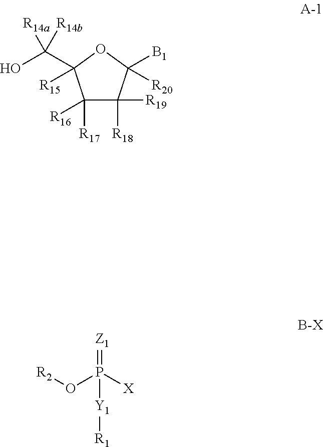

wherein X is a leaving group, in the presence of an acid or a metal salt, to generate the compound of Formula II.

In some methods, B₁ is an optionally substituted saturated or partially unsaturated 5-7-membered monocyclic heterocycle having at least 1 nitrogen atom and 0 to 2 additional heteroatoms independently selected from N, O, or S; or B₁ is an optionally substituted saturated or partially unsaturated 8-10-membered bicyclic heterocycle having at least 2 nitrogen atoms and 0 to 3 additional heteroatoms independently selected from N, O, or S. For example, B₁ is selected from

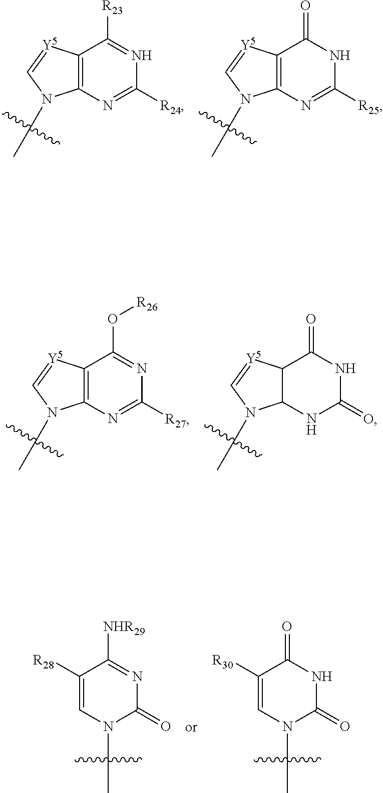

wherein Y⁵ is ═N— or ═CR₃₁—, wherein R₃₁ is C₁₋₆ alkyl, or C₂₋₆ alkenyl; R₂₃ is halogen or —NHR₃₂, wherein R₃₂ is hydrogen, C₁₋₆ alkyl, C₂₋₆ alkenyl, C₃₋₈ cycloalkyl, —O—C₁₋₆ alkyl, —C(O)R^{A}, or —C(O)OR^{A}; R₂₄ is hydrogen, halogen, or —NHR₃₃; R₂₅ is hydrogen or —NHR₃₃; R₂₆ is hydrogen, halogen, C₁₋₆ alkyl, or C₂₋₆ alkenyl; R₂₇ is hydrogen, C₁₋₆ alkyl, C₃₋₈ cycloalkyl, —C(O)R^{A}, or —C(O)OR^{A}; R₂₈ is hydrogen, halogen, C₁₋₆ alkyl, or C₂₋₆ alkenyl; R₂₉ is hydrogen, halogen, C₁₋₆ alkyl, or C₂₋₆ alkenyl; R₃₀ is hydrogen, halogen, —NHR₃₃, C₁₋₆ alkyl, or C₂₋₆ alkenyl; each R₃₃ is independently selected from hydrogen, —C(O)R^{A}, or —C(O)OR^{A}; and each R^{A} is independently selected from C₁₋₆ alkyl, C₂₋₆ alkenyl, C₃₋₈ cycloalkyl, aryl, heteroaryl, heterocyclyl, aryl(C₁₋₆ alkyl), heteroaryl(C₁₋₆ alkyl), or heterocyclyl(C₁₋₆ alkyl). In other examples, B₁ is selected from

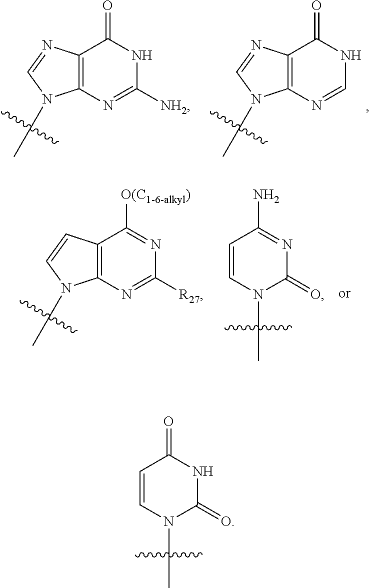

In some methods, —Y₁—R₁ is

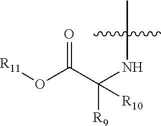

wherein each of R₉ and R₁₀ is independently selected from hydrogen, C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl, or R₉ and R₁₀ taken together with the carbon atom to which they are attached form a C₃₋₆ cycloalkyl; and R₁₁ is hydrogen, C₁₋₆ alkyl, C₃₋₈ cycloalkyl, aryl, aryl(C₁₋₆ alkyl), or halo-C₁₋₆ alkyl.

In some methods, R₂ is optionally substituted aryl or optionally substituted heteroaryl. For example, R₂ is optionally substituted aryl. In other examples, R₂ is unsubstituted phenyl.

In some methods, the reaction of step ia) occurs in the presence of an acid. For example, the reaction of step ia) occurs in the presence of an acid, and the acid is a strong organic acid. In some examples, the reaction of step ia) occurs in the presence of a sulfonic acid (e.g., trifluoromethanesulfonic acid or methanesulfonic acid).

In some methods, the reaction of step ia) occurs in the presence of a salt. In some examples, the metal salt (e.g., the alkali metal salt or the transition metal salt) is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, or any combination thereof. For example, the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

In some methods, the compound of Formula B-X is a compound of Formula B-1:

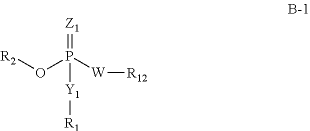

wherein W is a bond, —S—, or —O—; and R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl, or —W—R₁₂ is

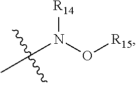

wherein each of R₁₄ and R₁₅ is independently C₁₋₆ alkyl, cycloalkyl, or heteroalkyl, or R₁₄ and R₁₅, taken together with the heteroatoms to which they are attached, form a 6-10-membered heterocyclic ring optionally substituted with 1-3 of R₁₃.

In some methods, W is —S— or —O—; and R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, —W—R₁₂ is selected from

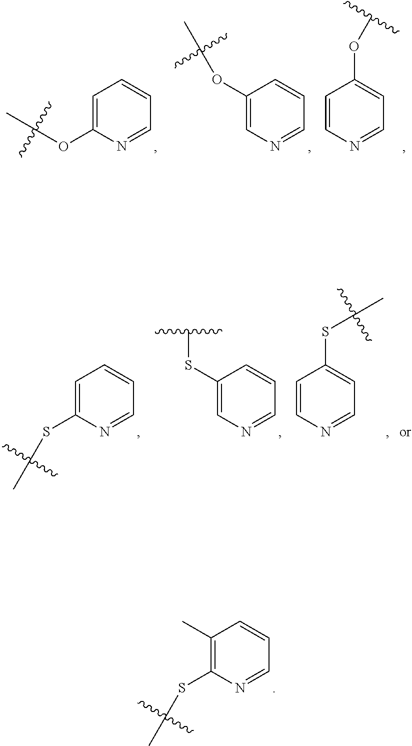

In some methods, R₁₂ is a 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

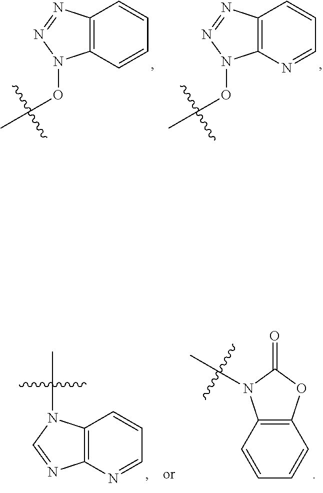

In some methods, —W—R₁₂ is

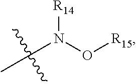

wherein R₁₄ and R₁₅ are each independently C₁₋₆ alkyl, cycloalkyl, or heteroalkyl, or R₁₄ and R₁₅ taken together with the heteroatoms to which they are attached form a 6-10-membered heterocyclic ring optionally substituted with 1-3 of R₁₃. For example, —W—R₁₂ is selected from

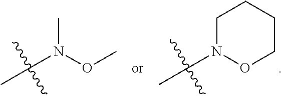

In some methods, the compound of Formula B-1 is a compound of Formula B-2a or B-2b:

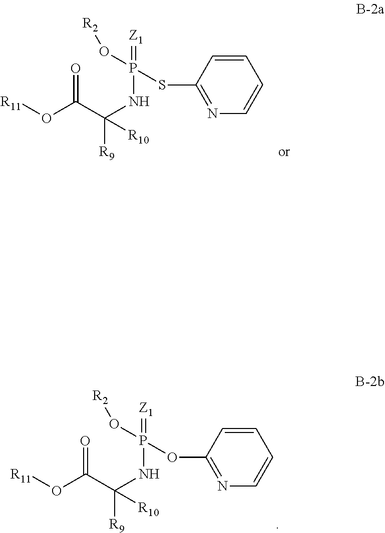

In some methods, the reaction of step ia) occurs in the presence of an organic solvent. In some examples, the organic solvent of step ia) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ia) is performed at a temperature of about 30° C. or less. For example, the reaction of step ia) is performed at a temperature of from about −20° C. to about 25° C.

Some methods further comprise step ii): reacting a compound of Formula B-3, wherein X^{A} is halogen, with H—W—R₁₂

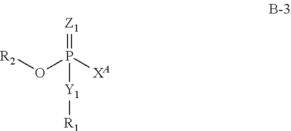

in the presence of a base to generate the compound of Formula B-1.

In some methods, the base of step ii) is an amine base. For example, the base of step ii) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step ii) is performed in the presence of an organic solvent. For example, the organic solvent of step ii) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step ii) is performed at a temperature of about 30° C. or less. For example, the reaction of step ii) is performed at a temperature of from about −10° C. to about 25° C.

In some methods, the compound of Formula B-3 is a compound of Formula B-4, wherein X^{A} is halogen:

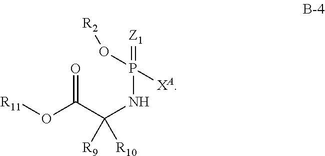

Some methods further comprise step iii): reacting a compound of Formula B-5, wherein X^{B} is halogen, with a compound of Formula C:

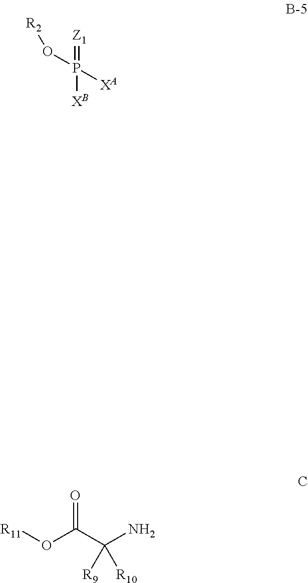

under nucleophilic substitution conditions to generate the compound of Formula B-4.

Another aspect of this application provides a method of preparing a compound of Formula III:

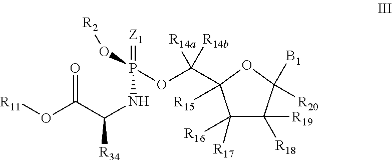

or a pharmaceutically acceptable salt thereof, having a diastereomeric purity of about 75% or greater, wherein B₁ is an optionally substituted heterocyclic base or an optionally substituted heterocyclic base with a protected amino group; Z₁ is S or O; R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; R₂ is an optionally substituted aryl, an optionally substituted heteroaryl, an optionally substituted heterocyclyl or

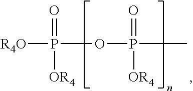

wherein each R₄ is independently absent or hydrogen, and n is 0 or 1; each of R₁₄ₐ and R_{14b} is independently selected from hydrogen, deuterium, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or R₁₄ₐ and R_{14b} taken together with the carbon atom to which they are attached form an optionally substituted C₃₋₆ cycloalkyl; R₁₅ is hydrogen, azido, an optionally substituted C₁₋₆ alkyl, an optionally substituted C₂₋₆ alkenyl, or an optionally substituted C₂₋₆ alkynyl; each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or R₁₇ and R₁₈ are both oxygen atoms that are linked together by —(CR₂₁R₂₂)— or by a carbonyl group; R₂₀ is hydrogen, halogen, azido, cyano, an optionally substituted C₁₋₆ alkyl, or —OR₂₁; and each of R₂₁ and R₂₂ is independently selected from hydrogen, optionally substituted C₁₋₆ alkyl or optionally substituted C₃₋₆ cycloalkyl; comprising the step ib): reacting a compound of Formula A-1 and a compound of Formula B-1B

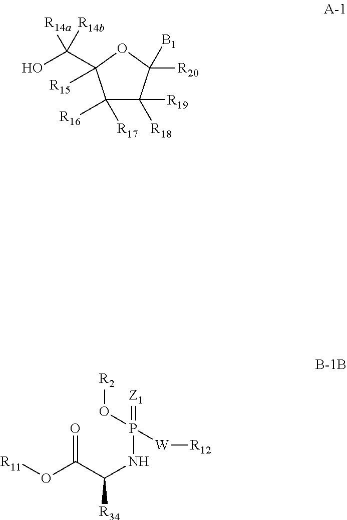

in the presence of an acid or a metal salt, wherein W is a bond, —S—, or —O—; and R₁₂ is a 5-10-membered mono- or bicyclic saturated, partially unsaturated, or fully unsaturated heterocyclic ring having 1-4 heteroatoms independently selected from N, O, or S, wherein R₁₂ is optionally substituted with 1-2 of C₁₋₆ alkyl to generate the compound of Formula III.

In some methods, B₁ is an optionally substituted saturated or partially unsaturated 5-7-membered monocyclic heterocycle having at least 1 nitrogen atom and 0 to 2 additional heteroatoms independently selected from N, O, or S; or an optionally substituted saturated or partially unsaturated 8-10-membered bicyclic heterocycle having at least 2 nitrogen atoms and 0 to 3 additional heteroatoms independently selected from N, O, or S. For example, B₁ is selected from

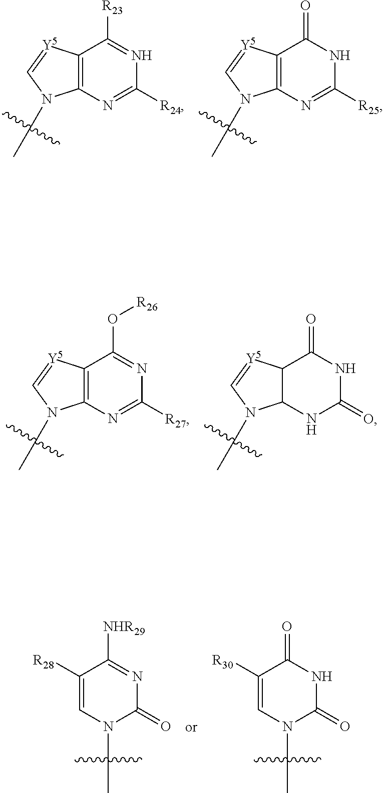

wherein Y⁵ is ═N— or ═CR₃₁—, wherein R₃₁ is C₁₋₆ alkyl, or C₂₋₆ alkenyl; R₂₃ is halogen or —NHR₃₂, wherein R₃₂ is hydrogen, C₁₋₆ alkyl, C₂₋₆ alkenyl, C₃₋₈ cycloalkyl, —O—C₁₋₆ alkyl, —C(O)R^{A}, or —C(O)OR^{A}; R₂₄ is hydrogen, halogen, or —NHR₃₃; R₂₅ is hydrogen or —NHR₃₃; R₂₆ is hydrogen, halogen, C₁₋₆ alkyl, or C₂₋₆ alkenyl; R₂₇ is hydrogen, C₁₋₆ alkyl, C₃₋₈ cycloalkyl, —C(O)R^{A}, or —C(O)OR^{A}; R₂₈ is hydrogen, halogen, C₁₋₆ alkyl, or C₂₋₆ alkenyl; R₂₉ is hydrogen, halogen, C₁₋₆ alkyl, or C₂₋₆ alkenyl; R₃₀ is hydrogen, halogen, —NHR₃₃, C₁₋₆ alkyl, or C₂₋₆ alkenyl; each R₃₃ is independently selected from hydrogen, —C(O)R^{A}, or —C(O)OR^{A}; and each R^{A} is independently selected from C₁₋₆ alkyl, C₂₋₆ alkenyl, C₃₋₈ cycloalkyl, aryl, heteroaryl, heterocyclyl, aryl(C₁₋₆ alkyl), heteroaryl(C₁₋₆ alkyl), or heterocyclyl(C₁₋₆ alkyl). In other examples, B₁ is selected from

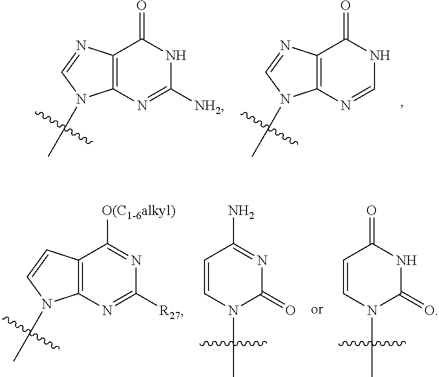

In some methods, R₂ is optionally substituted aryl or optionally substituted heteroaryl. For example, R₂ is optionally substituted aryl. In other examples, R₂ is unsubstituted phenyl.

In some methods, W is —S— or —O—; and R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, —W—R₁₂ is selected from

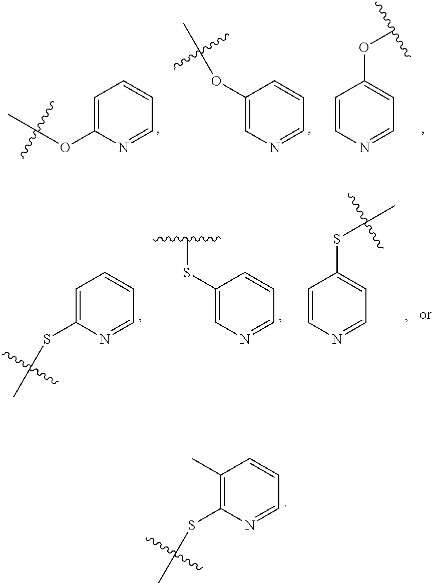

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

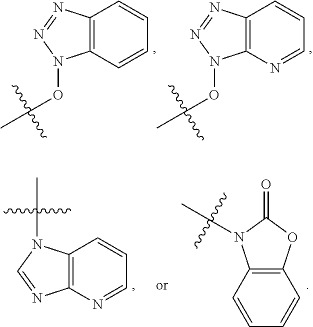 [SMILES: *On1nnc2ccccc21 ; *On1nnc2cccnc21 ; *n1cnc2ncccc21 ; *n1c(=O)oc2ccccc21]

In some methods, the reaction of step ib) is performed in the presence of a strong acid. For example, the acid of step ib) is a sulfonic acid (e.g., trifluoromethanesulfonic acid or methanesulfonic acid).

In some methods, the reaction of step ib) occurs in the presence of a salt. In some examples, the metal salt is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, or any combination thereof. For example, the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

In some methods, the reaction of step ia) occurs in the presence of an organic solvent. In some examples, the organic solvent of step ia) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ia) is performed at a temperature of about 30° C. or less. For example, the reaction of step ia) is performed at a temperature of from about −20° C. to about 25° C.

Some methods further comprise step iib): reacting a compound of Formula C-1, wherein X^{A} is halogen, with H—W—R₁₂

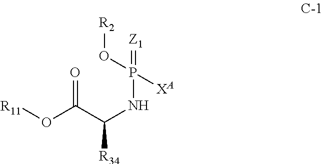

in the presence of a base to generate the compound of Formula B-1B.

In some methods, the base of step iib) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iib) is performed in the presence of an organic solvent. For example, the organic solvent of step iib) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iib) is performed at a temperature of about 30° C. or less. For example, the reaction of step iib) is performed at a temperature of from about −10° C. to about 25° C.

Some methods further comprise step iiib): reacting a compound of Formula B-5, wherein X^{B} is halogen, with a compound of Formula C-2

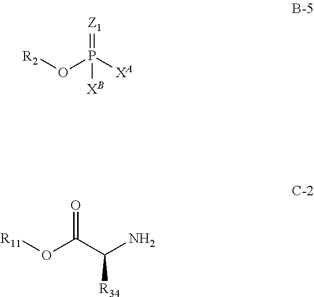

under nucleophilic substitution conditions to generate the compound of Formula C-1.

Another aspect of this application provides a method of preparing a compound of Formula IV

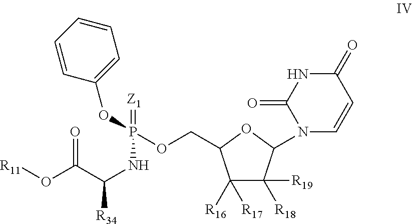

or a pharmaceutically acceptable salt thereof, having a diastereomeric purity of about 75% or greater (e.g., about 80% or greater), wherein Z is S or O; R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; each of R₁₆, R₁₇, R₁₈, and R₁₉ is independently selected from hydrogen, —OH, halogen, azido, cyano, an optionally substituted C₁₋₆ alkyl, —OR₂₀ or —OC(O)R₂₁, or R₁₇ and R₁₈ are both oxygen atoms that are linked together by —(CR₂₁R₂₂)— or by a carbonyl group; and each of R₂₀, R₂₁, and R₂₂ is independently selected from hydrogen, optionally substituted C₁₋₆ alkyl or optionally substituted C₃₋₆ cycloalkyl; comprising the step ic): reacting a compound of Formula A-2 with a compound of Formula B-1C

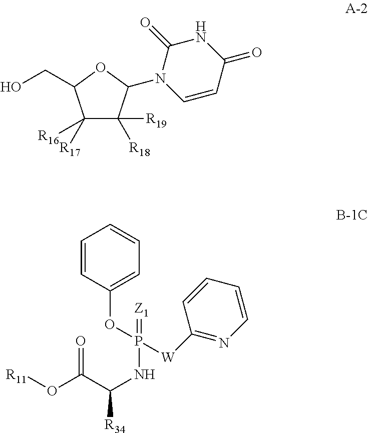

wherein W is —S— or —O—, in the presence of an acid or a metal salt to generate the compound of Formula IV.

In some methods, the reaction of step ic) is performed in the presence of a strong acid. For example, the acid of step ic) is a sulfonic acid (e.g., trifluoromethanesulfonic acid or methanesulfonic acid).

In some methods, the reaction of step ic) occurs in the presence of a salt. In some examples, the metal salt is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, or any combination thereof. For example, the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

In some methods, the reaction of step ic) occurs in the presence of an organic solvent. In some examples, the organic solvent of step ic) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ic) occurs in the presence of a mixture of solvents comprising a halogenated organic solvent and an aromatic hydrocarbon in 1:5 ratio. In one such example, the mixture of solvents comprises dichloromethane and toluene.

In other methods, the reaction of step ic) occurs in the presence of a mixture of solvents in the ratios of 1:1 to 4:1. In one such example, the mixture of solvents comprises dichloromethane and 1,4-dioxane.

In another example, the reaction of step ic) occurs in the presence of a mixture of solvents comprising dichloromethane and sulfolane in 1:1 ratio.

In some methods, the reaction of step ic) is performed at a temperature of about 30° C. or less. For example, the reaction of step ic) is performed at a temperature of from about −20° C. to about 25° C.

In some methods, the compound of Formula B-1C is a compound of Formula B-4B1 or B-4B2:

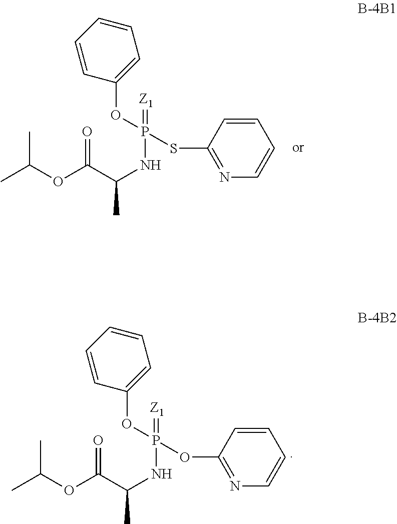

Some methods further comprise step iic): reacting a compound of Formula C-3, wherein X^{A} is halogen,

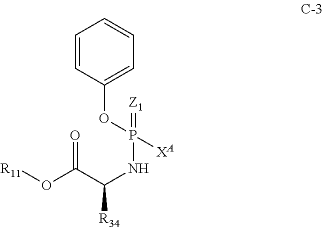

with

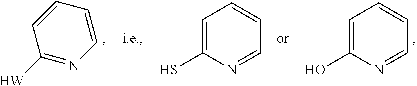

in the presence of a base to generate the compound of Formula B-1C.

In some methods, the base of step iic) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iic) is performed in the presence of an organic solvent. For example, the organic solvent of step iic) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iic) is performed at a temperature of about 30° C. or less. For example, the reaction of step iic) is performed at a temperature of from about −10° C. to about 25° C.

Some methods further comprise step iiic): reacting a compound of Formula B-5B, wherein X^{B} is halogen, with a compound of Formula C-2

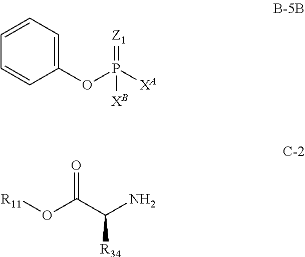

under nucleophilic substitution conditions to generate the compound of Formula C-3.

Another aspect of this application provides a method of preparing a compound of Formula V

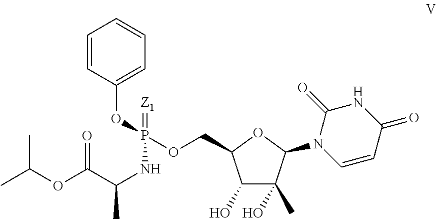

or a pharmaceutically acceptable salt thereof, having a diastereomeric purity of about 75% or greater; wherein Z₁ is S or O, comprising the step id): reacting a compound of Formula A-3 and a compound of Formula B-4B1

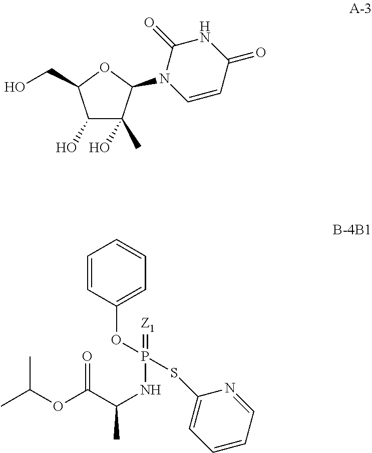

in the presence of an acid or a metal salt to generate the compound of Formula V.

In some methods, the reaction of step id) is performed in the presence of a strong acid. For example, the acid of step id) is a sulfonic acid (e.g., trifluoromethanesulfonic acid or methanesulfonic acid).

In some methods, the reaction of step id) occurs in the presence of a salt. In some examples, the metal salt is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, or any combination thereof. For example, the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

In some methods, the reaction of step id) occurs in the presence of an organic solvent. In some examples, the organic solvent of step id) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step id) occurs in the presence of a mixture of solvents comprising a halogenated organic solvent and an aromatic hydrocarbon in 1:5 ratio. In one such example, the mixture of solvents comprises dichloromethane and toluene.

In other methods, the reaction of step id) occurs in the presence of a mixture of solvents in the ratios of 1:1 to 4:1. In one such example, the mixture of solvents comprises dichloromethane and 1,4-dioxane.

In another example, the reaction of step id) occurs in the presence of a mixture of solvents comprising dichloromethane and sulfolane in 1:1 ratio.

In some methods, the reaction of step id) is performed at a temperature of about 30° C. or less. For example, the reaction of step id) is performed at a temperature of from about −20° C. to about 25° C.

Another aspect of this application provides a method of preparing a compound of Formula B-1B:

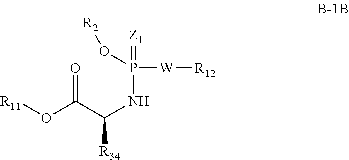

wherein Z₁ is S or O; R₂ is optionally substituted aryl or optionally substituted heteroaryl; W is a bond, —S—, or —O—; and R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl, or —W—R₁₂ is

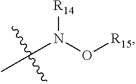

wherein each of R₁₄ and R₁₅ is independently C₁₋₆ alkyl, cycloalkyl, or heteroalkyl, or R₁₄ and R₁₅, taken together with the heteroatoms to which they are attached, form a 6-10-membered heterocyclic ring optionally substituted with 1-3 of R₁₃; R₃₄ is C₁₋₆ alkyl, halo-C₁₋₆ alkyl, C₃₋₈ cycloalkyl, aryl, or aryl(C₁₋₆ alkyl); and R₁₁ is hydrogen, C₁₋₆ alkyl, C₃₋₈ cycloalkyl, aryl, aryl(C₁₋₆ alkyl), or halo-C₁₋₆ alkyl; comprising the step iv): reacting a compound of Formula C-1, wherein X^{A} is halogen,

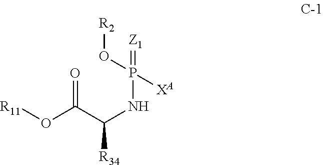

with H—W—R₁₂ in the presence of a base to generate the compound of Formula X.

In some methods, Z₁ is S.

In some methods, R₂ is optionally substituted aryl. For example, R₂ is phenyl or naphthyl optionally substituted with 1-3 of C₁₋₆ alkyl. In other examples, R₂ is unsubstituted phenyl.

In some methods, W is —S— or —O—; and R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, —W—R₁₂ is selected from

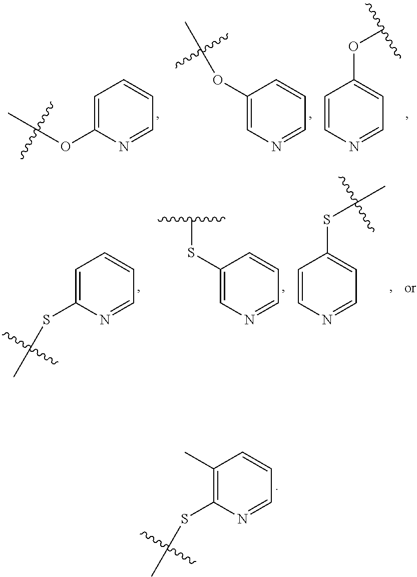

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

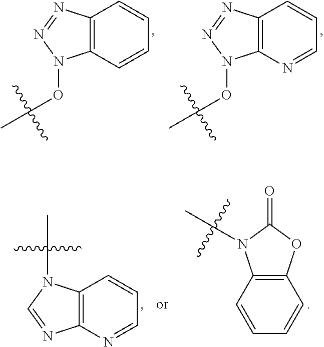 [SMILES: *On1nnc2ccccc21 ; *On1nnc2cccnc21 ; *n1cnc2ncccc21 ; *n1c(=O)oc2ccccc21]

In some methods, —W—R₁₂ is

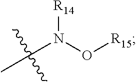

and R₁₄ and R₁₅ are each independently C₁₋₆ alkyl, cycloalkyl, or heteroalkyl, or R₁₄ and R₁₅ taken together with the heteroatoms to which they are attached form a 6-10 membered heterocyclic ring optionally substituted with 1-3 of R₁₃. For example, —W—R₁₂ is selected from

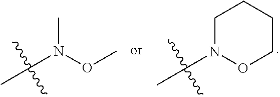

In some methods, R₁₂ is a 5-6-membered fully saturated or partially unsaturated heterocyclic ring having 1-2 heteroatoms independently selected from N, O, or S, optionally substituted with 1-3 of R₁₃. For example, —W—R₁₂ is

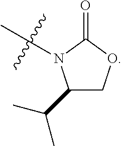 [SMILES: *N1C(=O)OC[C@H]1C(C)C]

In some methods, R₃₄ is C₁₋₆ alkyl or halo-C₁₋₆ alkyl. For example, R₃₄ is methyl, ethyl, propyl, iso-propyl, butyl, sec-butyl, or tert-butyl, any of which is optionally substituted with 1-3 halo.

In some methods, R₁₁ is hydrogen, C₁₋₆ alkyl, or C₃₋₈ cycloalkyl. For example, R₁₁ is C₁₋₆ alkyl. In other examples, R₁₁ is methyl, ethyl, n-propyl, iso-propyl, n-butyl, sec-butyl, or tert-butyl.

In some methods, the base of step iv) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iv) is performed in the presence of an aprotic organic solvent, such as any solvent or mixture of solvents described above in any of steps ia)-id).

In some methods, the reaction of step iv) is performed at a temperature of about 30° C. or less.

Some methods further comprise step v): reacting a compound of Formula BB, wherein X^{B} is halogen, with a compound of Formula C-2

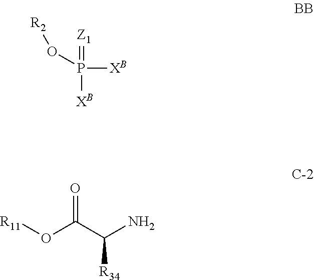

under nucleophilic substitution conditions to generate the compound of Formula C-1.

Another aspect of this application provides a compound of Formula B-1B:

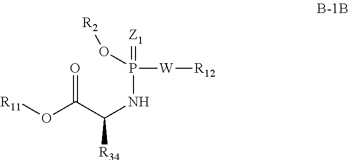

wherein Z₁ is S or O; R₂ is optionally substituted aryl or optionally substituted heteroaryl; W is a bond, —S—, or —O—; and R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl; or —W—R₁₂ is

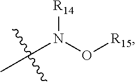

wherein each of R₁₄ and R₁₅ is independently C₁₋₆ alkyl, cycloalkyl, or heteroalkyl, or R₁₄ and R₁₅ taken together with the heteroatoms to which they are attached form a 6-10-membered heterocyclic ring optionally substituted with 1-3 of R₁₃; R₃₄ is C₁₋₆ alkyl, halo-C₁₋₆ alkyl, C₃₋₈ cycloalkyl, aryl, or aryl(C₁₋₆ alkyl); and R₁₁ is hydrogen, C₁₋₆ alkyl, C₃₋₈ cycloalkyl, aryl, aryl(C₁₋₆ alkyl), or halo-C₁₋₆ alkyl.

In some embodiments, Z₁ is S.

In other embodiments, R₂ is unsubstituted phenyl.

In some embodiments, W is —S— or —O—; and R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, —W—R₁₂ is selected from

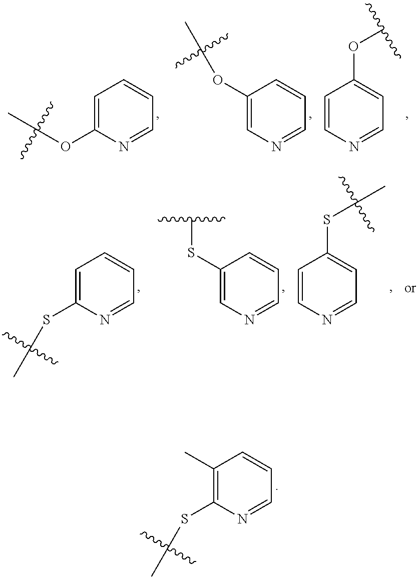

In some embodiments, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

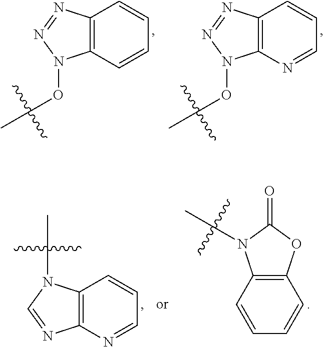 [SMILES: *On1nnc2ccccc21 ; *On1nnc2cccnc21 ; *n1cnc2ncccc21 ; *n1c(=O)oc2ccccc21]

In some embodiments, —W—R₁₂ is

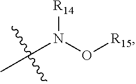

wherein each of R₁₄ and R₁₅ is independently C₁₋₆ alkyl, cycloalkyl, or heteroalkyl, or R₁₄ and R₁₅ taken together with heteroatoms to which they are attached to form a 6-10 membered heterocyclic ring. For example, —W—R₁₂ is selected from

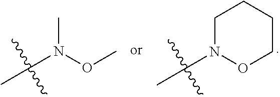

### BRIEF DESCRIPTION OF THE DRAWINGS

The following figures are provided by way of example and are not intended to limit the scope of the application.

FIG. 1A is a ¹H NMR spectrum of the compound of Formula B-4B1 from Example 2.

FIG. 1B is a ¹H NMR spectrum of the purified compound of Formula B-4B1 from Example 2.

FIG. 2A is a ³¹P NMR spectrum of the compound of Formula B-4B1 from Example 2.

FIG. 2B is a ³¹P NMR spectrum of the purified compound of Formula B-4B1 from Example 2.

FIG. 3 is a HPLC chromatogram of the compound of Formula B-4B1 from Example 2.

FIG. 4 is a ¹H NMR spectrum of the compound of Formula Va from Example 3A.

FIG. 5 is a ³¹P NMR spectrum of the compound of Formula Va from Example 3A.

FIG. 6 is a HPLC chromatogram of the compound of Formula Va from Example 3A.

FIG. 7 is a ¹H NMR spectrum of the compound of Formula V-1 from Example 3B.

FIG. 8 is a ³¹P NMR spectrum of the compound of Formula V-1 from Example 3B.

FIG. 9 is a ¹H NMR spectrum of the compound of Formula A-4A from Example 4A.

FIG. 10 is a ¹H NMR spectrum of the compound of Formula 7 from Example 4A.

### DETAILED DESCRIPTION OF THE INVENTION

The present application provides a method of preparing a compound of Formula I:

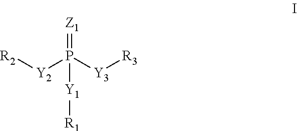

or pharmaceutically acceptable salt thereof, wherein Z₁ is O or S; each of Y₁, Y₂ and Y₃ is independently a bond, —S—, —O—, or —NR₁₀₀—, R₁₀₀ is hydrogen, C₁₋₆ alkyl, C₂₋₆ alkenyl, C₂₋₆ alkynyl, aryl, heteroaryl, aryl(C₁₋₆ alkyl), C₃₋₈ cycloaliphatic, or a saturated, partially unsaturated, or fully unsaturated 3-8 membered heterocyclic ring having up to 3 heteroatoms independently selected from N, O, or S; and each of R₁, R₂ and R₃ is independently -L-R₅, wherein each L is independently a bond, —(CH₂)ₘ—, —(CH₂)ₘ—(CHR₆)ₚ—, —(CH₂)ₘ—(CR₆R₇)ₚ—, or —(C(R₈)₂)ₘC(O)O—, each of R₆ and R₇ is independently selected from hydrogen, halogen, —OH, —N(R₈)₂, or —OR₈, each R₈ is independently hydrogen or C₁₋₆ alkyl, each m is independently 0-3, each p is independently 0-3, each R₅ is independently hydrogen, —O⁻, —OH, alkoxy, C₁₋₆ alkyl, C₂₋₆ alkenyl, C₂₋₆ alkynyl, —(C(R₈)₂)ₘC(O)OR₈, aryl, aryl(C₁₋₆ alkyl), C₃₋₈ cycloaliphatic, heteroaryl, or a saturated or partially unsaturated 3-8 membered heterocyclic ring having up to 3 heteroatoms independently selected from N, O, or S, an optionally substituted amine, an optionally substituted N-linked amino acid, an optionally substituted N-amino acid ester derivative, or

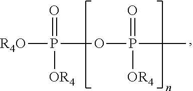

wherein each R₄ is independently absent or hydrogen, and n is 0 or 1, and wherein the alkyl, alkenyl, alkynyl, aryl, aryl-(C₁₋₆ alkyl), cycloaliphatic, heteroaryl, or heterocyclic ring groups are each optionally substituted with 1-3 groups independently selected from halo, —OH, —CN, azido, optionally substituted C₁₋₆ alkyl, optionally substituted C₁₋₆ alkoxy, an optionally substituted heterocyclic base, or an optionally substituted heterocyclic base with a protected amino group; comprising i) reacting a compound of Formula A with a compound of Formula B

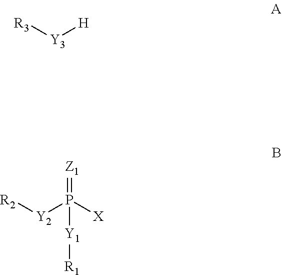

wherein X is a leaving group, in the presence of an acid or a metal salt, to generate the compound of Formula I.

### I. DEFINITIONS

As used herein, the following definitions shall apply unless otherwise indicated.

For purposes of this application, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the application may optionally be substituted with one or more substituents, such as are illustrated generally above, or as exemplified by particular classes, subclasses, and species of the application.

As used herein, the term “hydroxyl” or “hydroxy” refers to an —OH moiety.

As used herein the term “aliphatic” encompasses the terms alkyl, alkenyl, and alkynyl, each of which being optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, 1- or 2-isopropenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, a “haloaliphatic” group refers to an aliphatic group substituted with 1-3 halogen atoms on each carbon atom. For instance, the term haloalkyl includes the group —CF₃.

As used herein, an “amido” encompasses both “aminocarbonyl” and “carbonylamino”. These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, the term “azido” refers to a functional group and can be described by several resonance structures, an important one being ⁺N=N⁻=N⁺.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic”, “alkyl”, and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S-], sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, which would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophene-yl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

A “heteroaraliphatic” (such as a heteroaralkyl group) as used herein, refers to an aliphatic group (e.g., a C₁₋₄ alkyl group) that is substituted with a heteroaryl group. “Aliphatic”, “alkyl”, and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The terms “heterocycle” or “heterocyclic,” as used herein indicates a fully saturated, partially saturated, or fully unsaturated 3- to 12-membered monocyclic or bicyclic ring having from 1 to 5 ring heteroatoms selected from O, S, or N. The bicyclic heterocycles may be fused or spirocyclic ring systems. Monocyclic or bicyclic heterocycles, alone, and together with fused or spirocyclic groups, include aziridines, oxirane, azetidine, azirine, thirene, oxetane, oxazetidine, tetrazole, oxadiazole, thiadiazole, triazole, isoxazole, oxazole, oxathiazole, oxadiazolone, isothiazole, thiazole, imidazole, pyrazole, isopyrazole, diazine, oxazine, dioxazine, oxadiazine, thiadiazine, oxathiazole, triazine, thiazine, dithiazine, tetrazine, pentazine, pyrazolidine, pyrrole, pyrrolidine, furan, thiophene, isothiophene, tetrazine, triazine, morpholine, thiazine, piperazine, pyrazine, pyridazine, pyrimidine, piperidine, pyridine, pyran, thiopyran, azepine, diazepine, triazepine, azepane, 3-aza-bicylco[3.2.1]octane, 2-lo aza-bicylco[2.2.1]heptane, octahydrocyclopentapyrrole, aza-bicyclo-nonane, indole, indoline, isoindoline, indolizine, octahydro-isoindole, 2-azaspiro[4.5]decane, 6-azaspiro[2.5]octane, 7-azaspiro[3.5]nonane, 8-azaspiro[4.5]decane, 3-asaspiro[5.5]undecane, 1-oxa-7-azaspiro[4.4]nonane, 1-oxa-8-azaspiro[4.5]decane, purine, benzothiazole, benzoxazole, indazole, benzofuran, and isobenzofuran. Examples of spirocyclic heterocycles include oxaspiro[2.3]hexaneI 1-oxaspiro[3.4]octane, 1-oxaspiro[2.5]octaneI 2-oxaspiro[4.5]decane, 2,6-diazaspiro[3.2]heptane, azaspiro[2.5]octane, 6-aza-spiro[2.5]octane, 1,6-diazaspiro[2.5]octane, 7-aza-spiro[3.5]nonane, 3-aza-spiro[5.5]undecane, 8-azaspiro[4.5]decane, 1,3-diazaspiro[4.5]decane, 2,8-diazaspiro[5.5]hendecaneI 3,9-diazaspir0[5.5]hendecane, and 1-ox-6-azaspiro[2.5]octane. It will be understood that the terms listed above for heterocycles includes each possible atomic orientation for the groups listed. For instance, the term oxadiazole includes 1,2,3-oxadiazole, 1,3,4-oxadiazole and 1,2,4-oxadiazole; the term thiadiazole includes 1,2,3-thiadiazole, 1,3,4-thiadiazole and 1,2,4-thiadiazole. The term “heterocyclyl” refers to a heterocycle radical.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z}, wherein R^{X} and e have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—S(O)—R^{X} or —S(O)—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein RP is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ- where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

As used herein, “Dess-Martin periodinane” and its abbreviation “DMP” are used interchangeably. DMP refers to 1,1,1-triacetoxy-1,1-dihydro-1,2-benziodoxol-3(1H)-one having the structure

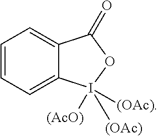

The phrase “optionally substituted” is used interchangeably with the phrase “substituted or unsubstituted.” As described herein, compounds of the application can optionally be substituted with one or more substituents, such as are illustrated generally above, or as exemplified by particular classes, subclasses, and species of the application. As described herein, the variables R₁-R₃₄ and other variables contained in Formulae I, II, II-1, III, IV, V, X, and X-1 described herein encompass specific groups, such as alkyl and aryl. Unless otherwise noted, each of the specific groups for the variables R₁-R₃₄ and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this application are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, “chemical purity” refers to the degree to which a substance, i.e., the desired product or intermediate, is undiluted or unmixed with extraneous material such as chemical byproducts.

As used herein, “d.r.” refers to diastereomeric ratio.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Unless otherwise stated, all tautomeric forms of compounds of the application are within the scope of the application. For example all tautomers of a phosphate and a phosphorothioate groups are intended to be included. Examples of tautomers of a phosphorothioate groups are intended to be included. Examples of tautomers of a phosphorothioate include the following:

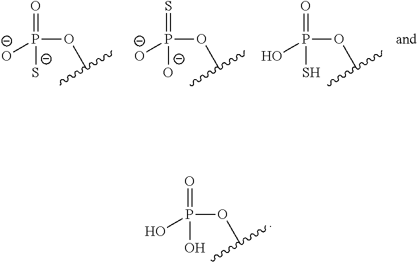 [SMILES: *OP(=O)([O-])[S-] ; *OP([O-])([O-])=S ; *OP(=O)(O)S ; *OP(=O)(O)O]

Furthermore, all tautomers of heterocyclic bases known in the art are intended to be included, including of natural and unnatural purine bases and pyrimidine bases.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this application. Such compounds are useful, for example, as analytical tools or probes in biological assays, or as therapeutic agents.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., tert-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilypethoxylmethyl or t-butyldiphenylsilyl); esters (e.g., benzoate ester); carbonates (e.g., methoxymethylcarbonate); sulfonates (e.g., tosylate or mesylate); acyclic ketal (e.g., dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁₋₇ alkylamine, cyclohexylamine, triethanolamine, ethylenediamine, and salts with amino acids such as arginine and lysine.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### II. COMMONLY USED ABBREVIATIONS

ACN acetonitrile

tBuOAc tert-butyl acetate

DABCO 1,4-diazabicyclo[2.2.2]octane

DCM dichloromethane

EtOAc ethyl acetate

IPAc iso-propyl acetate

MIBK methyl iso-butyl ketone

TEA triethylamine

THF tetrahydrofuran

PG protecting group

LG leaving group

Ac acetyl

TMS trimethylsilyl

TBS tert-butyldimethylsilyl

TIPS tri-iso-propylsilyl

TBDPS tert-butyldiphenylsilyl

TOM tri-iso-propylsilyloxymethyl

DMP Dess-Martin periodinane

IBX 2-iodoxybenzoic acid

DMF dimethylformamide

MTBE methyl-tert-butylether

TBAF tetra-n-butylammonium fluoride

d.e. diastereomeric excess

e.e. enantiomeric excess

d.r. diastereomeric ratio

DMSO dimethyl sulfoxide

TCA trichloroacetic acid

ATP adenosine triphosphate

EtOH ethanol

Ph phenyl

Me methyl

Et ethyl

Bu butyl

DEAD diethylazodicarboxylate

HEPES 4-(2-hydroxyethyl)-1-piperazineethanesulfonic acid

DTT dithiothreitol

MOPS 4-morpholinepropanesulfonic acid

NMR nuclear magnetic resonance

HPLC high performance liquid chromatography

LCMS liquid chromatography-mass spectrometry

TLC thin layer chromatography

Rt retention time

HOBt hydroxybenzotriazole

Ms mesyl

Ts tosyl

Tf triflyl

Bs besyl

Ns nosyl

Cbz carboxybenzyl

Moz p-methoxybenzyl carbonyl

Boc tert-butyloxycarbonyl

Fmoc 9-fluorenylmethyloxycarbonyl

Bz benzoyl

Bn benzyl

PMB p-methoxybenzyl

AUC area under the curve

DMPM 3,4-dimethoxybenzyl

PMP p-methoxyphenyl

### III. METHODS

It is noted that the steps recited herein may be performed in any chronological order without regard to step numbering. For example, step iii) may precede or follow step i).

In one aspect, the present application provides a method of preparing a compound of Formula I:

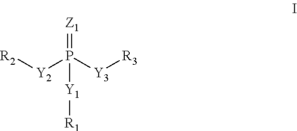

or pharmaceutically acceptable salt thereof, wherein Z₁ is O or S; each of Y₁, Y₂ and Y₃ is independently a bond, —S—, —O—, or —NR₁₀₀—; R₁₀₀ is hydrogen, C₁₋₆ alkyl, C₂₋₆ alkenyl, C₂₋₆ alkynyl, aryl, heteroaryl, aryl(C₁₋₆ alkyl), C₃₋₈ cycloaliphatic, or a saturated, partially unsaturated, or fully unsaturated 3-8 membered heterocyclic ring having up to 3 heteroatoms independently selected from N, O, or S; each of R₁, R₂ and R₃ is independently -L-R₅; wherein L is a bond, —(CH₂)ₘ—, —(CH₂)ₘ—(CHR₆)ₚ—, or —(CH₂)ₘ,—(CR₆R₇)ₚ—, —(C(R₈)₂)ₘC(O)O—; wherein R₆ and R₇ are each independently selected from hydrogen, halogen, —OH, —N(R₈)₂, or —OR₈, R₈ is hydrogen or C₁₋₆ alkyl, each m is independently 0-3, and each p is independently 0-3 and R₅ is hydrogen, —O⁻, —OH, alkoxy, C₁₋₆ alkyl, C₂₋₆ alkenyl, C₂₋₆ alkynyl, —(C(R₈)₂)ₘC(O)OR₈, aryl, heteroaryl, aryl(C₁₋₆ alkyl), C₃₋₈ cycloaliphatic, or a saturated, partially unsaturated, or fully unsaturated 3-8 membered heterocyclic ring having up to 3 heteroatoms independently selected from N, O, or S, wherein the alkyl, alkenyl, alkynyl, aryl, aryl-(C₁₋₆ alkyl), cycloaliphatic, or heterocyclic ring groups are each optionally substituted with 1-3 groups independently selected from halo, —OH, —CN, azido, optionally substituted C₁₋₆ alkyl, optionally substituted C₁₋₆ alkoxy, an optionally substituted heterocyclic base, or an optionally substituted heterocyclic base with a protected amino group, an optionally substituted amine, an optionally substituted N-linked amino acid, an optionally substituted N-amino acid ester derivative, or

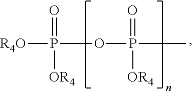

wherein each R₄ is independently absent or hydrogen, and n is 0 or 1, comprising step i): reacting a compound of Formula A with a compound of Formula B

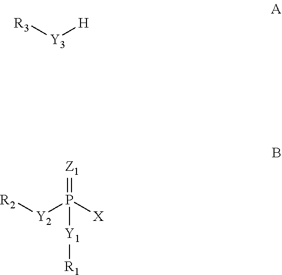

wherein X is a leaving group, in the presence of an acid or a metal salt, to generate the compound of Formula I.

In some methods, Y₁ is a bond; Y₂ and Y₃ are each independently —O—, or —S—; R₁ is O⁻, —OH, alkoxy, an optionally substituted amine, an optionally substituted N-linked amino acid or an optionally substituted N-amino acid ester derivative; and R₂ and R₃ are each independently hydrogen, C₁₋₆ alkyl, aryl, heteroaryl, aryl(C₁₋₆ alkyl), or C₃₋₈ cycloaliphatic.

In some methods, —Y₁—R₁ is an optionally substituted N-linked amino acid or an optionally substituted N-amino acid ester derivative. For example, —Y₁—R₁ is

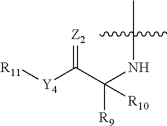

wherein Z₂ is O or S; Y₄ is a bond, —S—, —O—, or —NR₅—; each of R₉ and R₁₀ is independently selected from hydrogen, C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl, or R₉ and R₁₀ taken together with the carbon atom to which they are attached form a C₃₋₆ cycloalkyl; and R₁₁ is hydrogen, C₁₋₆ alkyl, C₃₋₈ cycloalkyl, aryl, aryl(C₁₋₆ alkyl), or halo-C₁₋₆ alkyl.

In some methods, one of R₉ and R₁₀ is hydrogen and the other is selected from C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl. For example, R₉ is hydrogen and R₁₀ is selected from C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl. In other examples, R₉ is hydrogen and R₁₀ is C₁₋₆ alkyl or halo-C₁₋₆ alkyl. And, in some examples, R₉ is hydrogen and R₁₀ is selected from methyl, ethyl, n-propyl, iso-propyl, n-butyl, sec-butyl, tert-butyl, or neohexyl.

In some methods, R₁₁ is C₁₋₆ alkyl, C₃₋₈ cycloalkyl, aryl, aryl(C₁₋₆ alkyl), or halo-C₁₋₆ alkyl. For example, R₁₁ is C₁₋₆ alkyl or C₃₋₈ cycloalkyl. In other examples, R₁₁ is methyl, ethyl, n-propyl, iso-propyl, n-butyl, sec-butyl, tert-butyl, or neohexyl.

In other methods, —Y₁—R₁ is

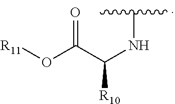

In some methods, R₂ is optionally substituted aryl. For example, R₂ is optionally substituted phenyl or optionally substituted naphthyl. In other examples, R₂ is phenyl or naphthyl, either of which are optionally substituted with 1-3 of C₁₋₆ alkyl. In other embodiments, R₂ is unsubstituted phenyl.

In some methods, the reaction of step i) occurs in the presence of an acid. For example, the reaction of step i) occurs in the presence of an acid, and the acid is a strong organic acid. In some examples, the reaction of step i) occurs in the presence of trifluoromethanesulfonic acid or methanesulfonic acid.

In some methods, the reaction of step i) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step i) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step i) is performed in the presence of a metal salt of fluoroborate. In some examples, the metal salt of fluoroborate is silver tetrafluoroborate, silver hexafluorophosphate, or any combination thereof.

In some methods, wherein the group X is —W—R₁₂, the compound of Formula B is a compound of Formula B-1:

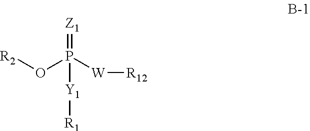

wherein W is a bond, —S—, or —O—; and R₁₂ is a 5-10-membered mono- or bicyclic saturated, partially unsaturated, a fully unsaturated heterocyclic ring having 1-3 heteroatoms independently selected from N, O, or S, optionally substituted with 1-3 of R₁₃, wherein R₁₃ is oxo or an optionally substituted C₁₋₆ alkyl.

In some methods, R₁₂ is a monocyclic saturated heterocyclic ring having 1-3 heteroatoms independently selected from N, O, or S, optionally substituted with 1-3 of R₁₃, wherein R₁₃ is an optionally substituted C₁₋₆ alkyl. For example, R₁₂ is oxazolidin-2-one, either of which is optionally substituted with C₁₋₄ alkyl. In other examples, —W—R₁₂ is

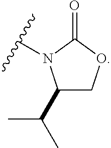 [SMILES: *N1C(=O)OC[C@H]1C(C)C]

In some methods, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

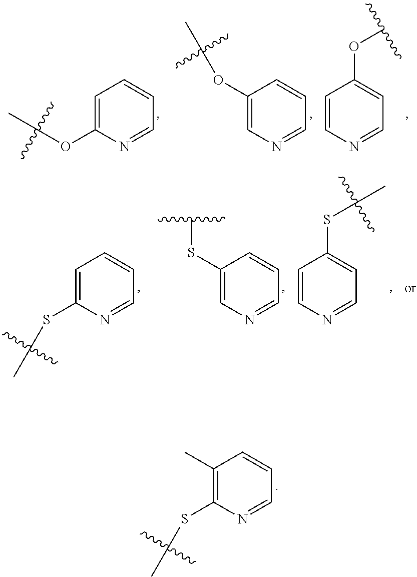

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, —W—R₁₂ is selected from

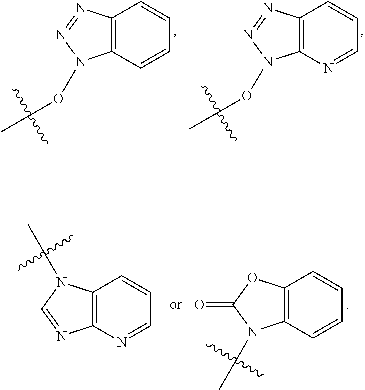 [SMILES: *On1nnc2ccccc21 ; *On1nnc2cccnc21 ; *n1cnc2ncccc21 ; *n1c(=O)oc2ccccc21]

In some methods, —W—R₁₂ is

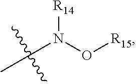

wherein R₁₄ and R₁₅ are each independently C₁₋₆ alkyl, cycloalkyl, or heteroalkyl, or R₁₄ and R₁₅ taken together with N and O to form a 6-10 membered heterocyclic ring optionally substituted with 1-3 of R₁₃. For example, —W—R₁₂ is selected from

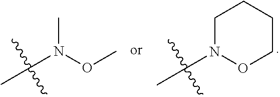

In some methods, the compound of Formula B-1 is a compound of Formula B-2a or B-2b:

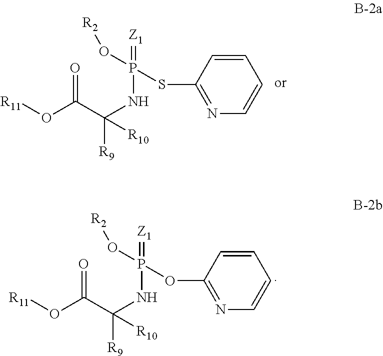

In some methods, the reaction of step i) occurs in the presence of an organic solvent. In some examples, the organic solvent of step i) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step i) is performed at a temperature of about 30° C. or less. For example, the reaction of step i) is performed at a temperature of from about −20° C. to about 25° C.

Some methods further comprise step ii): reacting a compound of Formula B-3, wherein X^{A} is halogen, with H—W—R₁₂

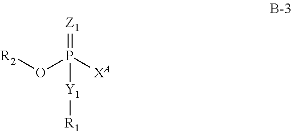

in the presence of a base to generate the compound of Formula B-1.

In some methods, the base of step ii) is am amine base. For example, the base of step ii) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step ii) is performed in the presence of an organic solvent. For example, the organic solvent of step ii) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step ii) is performed at a temperature of about 30° C. or less. For example, the reaction of step ii) is performed at a temperature of from about −10° C. to about 25° C.

In some methods, the compound of Formula B-3 is a compound of Formula B-4, wherein X^{A} is halogen:

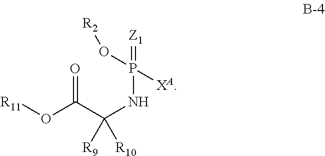

Some methods further comprise step iii): reacting a compound of Formula B-5, wherein X^{B} is halogen, with a compound of Formula C:

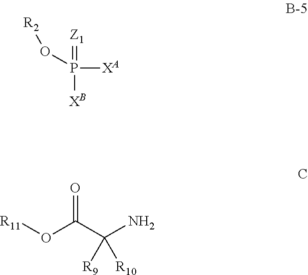

under nucleophilic substitution conditions to generate the compound of Formula B-4.

Another aspect of this application provides a method of preparing a compound of Formula II:

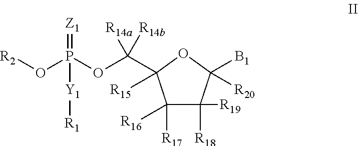

or a pharmaceutically acceptable salt thereof; wherein Z₁ is S or O; B₁ is an optionally substituted heterocyclic base or an optionally substituted heterocyclic base with a protected amino group; Y₁—R₁ is —O⁻, —OH, alkoxy, an optionally substituted amine, an optionally substituted N-linked amino acid or an optionally substituted N-amino acid ester derivative; R₂ is an optionally substituted aryl, an optionally substituted heteroaryl, an optionally substituted heterocyclyl or

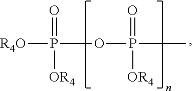

wherein each R₄ is independently absent or hydrogen, and n is 0 or 1; each of R₁₄ₐ and R_{14b} is independently selected from hydrogen, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or R₁₄ₐ and R_{14b} taken together with the carbon atom to which they are attached form an optionally substituted C₃₋₆ cycloalkyl; R₁₅ is hydrogen, azido, an optionally substituted C₁₋₆ alkyl, an optionally substituted C₂₋₆ alkenyl, or an optionally substituted C₂₋₆ alkynyl; each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or R₁₇ and R₁₈ are both oxygen atoms that are linked together by a carbonyl group; R₂₀ is hydrogen, halogen, azido, cyano, an optionally substituted C₁₋₆ alkyl, or —OR₂₀; each of R₂₁ and R₂₂ is independently selected from hydrogen, optionally substituted C₁₋₆ alkyl or optionally substituted C₃₋₆ cycloalkyl; comprising the step i): reacting a compound of Formula A-1 with a compound of Formula B-X

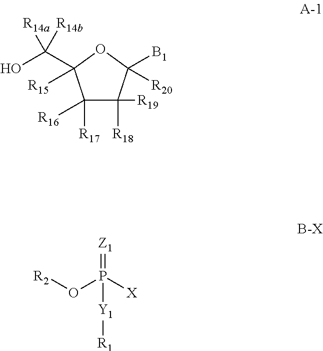

wherein X is a leaving group capable of being displaced by a —OH group, in the presence of an acid or salt, to generate the compound of Formula II.

In some methods, B₁ is an optionally substituted saturated or partially unsaturated 5-7-membered monocyclic heterocycle having at least 1 nitrogen atom and 0 to 2 additional heteroatoms independently selected from N, O, or S; or B₁ is an optionally substituted saturated or partially unsaturated 8-10-membered bicyclic heterocycle having at least 2 nitrogen atoms and 0 to 3 additional heteroatoms independently selected from N, O, or S. For example, B₁ is selected from

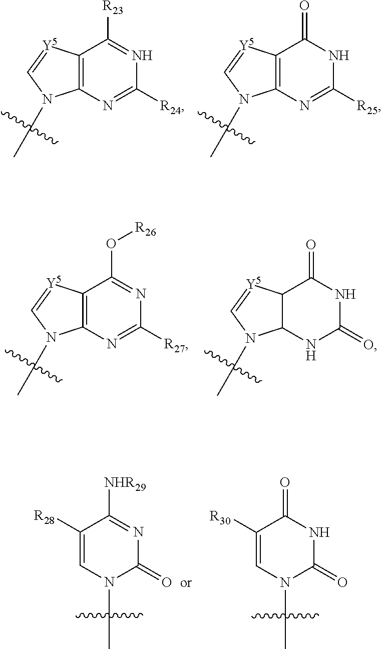

wherein Y⁵ is ═N— or ═CR₃₁—, wherein R₃₁ is C₁₋₆ alkyl, or C₂₋₆ alkenyl; R₂₃ is halogen or —NHR₃₂, wherein R₃₂ is hydrogen, C₁₋₆ alkyl, C₂₋₆ alkenyl, C₃₋₈ cycloalkyl, —O—C₁₋₆ alkyl, —C(O)R^{A}, or —C(O)OR^{A}; R₂₄ is hydrogen, halogen, or —NHR₃₃; R₂₅ is hydrogen or —NHR₃₃; R₂₆ is hydrogen, halogen, C₁₋₆ alkyl, or C₂₋₆ alkenyl; R₂₇ is hydrogen, C₁₋₆ alkyl, C₃₋₈ cycloalkyl, —C(O)R^{A}, or —C(O)OR^{A}; R₂₈ is hydrogen, halogen, C₁₋₆ alkyl, or C₂₋₆ alkenyl; R₂₉ is hydrogen, halogen, C₁₋₆ alkyl, or C₂₋₆ alkenyl; R₃₀ is hydrogen, halogen, —NHR₃₃, C₁₋₆ alkyl, or C₂₋₆ alkenyl; each R₃₃ is independently selected from hydrogen, —C(O)R^{A}, or —C(O)OR^{A}; and each R^{A} is independently selected from C₁₋₆ alkyl, C₂₋₆ alkenyl, C₃₋₈ cycloalkyl, aryl, heteroaryl, heterocyclyl, aryl(C₁₋₆ alkyl), heteroaryl(C₁₋₆ alkyl), or heterocyclyl(C₁₋₆ alkyl). In other examples, B₁ is selected from

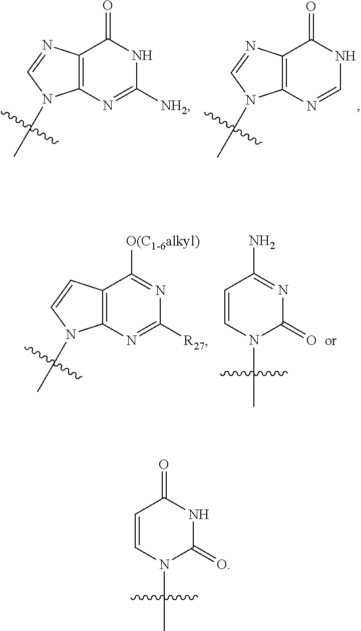

In some methods, —Y₁—R₁ is

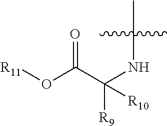

wherein each of R₉ and R₁₀ is independently selected from hydrogen, C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl, or R₉ and R₁₀ taken together with the carbon atom to which they are attached form a C₃₋₆ cycloalkyl; and R₁₁ is hydrogen, C₁₋₆ alkyl, C₃₋₈ cycloalkyl, aryl, aryl(C₁₋₆ alkyl), or halo-C₁₋₆ alkyl.

In some methods, R₂ is optionally substituted aryl or optionally substituted heteroaryl. For example, R₂ is optionally substituted aryl. In other examples, R₂ is unsubstituted phenyl.

In some methods, the reaction of step ia) occurs in the presence of an acid. In some examples, the acid is a strong organic acid. In other examples, acid is trifluoromethanesulfonic acid or methanesulfonic acid.

In some methods, the reaction of step ia) occurs in the presence of a metal salt. In some examples, the salt is a metal salt of trifluoromethanesulfonate, a metal salt of acetate, or a metal salt of fluoroborate. In other examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step ia) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step ia) is performed in the presence of a metal salt of fluoroborate. In some examples, the metal salt of fluoroborate is silver tetrafluoroborate, silver hexafluorophosphate, or any combination thereof.

In some methods, the compound of Formula B is a compound of Formula B-1:

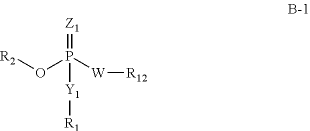

wherein W is a bond, —S—, or —O—; and R₁₂ is a 5-10-membered mono- or bicyclic saturated, partially unsaturated, or fully unsaturated heterocyclic ring having 1-3 heteroatoms independently selected from N, O, or S, optionally substituted with 1-3 of R₁₃, wherein R₁₃ is oxo or an optionally substituted C₁₋₆ alkyl.

In some methods, R₁₂ is a monocyclic saturated heterocycle having 1-3 heteroatoms independently selected from N, O, or S, optionally substituted with 1-3 of R₁₃, wherein R₁₃ is oxo or an optionally substituted C₁₋₆ alkyl. For example, R₁₂ is oxazolidin-2-one, either of which is optionally substituted with C₁₋₄ alkyl.

In some methods, —W—R₁₂ is

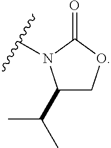 [SMILES: *N1C(=O)OC[C@H]1C(C)C]

In some methods, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

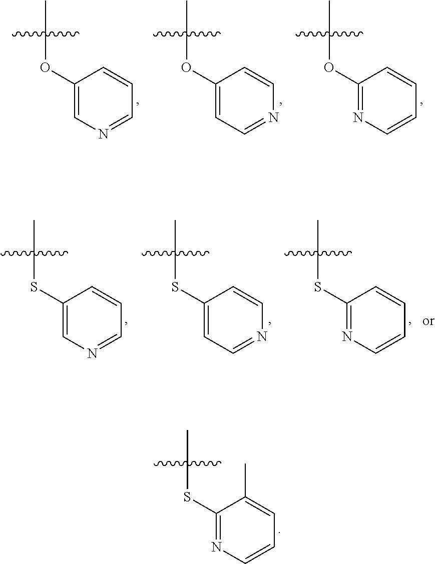

In some methods, R₁₂ is an 8-10-membered bicycliccyclic heteroaryl having 1-4 heteroatoms independently selected from N, O, or S, optionally substituted with 1-3 of R₁₃, wherein R₁₃ is an optionally substituted C₁₋₆ alkyl.

In some methods, —W—R₁₂ is selected from

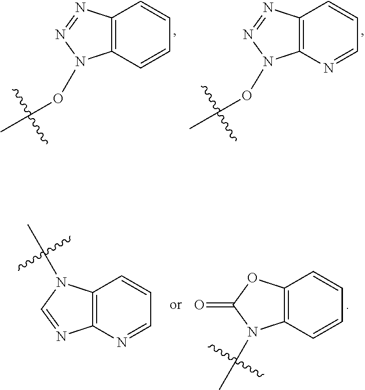 [SMILES: *On1nnc2ccccc21 ; *On1nnc2cccnc21 ; *n1cnc2ncccc21 ; *n1c(=O)oc2ccccc21]

In some methods, —W—R₁₂ is

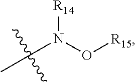

wherein R₁₄ and R₁₅ are each independently C₁₋₆ alkyl, cycloalkyl, or heteroalkyl, or R₁₄ and R₁₅, taken together with the heteroatoms to which they are attached form, a 6-10 membered heterocyclic ring optionally substituted with 1-3 of R₁₃.

In some methods, —W—R₁₂ is

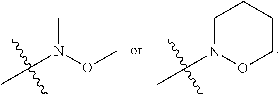

In some methods, the compound of Formula B-1 is a compound of Formula B-2a or B-2b:

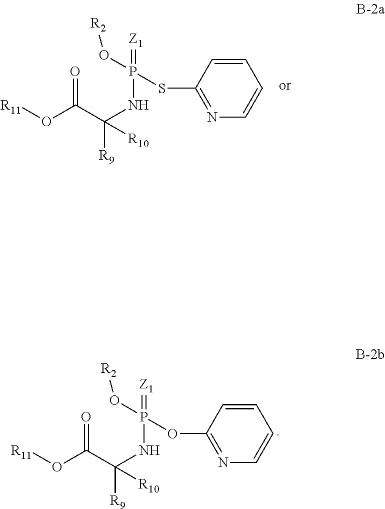

In some methods, the reaction of step ia) occurs in the presence of an organic solvent. In some examples, the organic solvent of step ia) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ia) is performed at a temperature of about 30° C. or less. For example, the reaction of step ia) is performed at a temperature of from about −20° C. to about 25° C.

Some methods further comprise step ii): reacting a compound of Formula B-3, wherein X^{A} is halogen, with H—W—R₁₂

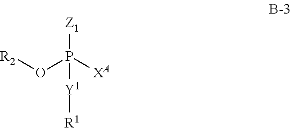

in the presence of a base to generate the compound of Formula B-1.

In some methods, the base of step ii) is an amine base. For example, the base of step ii) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step ii) is performed in the presence of an organic solvent. For example, the organic solvent of step ii) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step ii) is performed at a temperature of about 30° C. or less. For example, the reaction of step ii) is performed at a temperature of from about −10° C. to about 25° C.

In some methods, the compound of Formula B-3 is a compound of Formula B-4, wherein X^{A} is halogen:

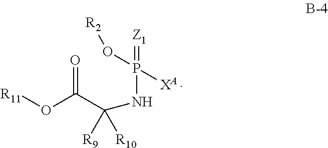

Some methods further comprise step iii): reacting a compound of Formula B-5, wherein X^{B} is halogen, with a compound of Formula C:

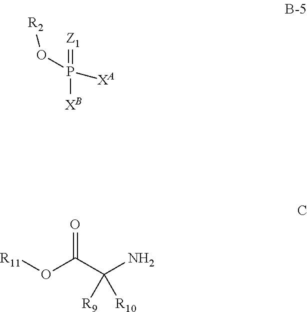

under nucleophilic substitution conditions to generate the compound of Formula B-4.

Another aspect of this application provides a method of preparing a compound of Formula III:

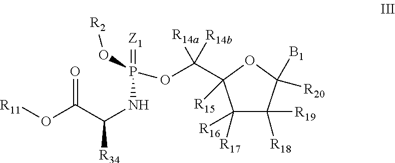

or a pharmaceutically acceptable salt thereof, having a diastereomeric purity of about 70% or greater (e.g., about 75% or greater or about 80% or greater), wherein Z₁ is S or O; B₁ is an optionally substituted heterocyclic base or an optionally substituted heterocyclic base with a protected amino group; R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; R₂ is an optionally substituted aryl, an optionally substituted heteroaryl, an optionally substituted heterocyclyl or

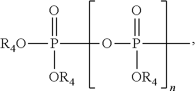

wherein each R₄ is independently absent or hydrogen, and n is 0 or 1; each of R₁₄ₐ and R_{14b} is independently selected from hydrogen, deuterium, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or R₁₄ₐ and R_{14b} taken together with the carbon atom to which they are attached form an optionally substituted C₃₋₆ cycloalkyl; R₁₅ is hydrogen, azido, an optionally substituted C₁₋₆ alkyl, an optionally substituted C₂₋₆ alkenyl, or an optionally substituted C₂₋₆ alkynyl; each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or R₁₇ and R₁₈ are both oxygen atoms that are linked together by a carbonyl group; R₂₀ is hydrogen, halogen, azido, cyano, an optionally substituted C₁₋₆ alkyl, or —OR₂₁; each of R₂₁ and R₂₂ is independently selected from hydrogen, optionally substituted C₁₋₆ alkyl or optionally substituted C₃₋₆ cycloalkyl; comprising step ib): reacting a compound of Formula A-1 and a compound of Formula B-1B

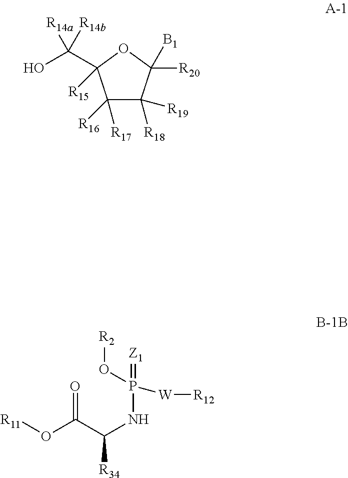

in the presence of an acid or a salt, wherein W is —S— or —O—; and R₁₂ is a 6-10-membered mono- or bicyclic saturated, partially unsaturated, or fully unsaturated heterocyclic ring having 1-4 heteroatoms independently selected from N, O, or S, wherein R₁₂ is optionally substituted with 1-3 of R₁₃ (e.g., 1-2 of C₁₋₆ alkyl) to generate the compound of Formula III. In some methods, B₁ is an optionally substituted saturated or partially unsaturated 5-7-membered monocyclic heterocycle having at least 1 nitrogen atom and 0 to 2 additional heteroatoms independently selected from N, O, or S; or B₁ is an optionally substituted saturated or partially unsaturated 8-10-membered bicyclic heterocycle having at least 2 nitrogen atoms and 0 to 3 additional heteroatoms independently selected from N, O, or S. For example, B₁ is selected from

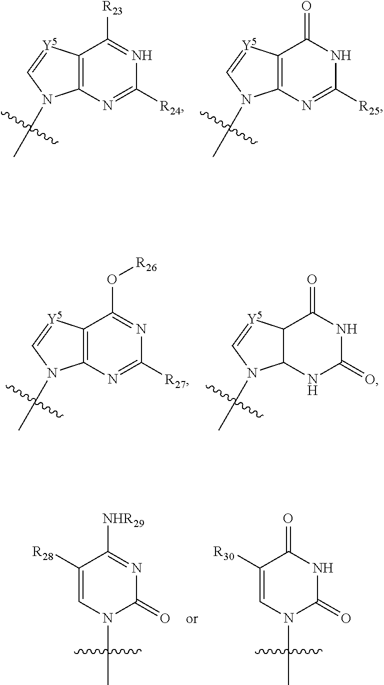

wherein Y⁵ is ═N— or ═CR₃₁—, wherein R₃₁ is C₁₋₆ alkyl, or C₂₋₆ alkenyl; R₂₃ is halogen or —NHR₃₂, wherein R₃₂ is hydrogen, C₁₋₆ alkyl, C₂₋₆ alkenyl, C₃₋₈ cycloalkyl, —O—C₁₋₆ alkyl, —C(O)R^{A}, or —C(O)OR^{A}; R₂₄ is hydrogen, halogen, or —NHR₃₃; R₂₅ is hydrogen or —NHR₃₃; R₂₆ is hydrogen, halogen, C₁₋₆ alkyl, or C₂₋₆ alkenyl; R₂₇ is hydrogen, C₁₋₆ alkyl, C₃₋₈ cycloalkyl, —C(O)R^{A}, or —C(O)OR^{A}; R₂₈ is hydrogen, halogen, C₁₋₆ alkyl, or C₂₋₆ alkenyl; R₂₉ is hydrogen, halogen, C₁₋₆ alkyl, or C₂₋₆ alkenyl; R₃₀ is hydrogen, halogen, —NHR₃₃, C₁₋₆ alkyl, or C₂₋₆ alkenyl; each R₃₃ is independently selected from hydrogen, —C(O)R^{A}, or —C(O)OR^{A}; and each R^{A} is independently selected from C₁₋₆ alkyl, C₂₋₆ alkenyl, C₃₋₈ cycloalkyl, aryl, heteroaryl, heterocyclyl, aryl(C₁₋₆ alkyl), heteroaryl(C₁₋₆ alkyl), or heterocyclyl(C₁₋₆ alkyl). In other examples, B₁ is selected from

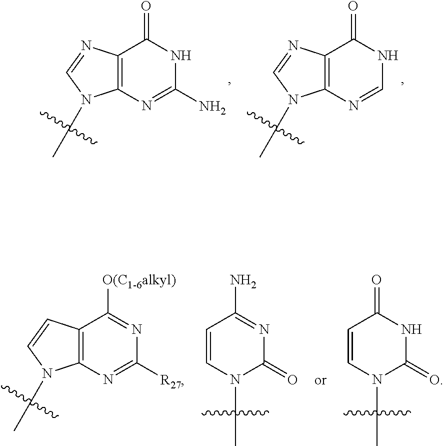

In some methods, R₂ is optionally substituted aryl. For example, R₂ is naphthyl or phenyl either of which is optionally substituted with 1-3 C₁₋₆ alkyl groups. In other examples, R₂ is unsubstituted phenyl.

In some methods, W is a bond, —S—, or —O—. For example, W is —S— or —O—.

In some methods, R₁₂ is a monocyclic saturated heterocyclic ring having 1-3 heteroatoms independently selected from N, O, or S, optionally substituted with 1-3 of R₁₃, wherein R₁₃ is oxo or an optionally substituted C₁₋₆ alkyl. For example, R₁₂ is oxazolidin-2-one optionally substituted with C₁₋₄ alkyl.

In some methods, —W—R₁₂ is

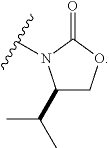 [SMILES: *N1C(=O)OC[C@H]1C(C)C]

In some methods, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

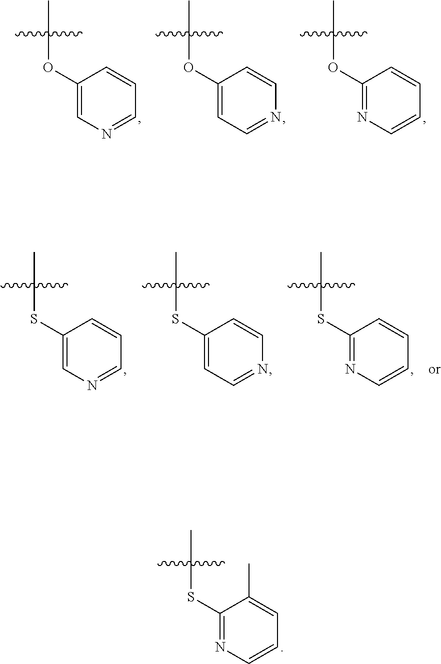

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

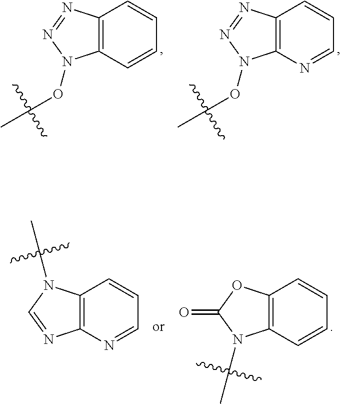 [SMILES: *On1nnc2ccccc21 ; *On1nnc2cccnc21 ; *n1cnc2ncccc21 ; *n1c(=O)oc2ccccc21]

In some methods, —W—R₁₂ is

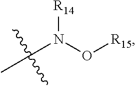

wherein R₁₄ and R₁₅ are each independently C₁₋₆ alkyl, cycloalkyl, or heteroalkyl, or R₁₄ and R₁₅, taken together with the heteroatoms to which they are attached, form a 6-10 membered heterocyclic ring optionally substituted with 1-3 of R₁₃.

In some methods, —W—R₁₂ is selected from

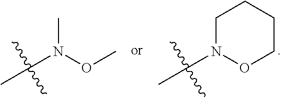

In some methods, the reaction of step ib) is performed in the presence of an acid, and the acid is a strong organic acid. For example, the acid is trifluoromethanesulfonic acid or methanesulfonic acid.

In some methods, the reaction of step ib) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step ib) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step ib) is performed in the presence of a metal salt of fluoroborate. In some examples, the metal salt of fluoroborate is silver tetrafluoroborate, silver hexafluorophosphate, or any combination thereof.

In some methods, the reaction of step ib) occurs in the presence of an organic solvent. For example, the organic solvent of step ib) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ib) is performed at a temperature of about 30° C. or less. For example, the reaction of step ib) is performed at a temperature of from about −20° C. to about 25° C.

Some methods further comprise step iib): reacting a compound of Formula C-1, wherein X^{A} is halogen, with H—W—R₁₂

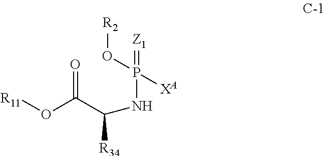

in the presence of a base to generate the compound of Formula B-1B.

In some methods, the base of step iib) is an amine base. For example, the base of step iib) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iib) is performed in the presence of an organic solvent. For example, the organic solvent of step iib) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, Cert-butyl acetate, iso-propyl acetate, methyl-iso-butyl ketone, 2-methyltetrahydrofuran, heptane, or any combination thereof.

In some methods, the reaction of step iib) is performed at a temperature of about 30° C. or less. For example, the reaction of step iib) is performed at a temperature of from about −10° C. to about 25° C.

Some methods further comprise iiib): reacting a compound of Formula B-5, wherein X^{B} is halogen, with a compound of Formula C-2

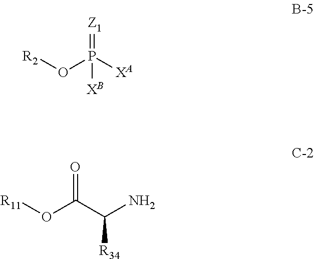

under nucleophilic substitution conditions to generate the compound of Formula C-1.

Another aspect of this application provides a method of preparing a compound of Formula Ma:

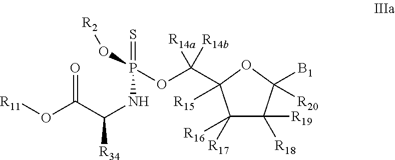

or a pharmaceutically acceptable salt thereof, having a diastereomeric purity of about 70% or greater (e.g., about 75% or greater or about 80% or greater), wherein B₁ is an optionally substituted heterocyclic base or an optionally substituted heterocyclic base with a protected amino group; R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; R₂ is an optionally substituted aryl, an optionally substituted heteroaryl, an optionally substituted heterocyclyl or

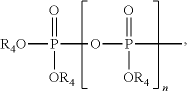

wherein each R₄ is independently absent or hydrogen, and n is 0 or 1; each of R₁₄ₐ and R_{14b} is independently selected from hydrogen, deuterium, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or R₁₄ₐ and R_{14b} taken together with the carbon atom to which they are attached form an optionally substituted C₃₋₆ cycloalkyl; R₁₅ is hydrogen, azido, an optionally substituted C₁₋₆ alkyl, an optionally substituted C₂₋₆ alkenyl, or an optionally substituted C₂₋₆ alkynyl; each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or R₁₇ and R₁₈ are both oxygen atoms that are linked together by a carbonyl group; R₂₀ is hydrogen, halogen, azido, cyano, an optionally substituted C₁₋₆ alkyl, or —OR₂₁; each of R₂₁ and R₂₂ is independently selected from hydrogen, optionally substituted C₁₋₆ alkyl or optionally substituted C₃₋₆ cycloalkyl; comprising step ib): reacting a compound of Formula A-1 and a compound of Formula B-1Ba

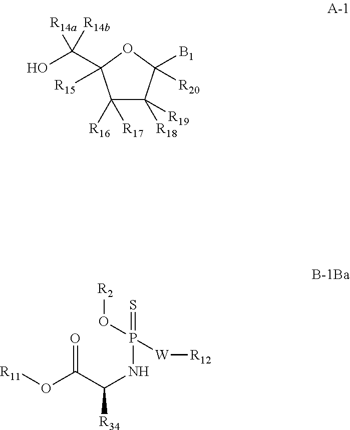

in the presence of an acid or a salt, wherein W is —S— or —O—; and R₁₂ is a 6-10-membered mono- or bicyclic saturated, partially unsaturated, or fully unsaturated heterocyclic ring having 1-4 heteroatoms independently selected from N, O, or S, wherein R₁₂ is optionally substituted with 1-3 of R₁₃ (e.g., 1-2 of C₁₋₆ alkyl) to generate the compound of Formula III. In some methods, B₁ is an optionally substituted saturated or partially unsaturated 5-7-membered monocyclic heterocycle having at least 1 nitrogen atom and 0 to 2 additional heteroatoms independently selected from N, O, or S; or B₁ is an optionally substituted saturated or partially unsaturated 8-10-membered bicyclic heterocycle having at least 2 nitrogen atoms and 0 to 3 additional heteroatoms independently selected from N, O, or S. For example, B₁ is selected from

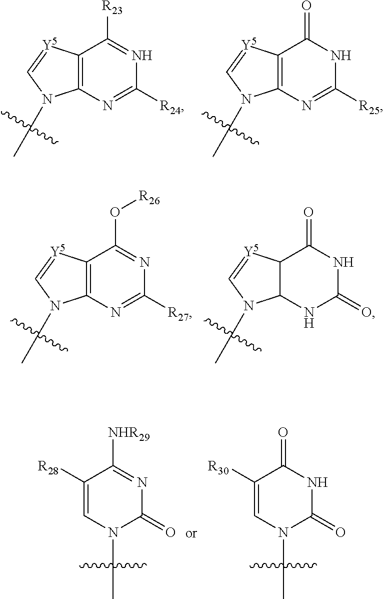

wherein Y⁵ is ═N— or ═CR₃₁—, wherein R₃₁ is C₁₋₆ alkyl, or C₂₋₆ alkenyl; R₂₃ is halogen or —NHR₃₂, wherein R₃₂ is hydrogen, C₁₋₆ alkyl, C₂₋₆ alkenyl, C₃₋₈ cycloalkyl, —O—C₁₋₆ alkyl, —C(O)R^{A}, or —C(O)OR^{A}; R₂₄ is hydrogen, halogen, or —NHR₃₃; R₂₅ is hydrogen or —NHR₃₃; R₂₆ is hydrogen, halogen, C₁₋₆ alkyl, or C₂₋₆ alkenyl; R₂₇ is hydrogen, C₁₋₆ alkyl, C₃₋₈ cycloalkyl, —C(O)R^{A}, or —C(O)OR^{A}; R₂₈ is hydrogen, halogen, C₁₋₆ alkyl, or C₂₋₆ alkenyl; R₂₉ is hydrogen, halogen, C₁₋₆ alkyl, or C₂₋₆ alkenyl; R₃₀ is hydrogen, halogen, —NHR₃₃, C₁₋₆ alkyl, or C₂₋₆ alkenyl; each R₃₃ is independently selected from hydrogen, —C(O)R^{A}, or —C(O)OR^{A}; and each R^{A} is independently selected from C₁₋₆ alkyl, C₂₋₆ alkenyl, C₃₋₈ cycloalkyl, aryl, heteroaryl, heterocyclyl, aryl(C₁₋₆ alkyl), heteroaryl(C₁₋₆ alkyl), or heterocyclyl(C₁₋₆ alkyl). In other examples, B₁ is selected from

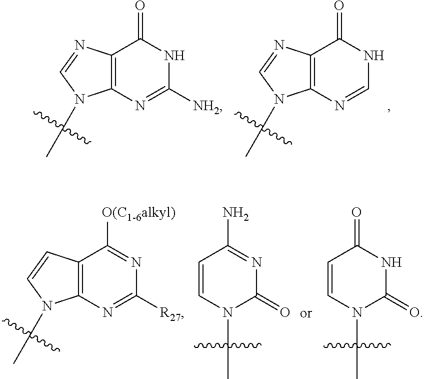

In some methods, R₂ is optionally substituted aryl. For example, R₂ is naphthyl or phenyl either of which is optionally substituted with 1-3 C₁₋₆ alkyl groups. In other examples, R₂ is unsubstituted phenyl.

In some methods, W is a bond, —S—, or —O—. For example, W is —S— or —O—.

In some methods, R₁₂ is a monocyclic saturated heterocyclic ring having 1-3 heteroatoms independently selected from N, O, or S, optionally substituted with 1-3 of R₁₃, wherein R₁₃ is oxo or an optionally substituted C₁₋₆ alkyl. For example, R₁₂ is oxazolidin-2-one optionally substituted with C₁₋₄ alkyl.

In some methods, —W—R₁₂ is

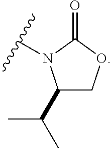 [SMILES: *N1C(=O)OC[C@H]1C(C)C]

In some methods, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

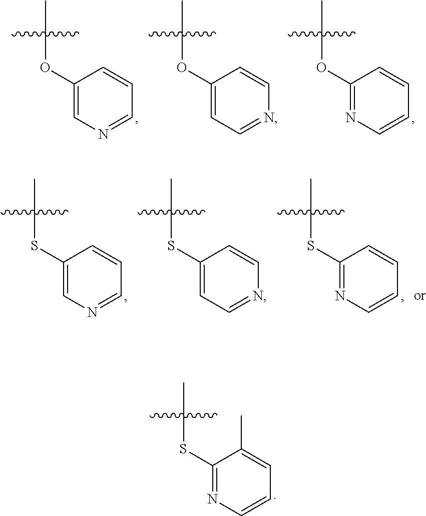

In some methods, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

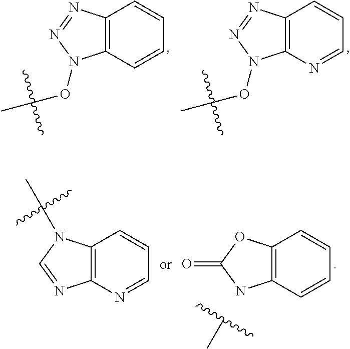

In some methods, —W—R₁₂ is

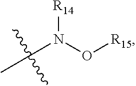

wherein R₁₄ and R₁₅ are each independently C₁₋₆ alkyl, cycloalkyl, or heteroalkyl, or R₁₄ and R₁₅, taken together with the heteroatoms to which they are attached, form a 6-10 membered heterocyclic ring optionally substituted with 1-3 of R₁₃.

In some methods, —W—R₁₂ is selected from

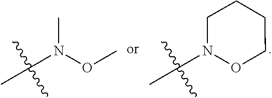

In some methods, the reaction of step ib) is performed in the presence of an acid, and the acid is a strong organic acid. For example, the acid is trifluoromethanesulfonic acid or methanesulfonic acid.

In some methods, the reaction of step ib) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step ib) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step ib) is performed in the presence of a metal salt of fluoroborate. In some examples, the metal salt of fluoroborate is silver tetrafluoroborate, silver hexafluorophosphate, or any combination thereof.

In some methods, the reaction of step ib) occurs in the presence of an organic solvent. For example, the organic solvent of step ib) is an aprotic organic solvent. In other examples, the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

In some methods, the reaction of step ib) is performed at a temperature of about 30° C. or less. For example, the reaction of step ib) is performed at a temperature of from about −20° C. to about 25° C.

Some methods further comprise step iib): reacting a compound of Formula C-1, wherein X^{A} is halogen, with H—W—R₁₂

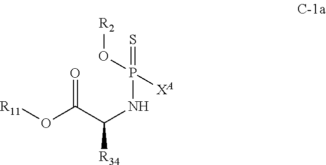

in the presence of a base to generate the compound of Formula B-1Ba.

In some methods, the base of step iib) is an amine base. For example, the base of step iib) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iib) is performed in the presence of an organic solvent. For example, the organic solvent of step iib) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iib) is performed at a temperature of about 30° C. or less. For example, the reaction of step iib) is performed at a temperature of from about −10° C. to about 25° C.

Some methods further comprise iiib): reacting a compound of Formula B-5A, wherein X^{B} is halogen, with a compound of Formula C-2

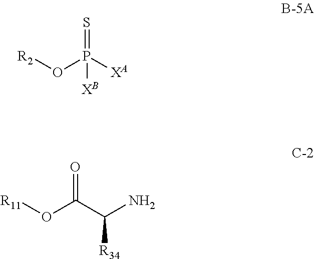

under nucleophilic substitution conditions to generate the compound of Formula C-la.

Another aspect of this application provides a method of preparing a compound of Formula IV

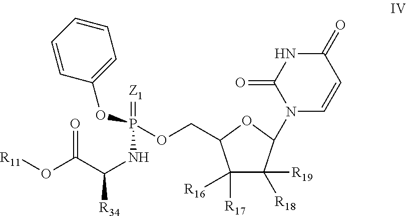

or a pharmaceutically acceptable salt thereof, having a diastereomeric purity of about 70% or greater (e.g., about 75% or greater or about 80% or greater), wherein Z₁ is S or O; R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; each of R₁₆, R₁₇, R₁₈, and R₁₉ is independently selected from hydrogen, —OH, halogen, azido, cyano, an optionally substituted C₁₋₆ alkyl, —OR₂₀ or —OC(O)R₂₁, or R₁₇ and R₁₈ are both oxygen atoms that are linked together by a carbonyl group; each of R₂₀, R₂₁, and R₂₂ is independently selected from hydrogen, optionally substituted C₁₋₆ alkyl or optionally substituted C₃₋₆ cycloalkyl; comprising step ic): reacting a compound of Formula A-2 and a compound of Formula B-1C,

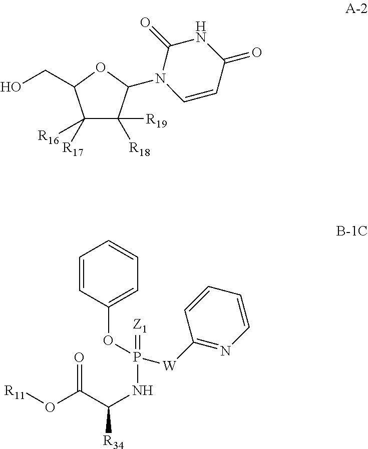

wherein W is —S— or —O—, in the presence of an acid or salt to generate the compound of Formula IV.

In some methods, the reaction of step ic) is performed in the presence of trifluoromethanesulfonic acid or methanesulfonic acid.

In some methods, the reaction of step ic) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step ic) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step ic) is performed in the presence of a metal salt of fluoroborate. In some examples, the metal salt of fluoroborate is silver tetrafluoroborate, silver hexafluorophosphate, or any combination thereof.

In some methods, the reaction of step ic) occurs in the presence of an organic solvent. In some examples, the organic solvent is an aprotic solvent. For example, the aprotic solvent is dichloromethane, 1,2-dichloroethane, chloroform, trifluorotoluene or 1,2-dichlorobenzene. In some examples, the aprotic solvent is 1,4-dioxane, tetrahydrofuran, 2-methyltetrahydrofuran, methyl-tert-butyl ether or cyclopentylmethyl ether. In other examples, the aprotic solvent is benzene, toluene or xylenes. And, in some examples, the aprotic solvent is sulfolane.

In some methods, the reaction of step ic) occurs in the presence of a mixture of solvents comprising a halogenated organic solvent and an aromatic hydrocarbon in 1:5 ratio. For example, the mixture of solvents comprises dichloromethane and toluene.

In some methods, the reaction of step ic) occurs in the presence of a mixture of solvents in the ratios of 1:1 to 4:1. For example, the mixture of solvents comprises dichloromethane and 1,4-dioxane.

In some methods, the reaction of step ic) occurs in the presence of a mixture of solvents comprising dichloromethane and sulfolane in 1:1 ratio.

In some methods, the reaction of step ic) is performed at a temperature of about 30° C. or less. For example, the reaction of step i) is performed at a temperature of from about −20° C. to about 25° C.

In some methods, the compound of Formula B-1C is a compound of Formula B-4B1 or B-4B2:

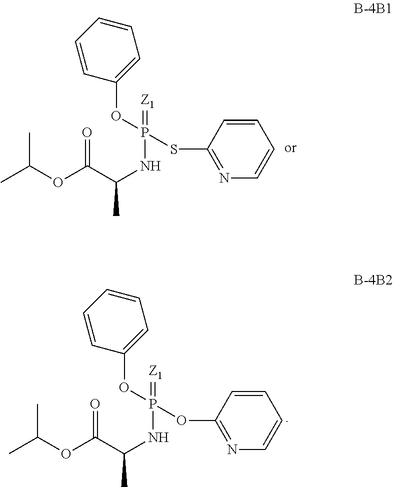

Some methods further comprise iic): reacting a compound of Formula C-3, wherein X^{A} is halogen,

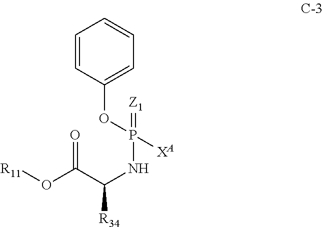

with

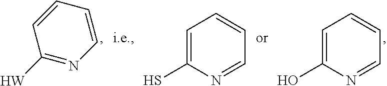

in the presence of a base to generate the compound of Formula B-1C.

In some methods, the base of step iic) is an amine base. For example, the base of step iic) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iic) is performed in the presence of an organic solvent. For example, the organic solvent of step iic) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iic) is performed at a temperature of about 30° C. or less. For example, the reaction of step iic) is performed at a temperature of from about −10° C. to about 25° C.

Some methods further comprise step iiic): reacting a compound of Formula B-5B, wherein X^{B} is halogen, with a compound of Formula C-2

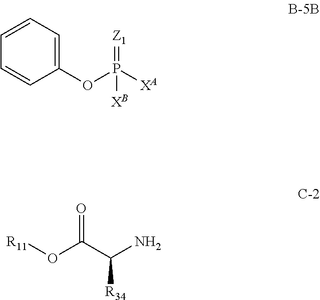

under nucleophilic substitution conditions to generate the compound of Formula C-3.

Another aspect of this application provides a method of preparing a compound of Formula IVa

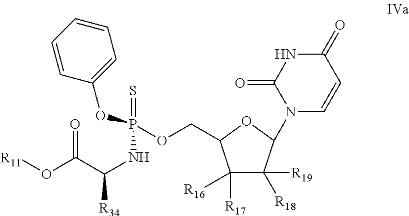

or a pharmaceutically acceptable salt thereof, having a diastereomeric purity of about 70% or greater (e.g., about 75% or greater or about 80% or greater), wherein R₃₄ is C₁₋₆ alkyl, halo-C₁₋₆ alkyl, C₃₋₈ cycloalkyl, aryl, or aryl(C₁₋₆ alkyl); R₁₁ is hydrogen, C₁₋₆ alkyl, C₃₋₈ cycloalkyl, aryl, aryl(C₁₋₆ alkyl), or halo-C₁₋₆ alkyl; each of R₁₆, R₁₇, R₁₈, and R₁₉ is independently selected from hydrogen, —OH, halogen, azido, cyano, an optionally substituted C₁₋₆ alkyl, —OR₂₀ or —OC(O)R₂₁, or R₁₇ and R₁₈ are both oxygen atoms that are linked together by a carbonyl group; each of R₂₀ and R₂₁ is independently selected from hydrogen, optionally substituted C₁₋₆ alkyl or optionally substituted C₃₋₆ cycloalkyl; comprising step ic): reacting a compound of Formula A-2 and a compound of Formula B-1Ca,

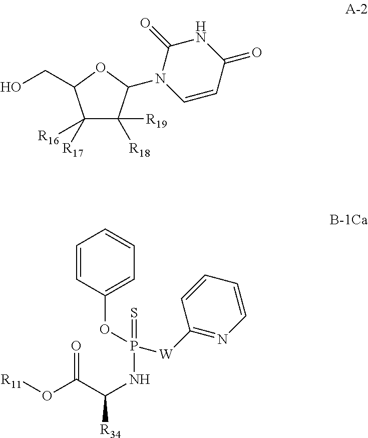

wherein W is —S— or —O—, in the presence of an acid or salt to generate the compound of Formula IV.

In some methods, the reaction of step ic) is performed in the presence of trifluoromethanesulfonic acid or methanesulfonic acid.

In some methods, the reaction of step ic) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step ic) is performed in the presence of a salt of acetate. In some examples, the salt of acetate is palladium(II) acetate, copper(I) acetate, tetrakis(acetonitrile)copper(I) hexafluorophosphate, or any combination thereof

In some methods, the reaction of step ic) is performed in the presence of a metal salt of fluoroborate. In some examples, the metal salt of fluoroborate is silver tetrafluoroborate, silver hexafluorophosphate, or any combination thereof.

In some methods, the reaction of step ic) occurs in the presence of an organic solvent. In some examples, the organic solvent is an aprotic solvent. For example, the aprotic solvent is dichloromethane, 1,2-dichloroethane, chloroform, trifluorotoluene or 1,2-dichlorobenzene. In some examples, the aprotic solvent is 1,4-dioxane, tetrahydrofuran, 2-methyltetrahydrofuran, methyl-tert-butyl ether or cyclopentylmethyl ether. In other examples, the aprotic solvent is benzene, toluene or xylenes. And, in some examples, the aprotic solvent is sulfolane.

In some methods, the reaction of step ic) occurs in the presence of a mixture of solvents comprising a halogenated organic solvent and an aromatic hydrocarbon in 1:5 ratio. For example, the mixture of solvents comprises dichloromethane and toluene.

In some methods, the reaction of step ic) occurs in the presence of a mixture of solvents in the ratios of 1:1 to 4:1. For example, the mixture of solvents comprises dichloromethane and 1,4-dioxane.

In some methods, the reaction of step ic) occurs in the presence of a mixture of solvents comprising dichloromethane and sulfolane in 1:1 ratio.

In some methods, the reaction of step ic) is performed at a temperature of about 30° C. or less. For example, the reaction of step i) is performed at a temperature of from about −20° C. to about 25° C.

In some methods, the compound of Formula B-1Ca is a compound of Formula B-4B1a or B-4B2a:

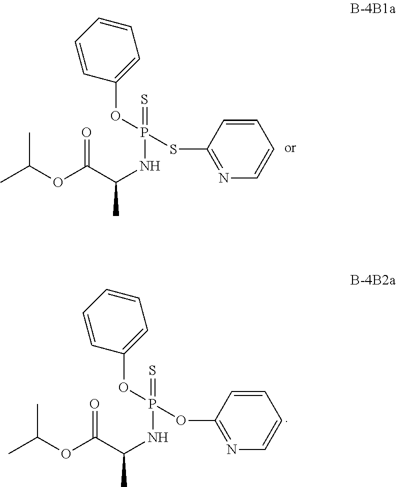 [SMILES: CC(C)OC(=O)[C@H](C)NP(=S)(Oc1ccccc1)Sc1ccccn1 ; CC(C)OC(=O)[C@H](C)NP(=S)(Oc1ccccc1)Oc1ccccn1]

Some methods further comprise iic): reacting a compound of Formula C-3a, wherein X^{A} is halogen,

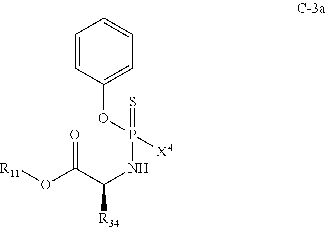

with

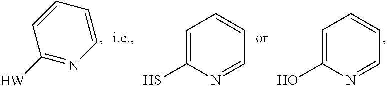

in the presence of a base to generate the compound of Formula B-1C.

In some methods, the base of step iic) is an amine base. For example, the base of step iic) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

In some methods, the reaction of step iic) is performed in the presence of an organic solvent. For example, the organic solvent of step iic) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iic) is performed at a temperature of about 30° C. or less. For example, the reaction of step iic) is performed at a temperature of from about −10° C. to about 25° C.

Some methods further comprise step iiic): reacting a compound of Formula B-5Ba, wherein X^{B} is halogen, with a compound of Formula C-2

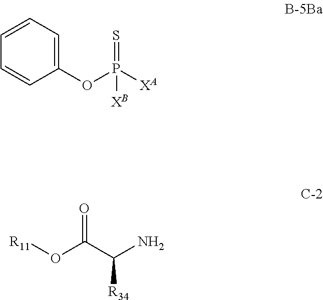

under nucleophilic substitution conditions to generate the compound of Formula C-3a.

Another aspect of this application provides a method of preparing a compound of Formula V:

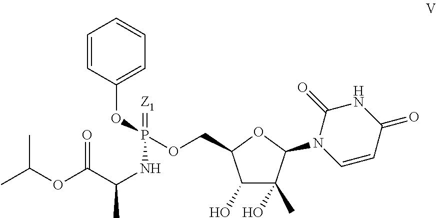

or a pharmaceutically acceptable salt thereof, having a diastereomeric purity of about 75% or greater, wherein Z₁ is O or S; comprising step id): reacting a compound of Formula A-3 and a compound of Formula B-4B1

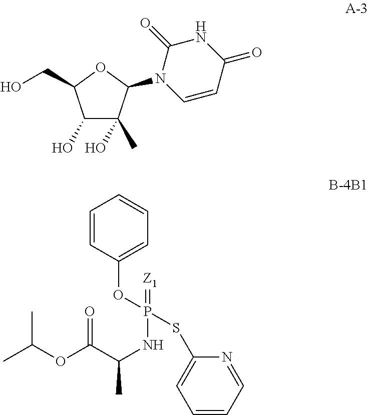

in the presence of an acid or salt to generate the compound of Formula V.

In some embodiments, Z′ is O. In other embodiments, Z¹ is S.

In some methods, the reaction of step id) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step id) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step id) is performed in the presence of a metal salt of fluoroborate. In some examples, the metal salt of fluoroborate is silver tetrafluoroborate, silver hexafluorophosphate, or any combination thereof.

In some methods, the reaction of step id) is performed in the presence of an organic solvent. For example, the organic solvent is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step id) is performed at a temperature of about 30° C. or less.

Another aspect of this application provides a method of preparing a compound of Formula Va

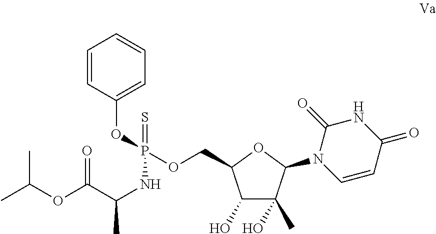 [SMILES: CC(C)OC(=O)[C@H](C)N[P@](=S)(OC[C@H]1O[C@@H](n2ccc(=O)[nH]c2=O)[C@](C)(O)[C@@H]1O)Oc1ccccc1]

or a pharmaceutically acceptable salt thereof, having a diastereomeric purity of about 75% or greater; comprising step id): reacting a compound of Formula A-3 and a compound of Formula B-4B1a

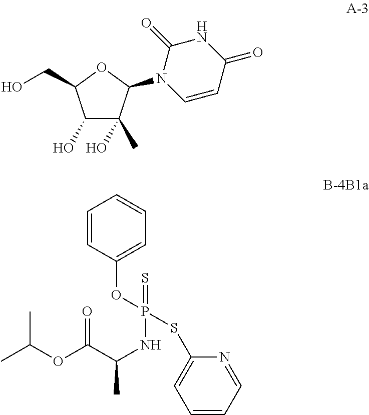 [SMILES: C[C@@]1(O)[C@H](O)[C@@H](CO)O[C@H]1n1ccc(=O)[nH]c1=O ; CC(C)OC(=O)[C@H](C)NP(=S)(Oc1ccccc1)Sc1ccccn1]

in the presence of an acid or salt to generate the compound of Formula Va.

In some methods, the reaction of step id) is performed in the presence of a metal salt, and the salt is a metal salt of trifluoromethanesulfonate. In some examples, the metal salt of trifluoromethanesulfonate is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, or any combination thereof.

In some methods, the reaction of step id) is performed in the presence of a salt of acetate. In some examples, the salt of acetate is palladium(II) acetate, copper(I) acetate, tetrakis(acetonitrile)copper(I) hexafluorophosphate, or any combination thereof.

In some methods, the reaction of step id) is performed in the presence of a metal salt of fluoroborate. In some examples, the metal salt of fluoroborate is silver tetrafluoroborate, silver hexafluorophosphate, or any combination thereof.

In some methods, the reaction of step id) is performed in the presence of an organic solvent. For example, the organic solvent is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step id) is performed at a temperature of about 30° C. or less.

Another aspect of this application provides a method of preparing a compound of Formula V-2

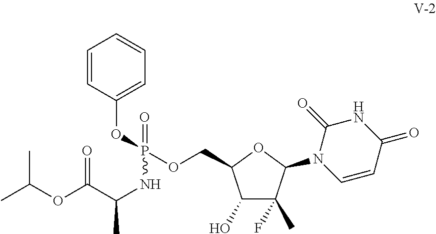

or a pharmaceutically acceptable salt thereof, having a diastereomeric purity of about 75% or greater; comprising step id): reacting a compound of Formula A-4A and a compound of Formula 8

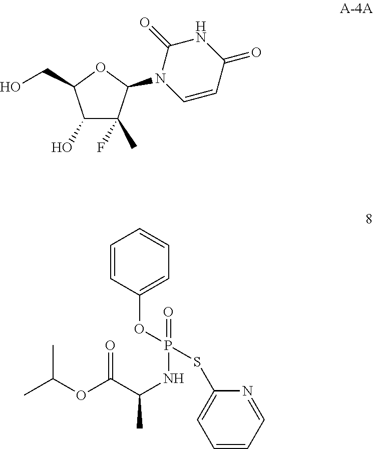 [SMILES: C[C@@]1(F)[C@H](O)[C@@H](CO)O[C@H]1n1ccc(=O)[nH]c1=O ; CC(C)OC(=O)[C@H](C)NP(=O)(Oc1ccccc1)Sc1ccccn1]

in the presence of an acid or salt to generate the compound of Formula V-2.

In some methods, the reaction of step id) is performed in the presence of trifluoromethanesulfonic acid or methanesulfonic acid.

Another aspect of this application provides a method of preparing a compound of Formula B-1B:

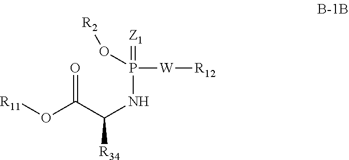

wherein Z₁ is S or O; R₂ is optionally substituted aryl or optionally substituted heteroaryl; W is —O— or —S—; R₁₂ is a 6-10-membered mono- or bicyclic saturated, partially unsaturated, or fully unsaturated heterocyclic ring having 1-4 heteroatoms independently selected from N, O, or S, wherein R₁₂ is optionally substituted with 1-2 of C₁₋₆ alkyl; R₃₄ is C₁₋₆ alkyl, halo-C₁₋₆ alkyl, C₃₋₈ cycloalkyl, aryl, or aryl(C₁₋₆ alkyl); and R₁₁ is hydrogen, C₁₋₆ alkyl, C₃₋₈ cycloalkyl, aryl, aryl(C₁₋₆ alkyl), or halo-C₁₋₆ alkyl; comprising step iv): reacting a compound of Formula C-1, wherein X^{A} is halogen,

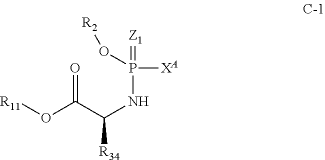

with H—W—R₁₂ in the presence of a base to generate the compound of Formula B-1B.

In some methods, Z₁ is S.

In some embodiments, R₂ is optionally substituted aryl. For example, R₂ is phenyl or naphthyl optionally substituted with 1-3 of C₁₋₆ alkyl. In other examples, R₂ is unsubstituted phenyl.

In some methods, R₁₂ is a monocyclic saturated heterocyclic ring having 1-3 heteroatoms independently selected from N, O, or S, optionally substituted with 1-3 of R₁₃, wherein R₁₃ is oxo or an optionally substituted C₁₋₆ alkyl. For example, R₁₂ is oxazolidin-2-one, optionally substituted with C₁₋₄ alkyl.

In some methods, —W—R₁₂ is

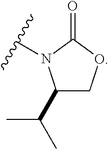 [SMILES: *N1C(=O)OC[C@H]1C(C)C]

In some methods, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some methods, —W—R₁₂ is selected from

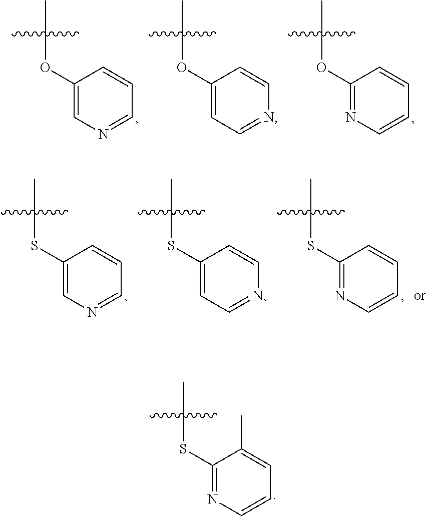

In some methods, R₁₂ is an 8-10-membered bicyclic heteroaryl having 1-5 heteroatoms independently selected from N, O, or S, optionally substituted with 1-4 of R₁₃, wherein R₁₃ is an optionally substituted C₁₋₆ alkyl.

In some methods, —W—R₁₂ is selected from

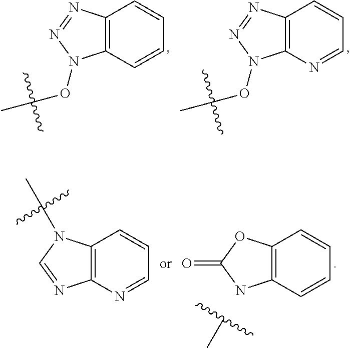

In some methods, —W—R₁₂ is

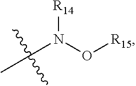

wherein R₁₄ and R₁₅ are each independently C₁₋₆ alkyl, cycloalkyl, or heteroalkyl, or R₁₄ and R₁₅, taken together with the heteroatoms to which they are attached, form a 6-10 membered heterocyclic ring optionally substituted with 1-3 of R₁₃.

In some methods, —W—R₁₂ is selected from

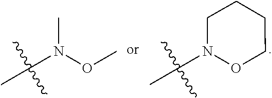

In some embodiments, R₃₄ is C₁₋₆ alkyl or halo-C₁₋₆ alkyl. For example, R₃₄ is methyl, ethyl, propyl, iso-propyl, butyl, sec-butyl, or tert-butyl.

In some embodiments, R₁₁ is hydrogen, C₁₋₆ alkyl, or C₃₋₈ cycloalkyl. For example, R₁₁ is C₁₋₆ alkyl. In other examples, R₁₁ is methyl, ethyl, propyl, iso-propyl, butyl, sec-butyl, or tert-butyl, any of which is optionally substituted with 1-3 halo.

In some methods, the base of step iv) is an amine base. For example, the base of step iv) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof. For example, the base of step iv) is 1,4-diazabicyclo[2.2.2]octane.

In some methods, the reaction of step iv) is performed in the presence of an organic solvent. For example, the organic solvent of step iv) is an aprotic organic solvent. In other examples, the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

In some methods, the reaction of step iv) is performed at a temperature of about 30° C. or less. For example, the reaction of step iv) is performed at a temperature of from about −10° C. to about 25° C.

Some methods further comprise step v): reacting a compound of Formula BB, wherein X^{B} is halogen, with a compound of Formula C-2

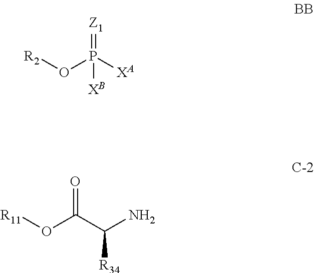

under nucleophilic substitution conditions to generate the compound of Formula C-1.

In some methods, neutralizing the charge on the thiophosphate group may facilitate the penetration of the cell membrane by a compound of Formula I or their pharmaceutically acceptable salts (including the compound of Formulae II, II-1, III, IV and V), or a pharmaceutically acceptable salt of the aforementioned) by making the compound more lipophilic compared to thionucleoside having a comparable structure with one or more charges present on the thiophosphate. Once absorbed and taken inside the cell, the groups attached to the thiophosphate can be easily removed by esterases, proteases, or other enzymes. In some embodiments, the groups attached to the thiophosphate can be removed by simple hydrolysis. Inside the cell, the thio-monophosphate thus released may then be metabolized by cellular enzymes to the thio-diphosphate or the active thio-triphosphate. In some embodiments, the phosphorylation of a thio-monophosphate of a compound of Formula I, or a pharmaceutically acceptable slat thereof, can be stereoselective. For example, a thiomonophosphate of a compound of Formula V (including both the diastereomers of Formula V) can be phosphorylated to give an alpha-thiodiphosphate and/or an alpha-thiotriphosphate compound that can be enriched in the (R) or (S) diastereomer with respect to the 5′-O-phosphorus atom

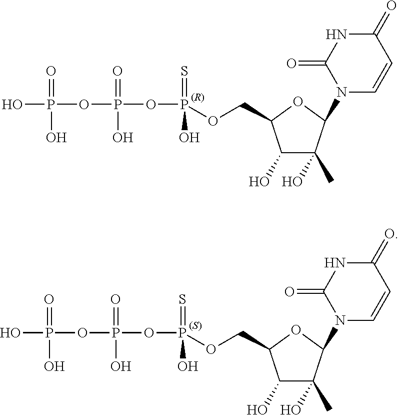

For example, one of the (R) and (S) configuration with respect to the 5′-O-phosphorous atom of the alpha-thiodiphosphate and/or the alpha-thiotriphosphate compound can be present in an amount ≧50%, ≧75%, ≧90%, ≧95% or ≧99% compared to the amount of the other of the (R) or (S) configuration with respect to the 5′-O-phosphorous atom. In some embodiments, phosphorylation of a compound of Formula I, or a pharmaceutically acceptable slat thereof, can result in the formation of a compound that has the (R)-configuration a the 5′-O-phosphorus atom. In some embodiments, phosphorylation of a compound of Formula I, or pharmaceutically acceptable slat thereof, can result in the formation of a compound that has the (S)-configuration at the 5′-O-phosphorus atom.

### IV. COMPOUNDS & INTERMEDIATES

One aspect of the present application provides a compound of Formula B-1B:

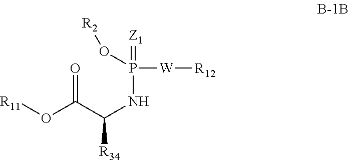

wherein Z₁ is S or O; R₂ is optionally substituted aryl or optionally substituted heteroaryl; W is a bond, —O— or —S—; R₁₂ is a 6-10-membered mono- or bi-cyclic saturated, partially unsaturated, or fully unsaturated heterocyclic ring having 1-4 heteroatoms independently selected from N, O, or S, wherein R₁₂ is optionally substituted with 1-3 of R₁₃ and R₁₃ is oxo or an optionally substituted C₁₋₆ alkyl; R₃₄ is C₁₋₆ alkyl, halo-C₁₋₆ alkyl, C₃₋₈ cycloalkyl, aryl, or aryl(C₁₋₆ alkyl); and R₁₁ is hydrogen, C₁₋₆ alkyl, C₃₋₈ cycloalkyl, aryl, aryl(C₁₋₆ alkyl), or halo-C₁₋₆ alkyl.

In some embodiments, R₂ is optionally substituted aryl. For example, R₂ is phenyl or naphthyl optionally substituted with 1-3 of C₁₋₆ alkyl. In other examples, R₂ is unsubstituted phenyl.

In some embodiments, R₁₂ is a monocyclic saturated heterocyclic ring having 1-3 heteroatoms independently selected from N, O, or S, optionally substituted with 1-3 of R₁₃, wherein R₁₃ is an optionally substituted C₁₋₆ alkyl. For example, R₁₂ is oxazolidin-2-one, either of which is optionally substituted with C₁₋₄ alkyl.

In some embodiments, —W—R₁₂ is

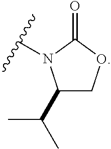 [SMILES: *N1C(=O)OC[C@H]1C(C)C]

In some embodiments, R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl. For example, R₁₂ is pyridine or pyrimidine, either of which is optionally substituted with C₁₋₆ alkyl.

In some embodiments, —W—R₁₂ is selected from

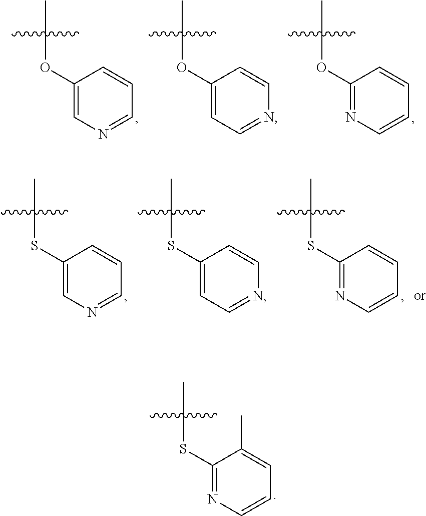

In some embodiments, R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl.

In some embodiments, —W—R₁₂ is selected from

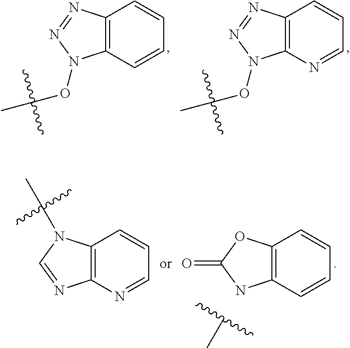

In some embodiments, —W—R₁₂ is

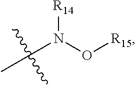

wherein R₁₄ and R₁₅ are each independently C₁₋₆ alkyl, cycloalkyl, or heteroalkyl, or R₁₄ and R₁₅, taken together with the heteroatoms to which they are attached, form a 6-10 membered heterocyclic ring optionally substituted with 1-3 of R₁₃.

In some embodiments, —W—R₁₂ is selected from

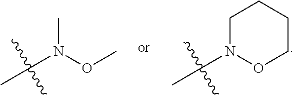

In some embodiments, R₃₄ is C₁₋₆ alkyl or halo-C₁₋₆ alkyl. For example, R₃₄ is methyl, ethyl, propyl, iso-propyl, butyl, sec-butyl, or tert-butyl, any of which is optionally substituted with 1-3 halo.

In some embodiments, R₁₁ is hydrogen, C₁₋₆ alkyl, or C₃₋₈ cycloalkyl. For example, R₁₁ is C₁₋₆ alkyl. In other examples, R₁₁ is methyl, ethyl, propyl, iso-propyl, butyl, sec-butyl, or tert-butyl.

In some embodiments, the compound of Formula B-1B is a compound of Formula B-1Ba

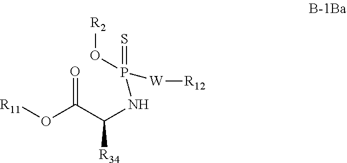

wherein R₂, W, R₁₁, R₁₂, and R₃₄ are as defined above.

### V. SYNTHETIC SCHEMES

The synthetic routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

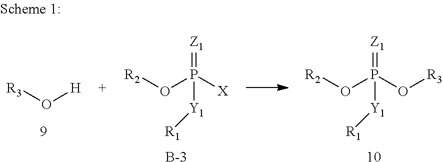

In Scheme 1, compounds of Formulae 9 and B-3 undergo a nucleophilic substitution reaction in the presence of an acid, a salt, a base, or a Grignard reagent to generate the compound of Formula 10.

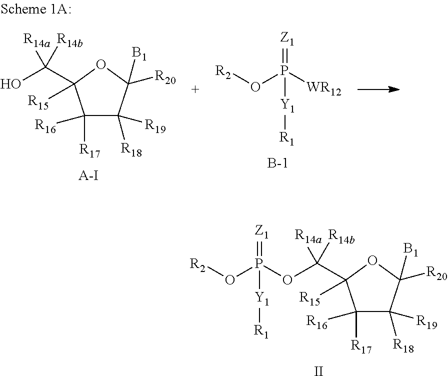

In Scheme 1A, the starting materials that undergo nucleophilic substitution are compounds of Formulae A-1 and B-1A. In some methods, the reaction between the compound of Formula A-1 and the compound of Formula B-1A is conducted in the presence of a strong acid or salt. An example of a suitable acid is trifluoromethanesulfonic acid. Examples of suitable salts are sodium trifluoromethanesulfonate, potassium trifluoromethanesulfonate and silver trifluoromethanesulfonate. This reaction can be performed either at room temperature or about 30° C.

In scheme 1A, the diastereomerically enriched compounds of Formulae B-1B, B-1C, or B-4B1 can be substituted for the compound of Formula B-1A to react with the compound of Formula A to give diastereomerically enriched compounds of Formula I (e.g., compounds of Formulae III, IV or V) in the presence of a strong acid or salt. A reaction between a compound of Formulae B-1B, B-1C, or B-4B1 (diastereomerically enriched with a diastereomeric ratio of at least 7:1) and a compound of Formula A (for example, Formula A-1) as described herein can provide a compound of Formula I that can be ≧70%, ≧85%, ≧90%, ≧95% enriched in one diastereomer with respect to the phosphorous.

The compounds of Formulae B-1B, B-1C, or B-4B1 can be synthesized from heterocyclic phenol or thiophenol and Formula B (where X=Cl) in the presence of a base in aprotic solvents as shown in general Scheme 1A. The reaction can proceed diastereoselectively. Bases like N-methyl imidazole, 4-(dimethylamino)pyridine, 3,4-lutidine, 4-methoxypyridine, N-methylpyrrolidine and bicycle[2.2.2]octane can give diastereoselectivity in the range of 2:1 to 7.1:1. In some embodiments, the use of 1,4-diazabicyclo[2.2.2]octane can give higher diastereoselectivity compared to triethylamine. Additionally the diastereoselective reaction can take place in a solvent. Suitable solvents include, but are not limited to aprotic solvents. Examples of polar aprotic solvents include toluene, dichloromethane, ethyl acetate, iso-propyl acetate, tert-butyl acetate, methyl isobutyl ketone, diethyl ether, 1,4-dioxane, tetrahydrofuran, 2-methyltetrahydrofuran, methyl tert-butyl ether, cyclopentyl methyl ether, 2-butanone and acetonitrile.

Advantageously, using the synthesis shown in Scheme 1A, it is not necessary to protect one more hydroxy groups (such as the hydroxy groups attached to the T-position and the 3′-position of the pentose ring) and/or one or more amine groups (for example, Formula B-1 wherein B₁ is heterocyclic moiety) prior to coupling of the compound of Formula A to the compound of Formula B. Protecting groups, for example, protecting groups on the oxygens at the 2′-position and/or 3′-position of the pentose ring, and/or on the amine of the uracil (A-3), can optionally be used to minimize the formation of undesirable amounts of side reaction byproduct(s). However, use of protecting groups increase the number of steps in the formation of the desired product and can decrease the overall yield of the desired product. The synthesis shown in Scheme 1A can result in a higher yield of the desired product and/or fewer reaction steps, as protection and deprotection steps are not included.

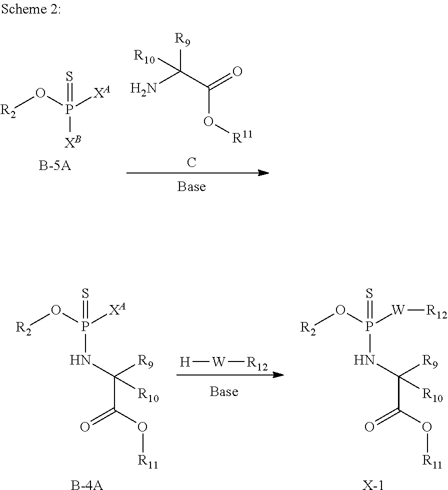

In Scheme 2, the preparation of intermediate X-1 can be diastereoselective. The compound of Formula B-5A reacts with the compound of Formula C under basic conditions to generate the compound of Formula B-4A, which is treated with H—W—R₁₂ under basic conditions to generate the compound of Formula X-1. The compound of Formula X-1 can be prepared with a high degree of diastereoselectivity depending on the type of base used in the last step of the reaction.

Various methods are known to those skilled in the art for isolating the final compound (e.g., a compound of Formula I). In some embodiments, the final compound can be isolated by filtration.

### VI. EXAMPLES

Additional embodiments are disclosed in further detail in the following examples, which are not in any way intended to limit the scope of the claims.

### Example 1

### Preparation of (2S)-isopropyl 2-((chloro(phenoxy)phosphorothioyl)amino)propanoate (Compound 3)

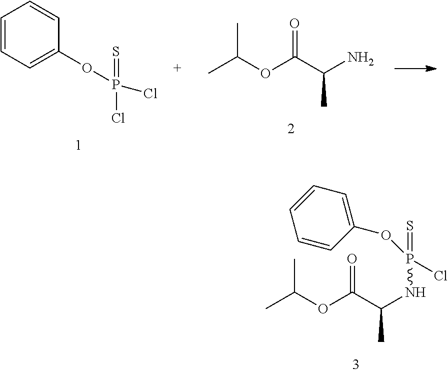

Method A1:

To a 20 L jacketed reactor, equipped with reflux condenser, N₂ inlet, temperature controller, and thermocouple with reaction monitoring software, was charged with (S)-isopropyl 2-aminopropanoate hydrochloride (compound 2, 620.19 g, 3.70 mol, 1.05 eq.), dichloromethane (8.0 L) and O-phenylphosphorodichlororidothioate (compound 1, 800 g, 3.52 mol, 1.0 eq.). The mixture was cooled to 0° C. Triethylamine (749 g, 7.40 mol, 2.1 eq.) was added over 3 to 5 hrs while maintaining the temperature below 0° C. The mixture was stirred at 0° C. for 2 hr, warmed to 20° C. over a period of ˜5 hrs and stirred for 16 hrs. A sample was tested using an in process control and conversion was shown to be 99.5%.

The mixture was concentrated to 2.4 to 3.2 L, and then charged with MTBE (8 L). The mixture was stirred for ˜30 min. The slurry was filtered. The wet cake was washed with MTBE (1.6 L) to obtain a clear solution. The solution was filtered through a pad of silica gel and washed with MTBE (2.4 L). The combined organic solution was concentrated under vacuum to give compound 3 as colorless oil. The product was used in the next step without further purification.

Method B1:

To a 100 mL jacketed reactor, equipped with reflux condenser, N₂ inlet, temperature controller, and thermocouple coupled with reaction monitoring software, was charged with (S)-isopropyl-2-aminopropanoate methanesulfonic acid (compound 2, 1.73 g, 0.013 mol, 1.05 eq.), dichloromethane (28.5 mL) and O-phenylphosphorodichloridothioate (compound 1, 2.85 g, 0.013 mol, 1 eq.). The mixture was cooled to 0° C. Triethylamine (2.67 g, 2.1 eq.) was added over 3 hrs while maintaining the temperature below 0° C. The mixture was stirred at 0° C. for 2 hrs, warmed to 20° C. over a period of 1 hr and stirred for 16 hrs. A sample was tested using an in process control and conversion was shown to be 99.5%.

The mixture was concentrated to 9 to 12 mL, was charged with MTBE (28.5 mL), and stirred for ˜30 minutes. The slurry was filtered. The wet cake was washed with MTBE (6 mL) to obtain a clear solution. The solution was filtered through a pad of silica gel and washed with MTBE (9 mL). The combined organic solution was concentrated under vacuum to give compound 3 as colorless oil. The product was used in the next step without further purification.

Compound 3 resulting from Methods B1 and C1 was found to be 1:1 diastereomeric mixture compounds.

### Example 2A

### Preparation of (2S)-isopropyl 2-((phenoxy(pyridin-2-ylthio)phosphorothioyl)amino)propanoate (Compound B-4B1a)

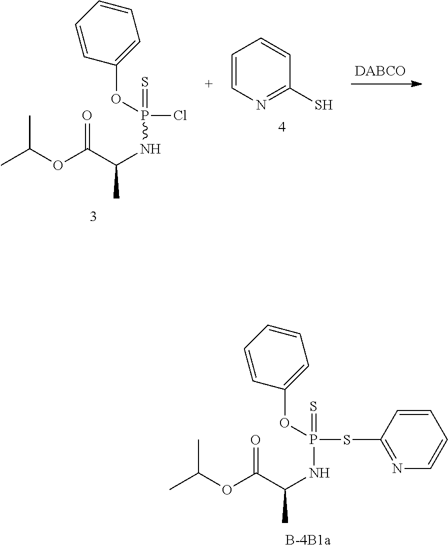

To a 50 L glass reactor equipped with a mechanical stirrer, reflux condenser, N₂ inlet, temperature controller, and thermocouple coupled with reaction monitoring software, was charged with 1,4-diazabicyclo[2.2.2]octane (DABCO) (2.61 kg, 1.3 eq.), methyl tert-butyl ether (MTBE) (12 L) and 2-mercaptopyridine (1.02 kg, 1.3 eq.) at 20° C. The mixture was cooled to 0.9° C. To it (25)-isopropyl 2-((chloro(phenoxy)phosphorothioyl)amino)propanoate (compound 3, 2.27 kg, 1 eq.) dissolved in MTBE (2.56 L) was added over 30 minutes. The charge tank and lines were rinsed with 1 L of MTBE. The internal temperature of the reaction was adjusted to 5° C. and kept at 5° C. for 3 hrs. The reaction was heated to 30° C. over 5 hrs and held at 30° C. for 14.5 hrs. The reaction was cooled to 2.3° C. and 1N hydrochloric acid (11.1 L) was added over 30 minutes while maintaining the internal temperature <15° C. The batch temperature was adjusted to 22.3° C. and maintained without stirring. The phases were allowed to separate and the lower aqueous layer was removed. To the reaction mixture 1N hydrochloric acid (11.1 L) was added and the resulting mixture was stirred for 30 minutes. After stopping the stirring, the phases were again allowed to separate and the lower aqueous layer was removed. The reaction mixture was washed two times with 8% aqueous sodium bicarbonate solution (11.1 L) followed by washing with 5% aqueous sodium chloride solution (10 L). The organic solution was transferred to another vessel. The reactor was rinsed with toluene (4 L) and the combined organic layer was concentrated under reduced pressure using rotary evaporator at a bath temperature of 35° C. The concentrate was dissolved in toluene (3.5 L) and further concentrated under reduced pressure using rotary evaporator at 50° C. to give compound B-4B1 as a yellow oil. The compound was used without any further purification.

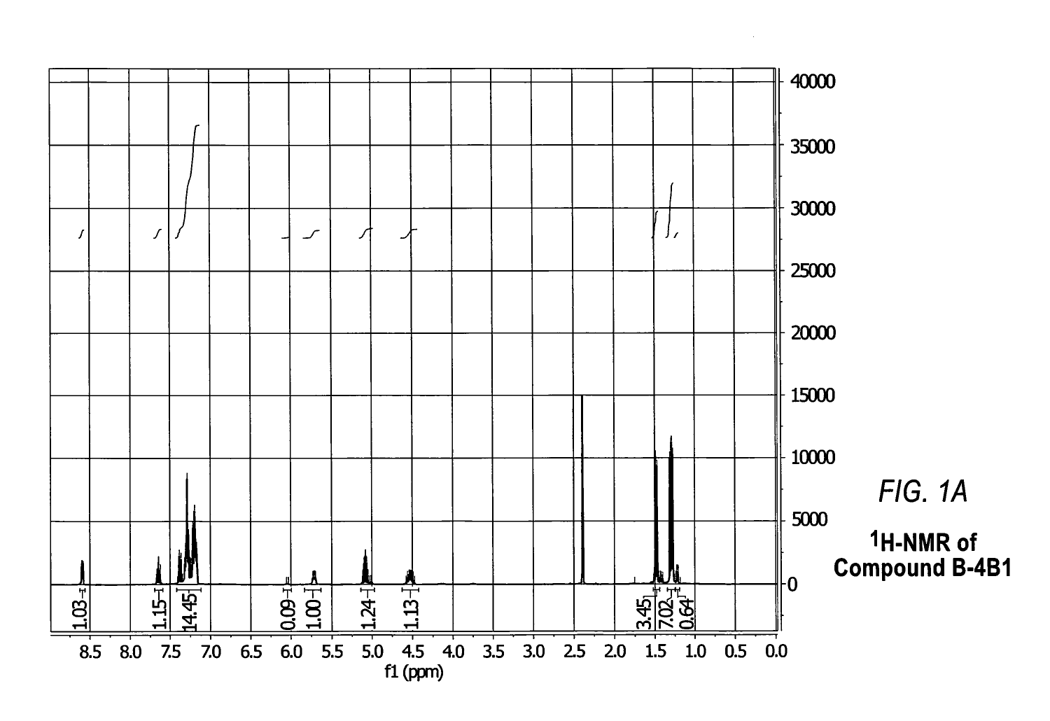

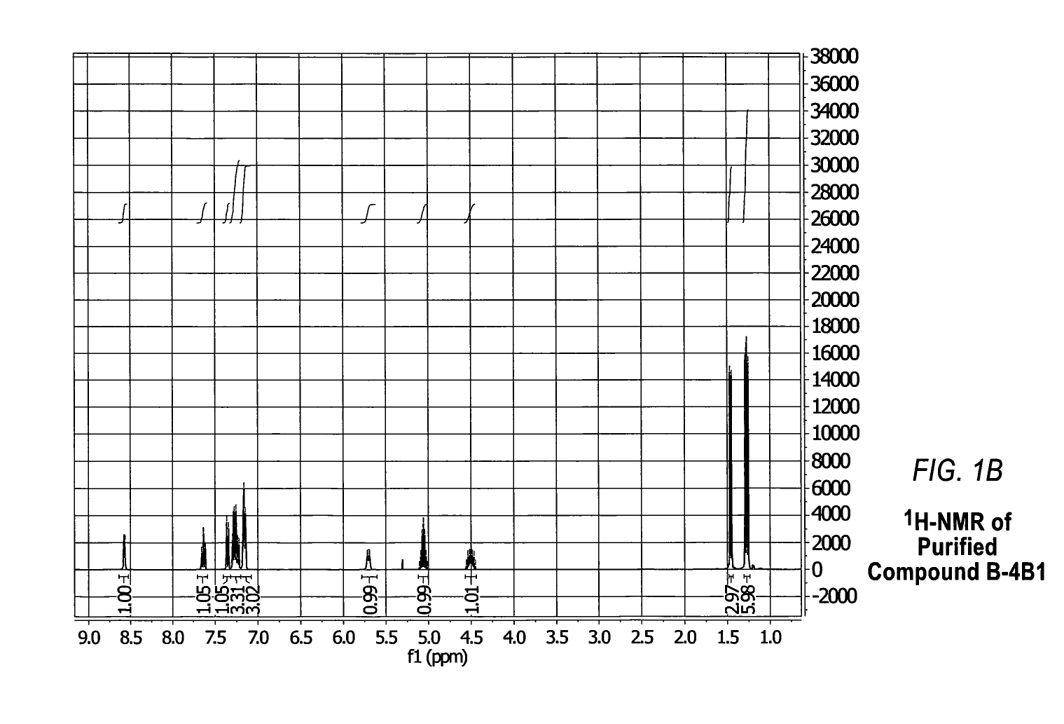

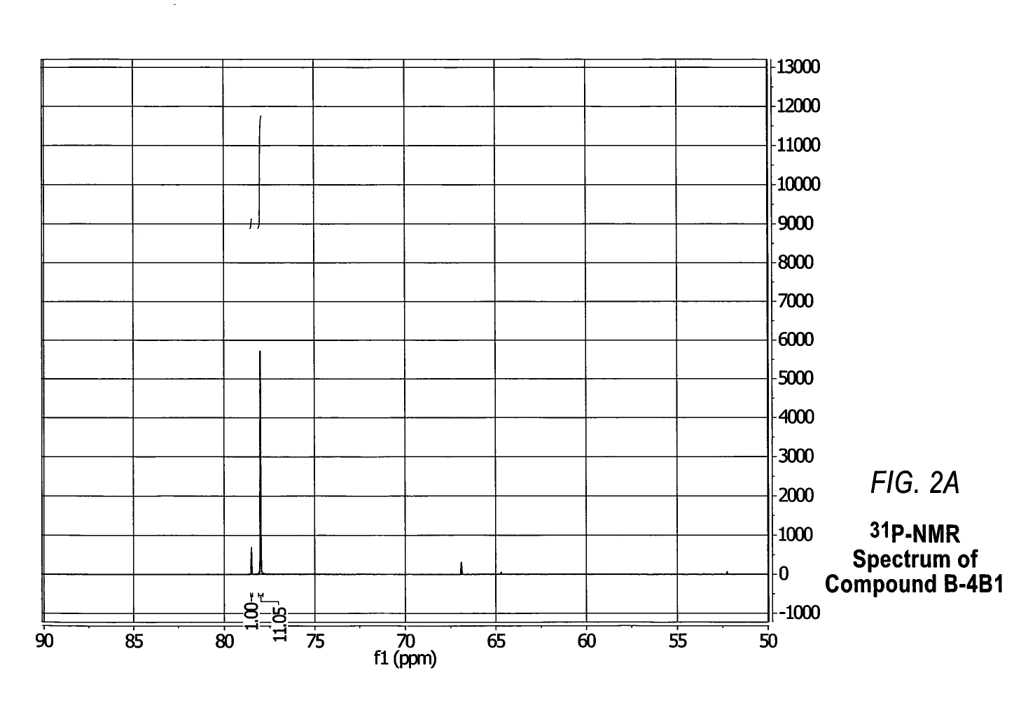

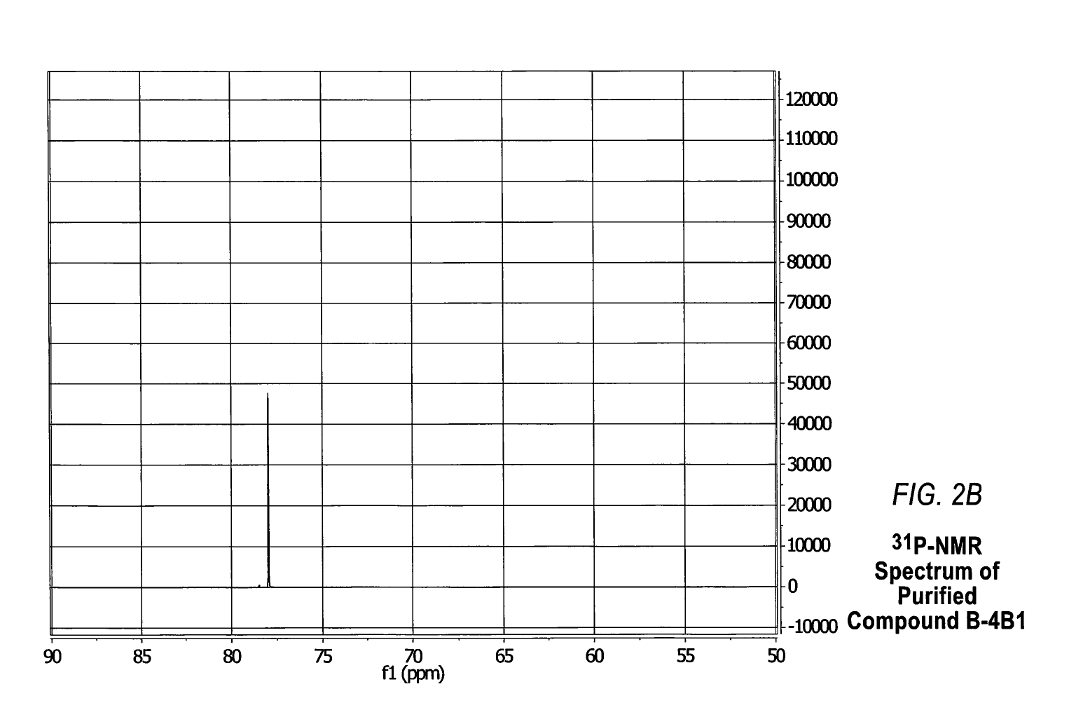

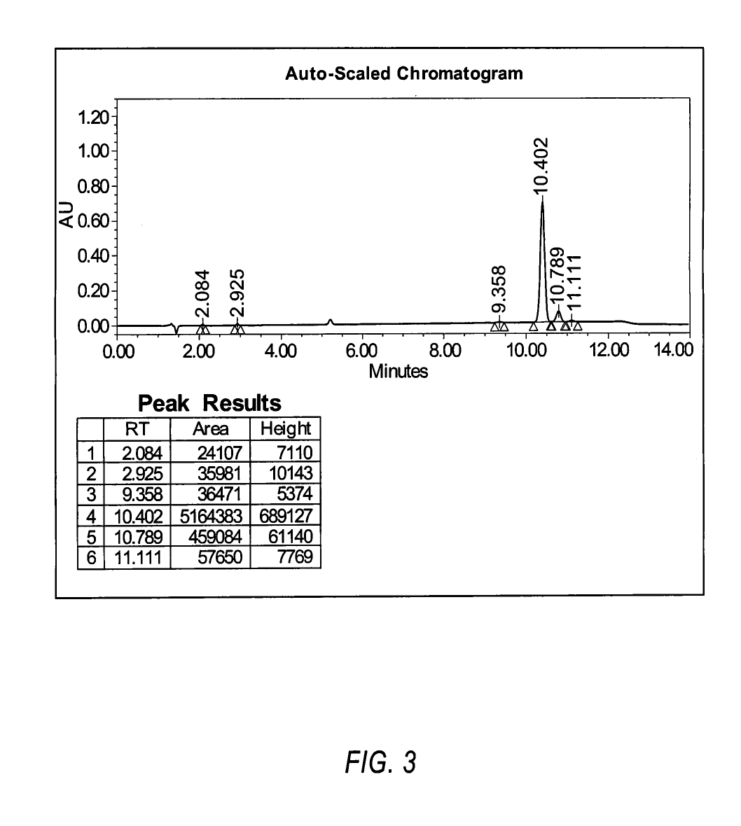

FIG. 1A is a ¹H NMR spectrum of the product (compound B-4B1), whereas FIG. 1B is a ¹H NMR spectrum of the purified product (compound B-4B1). FIG. 2A is a ³¹P NMR spectrum of the product (compound B-4B1), whereas FIG. 2B is a ³¹P NMR spectrum of the purified product (compound B-4B1). A HPLC chromatogram of the reaction is provided in FIG. 3. HPLC analysis of compound B-4B1 resulting from the above reaction was found to be 11.25:1 diastereomeric mixture compounds.

### Example 2B

### Preparation of (S)-isopropyl 2-(((R)-phenoxy(pyridin-2-yloxy)phosphoryl)amino)propanoate (Compound B-4B3)

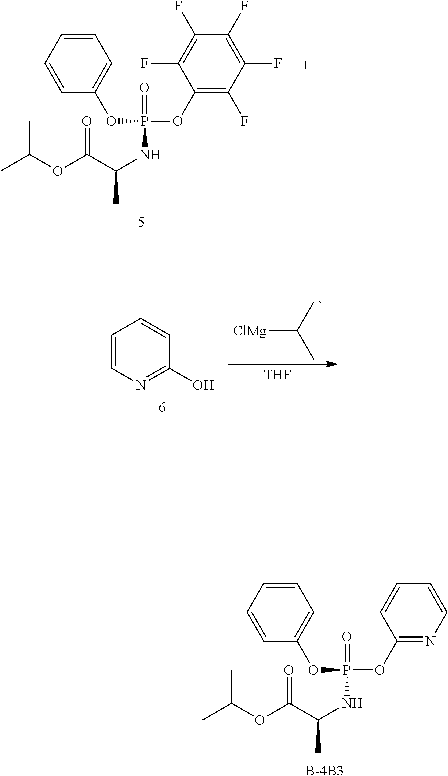

2-hydroxypyridine (6) (50.4 mg, 0.53 mmol, 1.2 equiv) was charged to a nitrogen purged vial followed by anhydrous THF (1 mL). The stirring mixture was cooled to 0° C. and charged with isopropylmagnesium chloride (2 M, 287 μL, 0.57 mmol, 1.3 equiv) over 7 min. The mixture was stirred at 0° C. for 15 min then warmed to 23° C. and stirred for an additional 40 min. The mixture was cooled again to 0° C. and charged with a solution of pentafluorophenyl derivative 5 in anhydrous THF (1 mL) over 10 min. The reaction mixture was warm to 23° C. over 1.5 h then warmed to 33° C. for 2.5 h to give 94.4% conversion to compound B-4B3. The reaction was quenched with 200 μL of saturated NH₄Cl then charged with 5 mL dichloromethane and 5 mL of 1 N HCl. The phases were separated and the organic phase extracted with 2×5 mL portions 1 N HCl and 5 mL of 5% NaHCO₃. The organic phase was dried over Na₂SO₄ and filtered. The resulting residue was purified by silica gel flash chromatography using 80/20 n-heptane/acetone to give B-4B3 as a white solid (72.5 mg, 41.1%, dr=13.7:1).

### Example 2C

### Preparation of (S)-isopropyl 2-(((S)-phenoxy(pyridin-2-ylthio)phosphorothioyl)amino)propanoate (Compound 12)

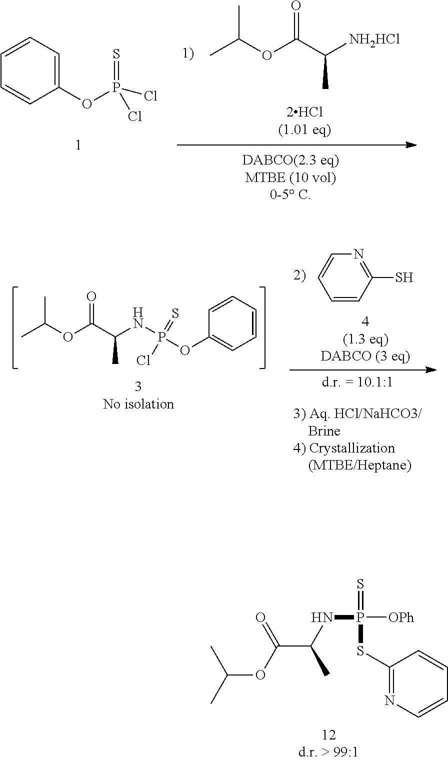

To a mixture of (S)-isopropyl 2-aminopropanoate hydrochloride (2) (149 g, 0.89 mol) and MTBE (1 L) at 0-5° C. was charged O-phenylphosphorodichlororidothioate (1) (200 g, 0.88 mol) and then slowly a mixture of DABCO (227 g, 2.02 mol) in MTBE (1.6 L). The resulting mixture was aged for ˜5 hrs in the cold and then slowly via cannula added to another vessel with DABCO (296 g, 2.64 mol) and mercaptopyridine (127 g, 1.14 mol) under nitrogen atmosphere. The mixture was aged under agitation for 1 hr in the cold, slowly warmed up to 40° C. and aged there until completion.

The mixture was then cooled to 0˜5° C. and treated with 1N HCl (0.8 L). The layers were separated and the organic solution was washed consecutively with 1N aq. HCl solution (0.8 L), 7% aq. NaHCO₃ solution (1 L) and 5% aq. NaCl solution (1 L) and then filtered through a pad of Celite. This solution was concentrated to 400 mL under reduced pressure and the temperature adjusted to 35-40° C. Heptane (260 mL) was added slowly followed by solid compound 12 (˜200 mg) as seed. The resulting slurry was cooled slowly to −0° C. and aged under agitation for at least 3 hrs. The solid was removed by filtration, washed with heptane (200 mL) and dried under vacuum at ambient temperature to provide 236 g (67.8%) of compound 12 as an off-white crystalline solid.

### Example 3A

### Diastereoselective Preparation of (2S)-isopropyl 2-(((((2R,3R,4R,5R)-5-(2,4-dioxo-3,4-dihydropyrimidin-1(2H)-yl)-3,4-dihydroxy-4-methyltetrahydrofuran-2-yl)methoxy)(phenoxy)phosphorothioyl)amino) propanoate (Compound Va)

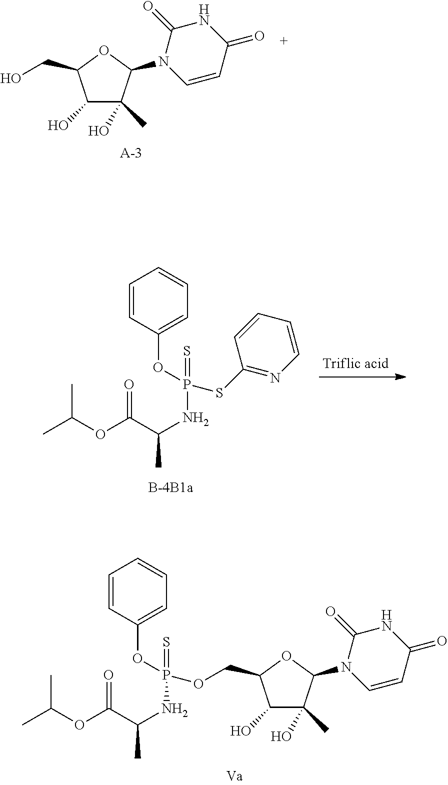

To a 50 L glass reactor equipped with a mechanical stirrer, reflux condenser, nitrogen inlet, temperature controller, and thermocouple coupled with reaction monitoring software, was charged compound A-3 (595 g, 1 eq.). A solution of compound B-4B1a (1.7 kg, 1.28 eq.) in dichloromethane (5.95 L) was prepared and added to the reaction vessel. The reaction was cooled to −2.9° C. To it, trifluoromethanesulfonic acid (548 mL, 932 g, 2.7 eq.) was added over 1 hr while maintaining the internal temperature <5° C. The internal temperature was adjusted to 0° C. and stirred for 2 hrs. Then the internal temperature was raised to 5° C. and, the mixture was stirred for another 5 hrs. The reaction temperature was increased further to 10° C. over 10 hrs and, the mixture was stirred for 2 hrs. HPLC analysis of the reaction mixture showed >96.5% conversion of compound B-4B1a. The reaction mixture was cooled to 0° C. To it, water (5.95 L) was added over 42 minutes while maintaining the internal temperature below 10° C. Then, the reaction temperature was adjusted to 10° C., and the lower organic phase was removed to a flask. The pH of the aqueous layer was adjusted to 7.0 by adding 30 wt % ammonium hydroxide (32 mL). The aqueous phase was stirred two times with IPAc (5 and 4 L, respectively). The combined organic layer was concentrated to a final volume of 3-4 L and charged to the glass reactor. To it 5.75 L of IPAc was added, and the mixture was washed three times with 2N hydrochloric acid (3×5.95 L), followed by washing with 5 wt % aqueous sodium carbonate solution (5 L). The organic layer was concentrated, using rotary evaporator at 33° C., to 6.75 L. The temperature was brought to room temperature and seeded with compound V, and the mixture was rotated for 30 minutes to ensure a slow crystallization.

The solution was heated to 34° C. and concentrated under vacuum to a final volume of 4.25 L. The solution was transferred from rotary evaporator to a clean 50 L glass reactor. The solution was further concentrated to 3 L. To it, toluene (5.95 L) was added over 1.5 hr at 50-52° C. The batch was heated to 60° C. and held at that temperature for 45 minutes. Then the temperature was lowered to 10° C. over 5 hrs and stirred at 10° C. for 12 to 63 hrs. The solid was filtered, washed with a solvent mixture toluene/MTBE (80:20, 2×2.9 L), and dried at 45-50° C. under vacuum with nitrogen sweep for 28 hrs to provide 834 g of white solid, compound Va.

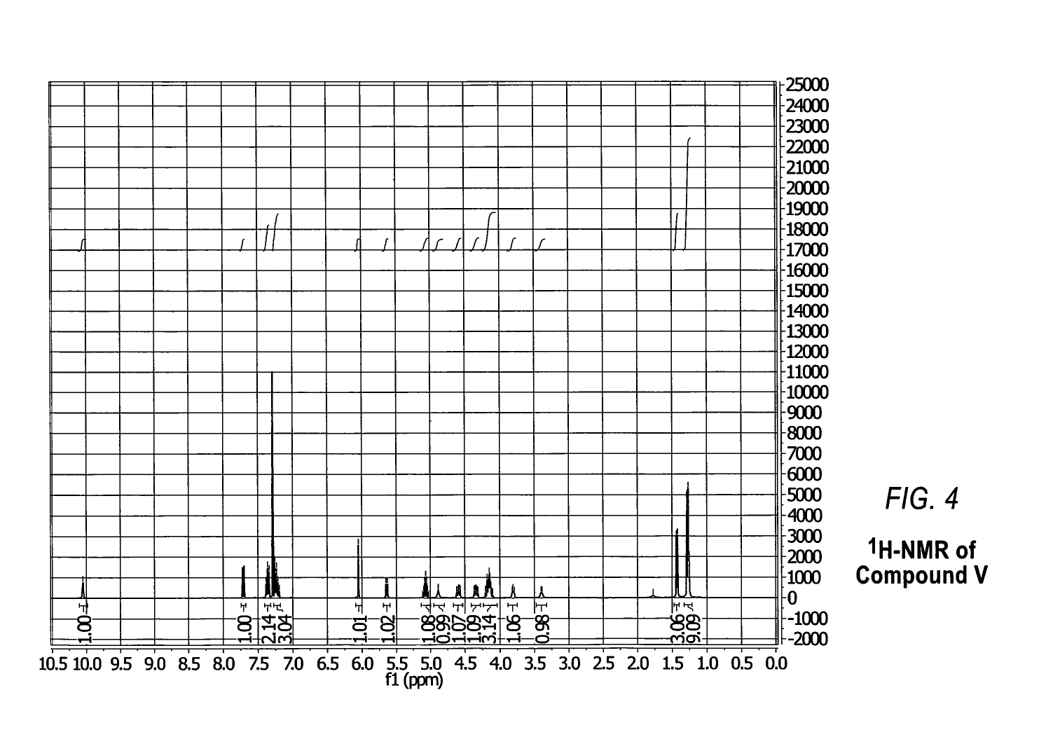

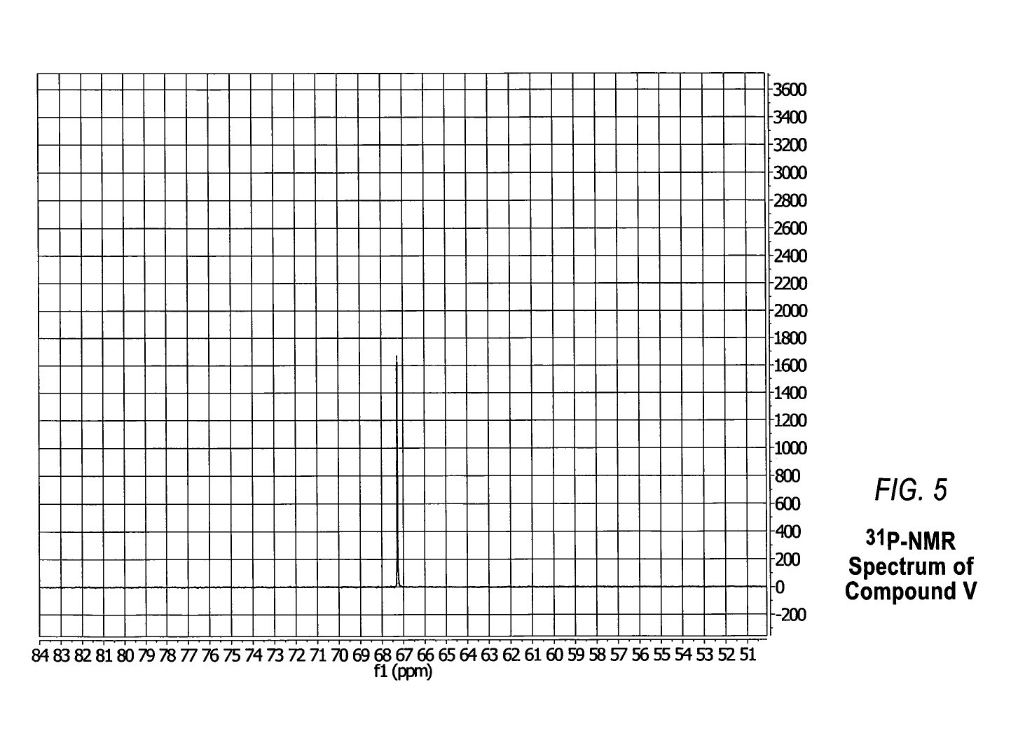

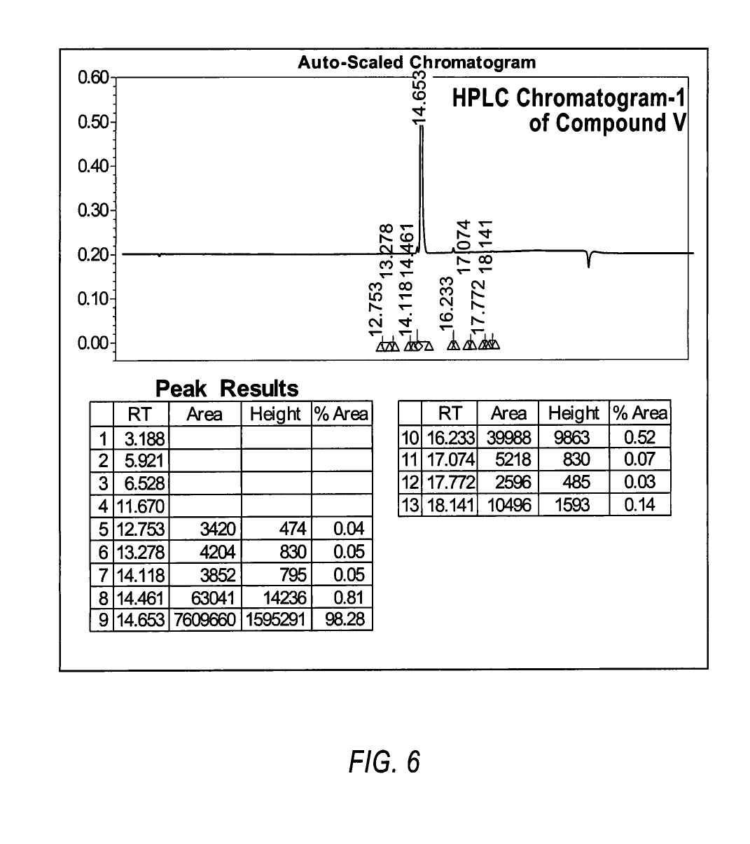

FIG. 4 is a ¹H NMR spectrum of the product (compound Va). FIG. 5 is a ³¹P NMR spectrum of the product (compound Va). A HPLC chromatogram of the reaction is provided in FIG. 6. HPLC analysis of the compound Va resulting from the above reaction was found to be at least >98%.

### Example 3B

### Diastereoselective Preparation of (2S)-isopropyl 2-(((((2R,3R,4R,5R)-5-(2,4-dioxo-3,4-dihydropyrimidin-1(2H)-yl)-3,4-dihydroxy-4-methyltetrahydrofuran-2-yl)methoxy)(phenoxy)phosphorothioyl)amino) propanoate (Compound V-1)

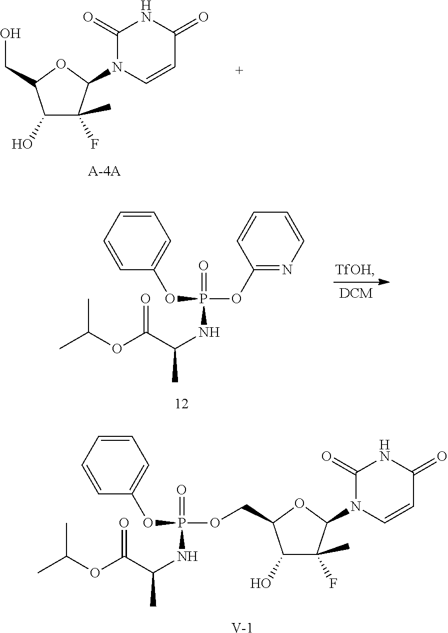

Phosphoramidate 12 (32 mg, 0.088 mmol, 1.15 equiv, dr=13.7:1) and nucleoside A-4A (20 mg, 0.077 mmol, 1.0 equiv) were charged to a vial. The vial was purged with nitrogen, charged with dichloromethane (0.3 mL) and cooled on an ice bath. Triflic acid (16.4 μL, 0.186 mmol, 2.42 equiv) was charged to the mixture over 2 min. The mixture was stirred at 0° C. for 1.5 h to give 40.7% conversion to compound V-1, which had a dr=11.6:1.

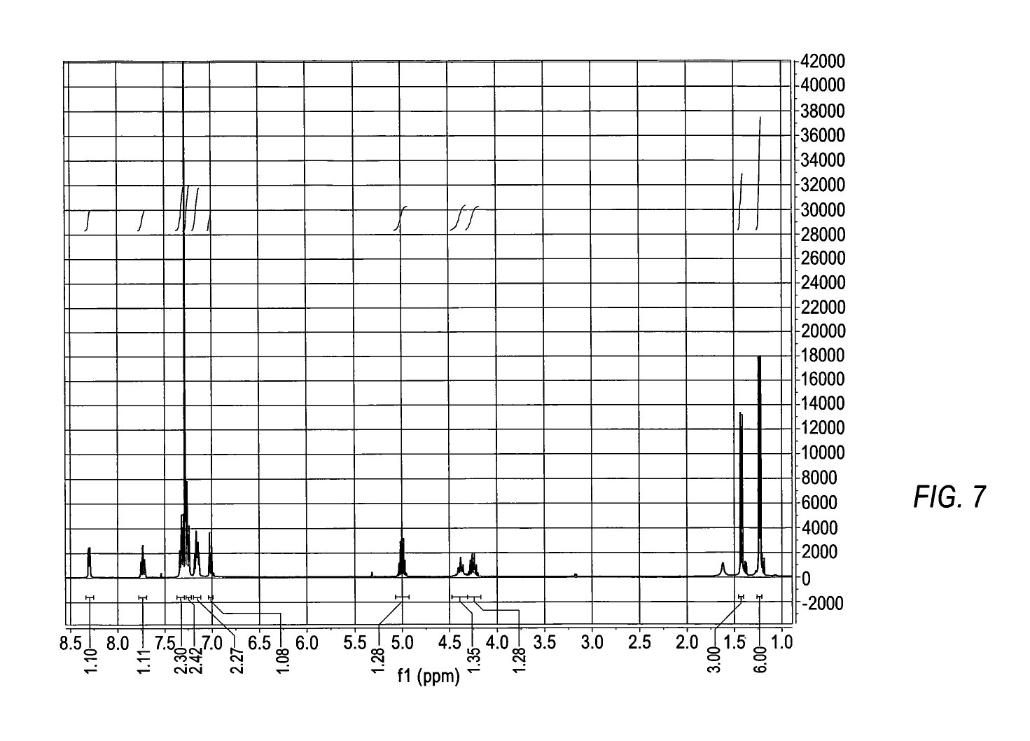

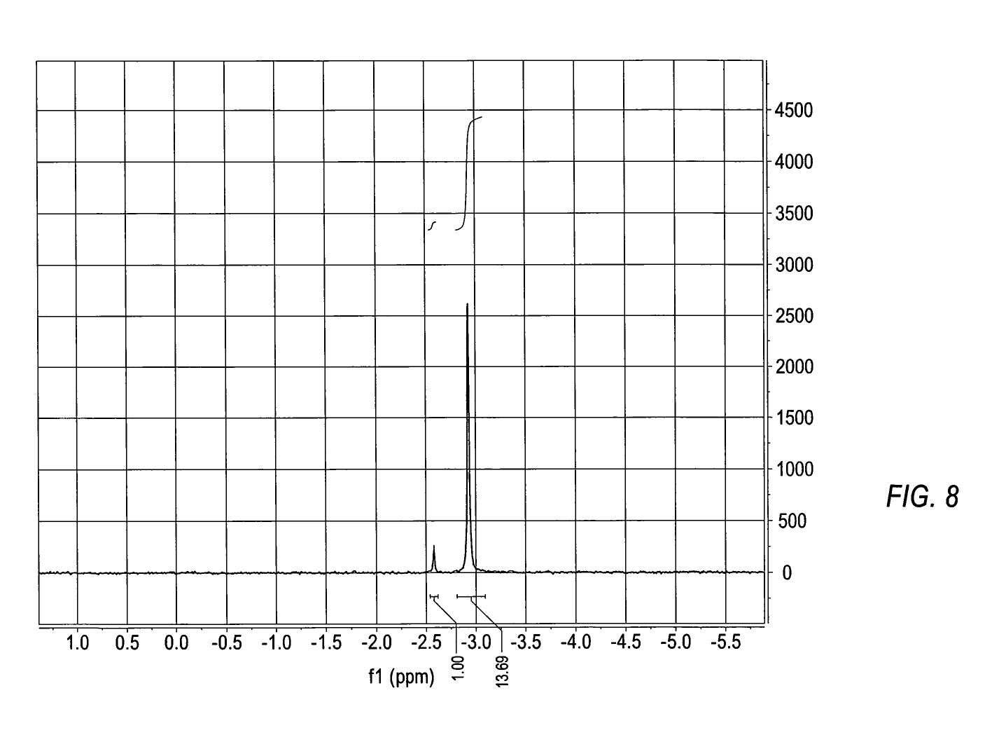

FIG. 7 is a ¹H NMR spectrum of the product (compound V-1). FIG. 8 is a³¹P NMR spectrum of the product (compound V-1).

### Example 4A

### Diastereoselective Preparation of (2S)-isopropyl 2-(((((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate (Compound V-2)

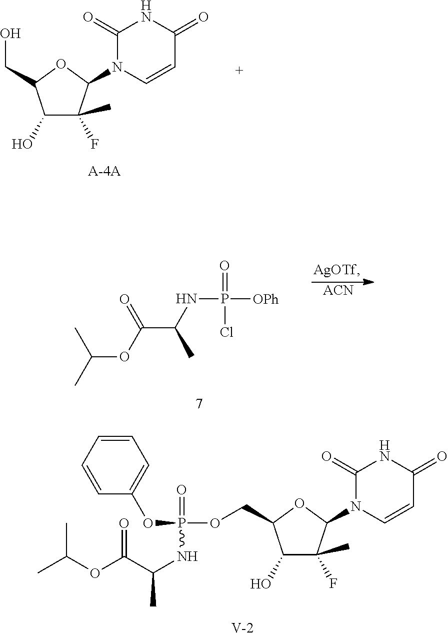

Chlorophosphoramidate 7 (441 mg, 1.44 mmol, 1.5 equiv) and nucleoside A-4A (250 mg, 0.96 mmol, 1.0 equiv) were charged to a vial followed by acetonitrile (3 mL). The vial was purged with nitrogen, cooled on an ice bath and charged with AgOTf (272 mg, 1.06 mmol, 1.1 equiv). The mixture was stirred at 0° C. for 3 h, then warmed to 23° C. and stirred for an additional 2 h to give 98.9% conversion to compound V-2 by HPLC. The mixture was cooled on an ice bath and quenched with 2 mL water. The suspension was warmed to room temperature, filtered, and the resulting solution was extracted with 5 mL dichloromethane. The extract was dried over Na₂SO₄, filtered, concentrated, and purified by silica gel flash chromatography using a 2-5% methanol in dichloromethane gradient to give a diastereomeric mixture of V-2 (283 mg, 56%).

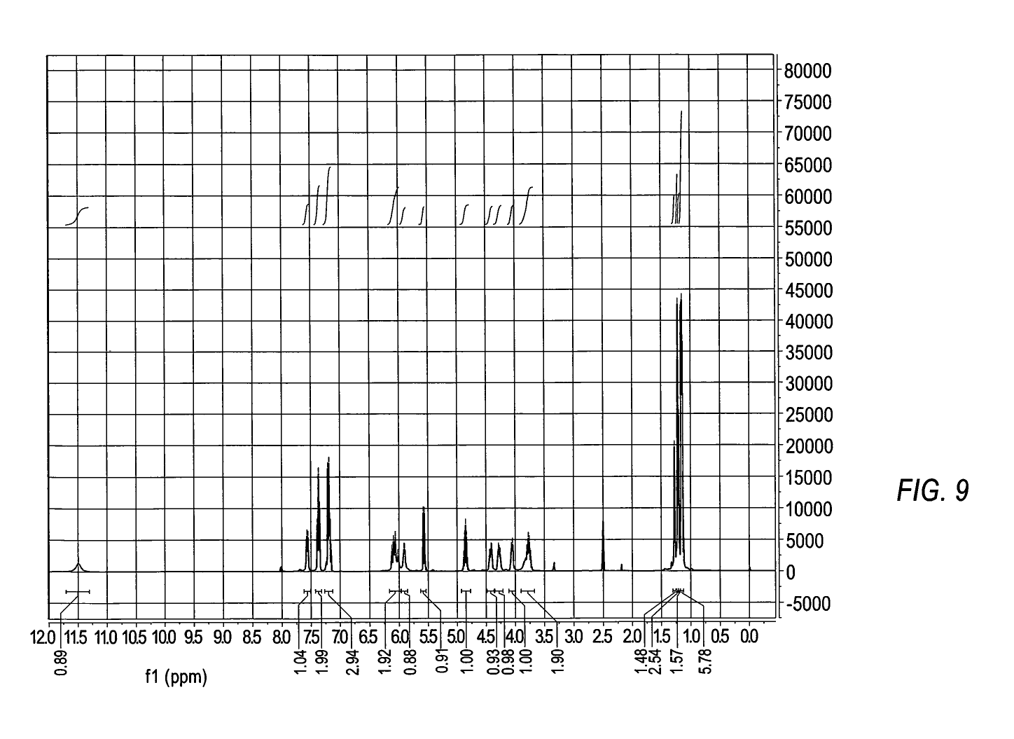

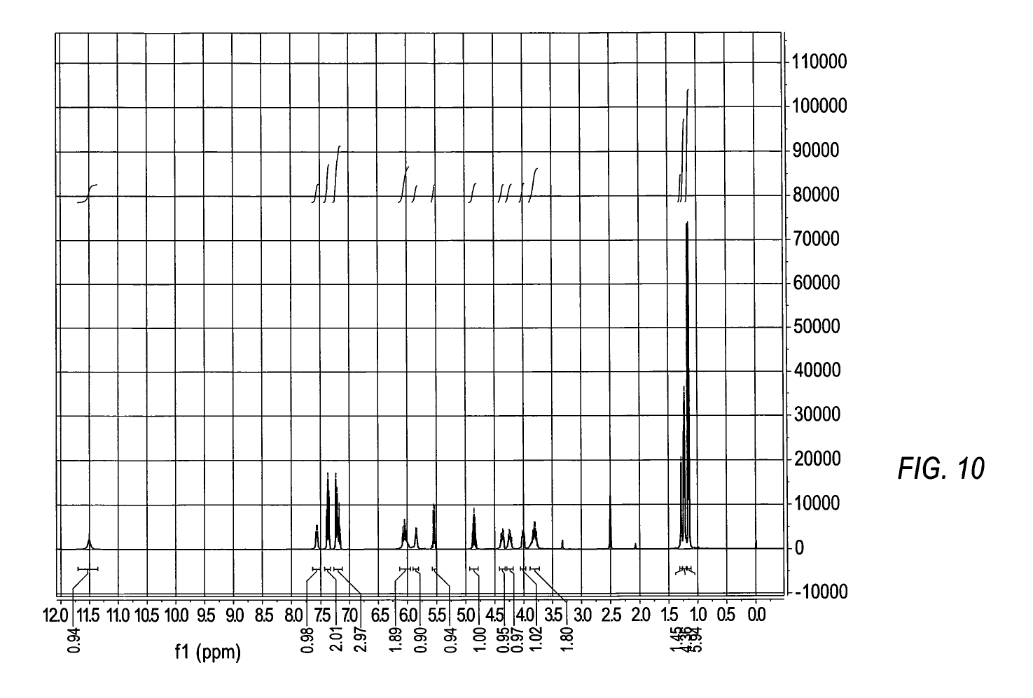

FIG. 9 is a ¹H NMR spectrum of nucleoside A-4A. FIG. 10 is a ¹H NMR spectrum of chlorophosphoramidate 7.

### Example 4B

### Diastereoselective preparation of (2S)-isopropyl 2-(((((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate (Compound V-2)

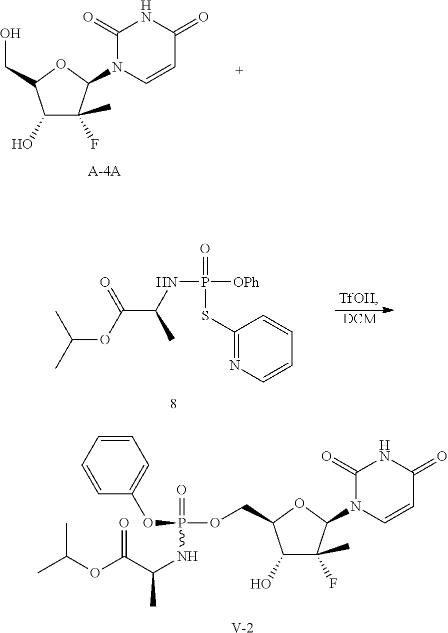

Nucleoside A-4A (100 mg, 0.0.38 mmol, 1.0 equiv) and compound 8 (175 mg, crude) were charged to a vial followed by dichloromethane (1 mL). The mixture was cooled on an ice bath and charged with triflic acid (86 μL, 2.53 equiv) over 5 min. The mixture was stirred at 0° C. for 3 h, then warmed to 23° C. and stirred for an additional 3.5 h to give 36.5% conversion to compound V-2 by HPLC.

### Example 4C-1

### Diastereoselective Preparation of (S)-isopropyl 2-(((S)-(((3aR,4R,6R,6aR)-6-(2,4-dioxo-3,4-dihydropyrimidin-1(2H)-yl)-2,2,6a-trimethyltetrahydrofuro[3,4-d][1,3]dioxol-4-yl)methoxy)(phenoxy)phosphorothioyl)amino)propanoate (Compound V-3)

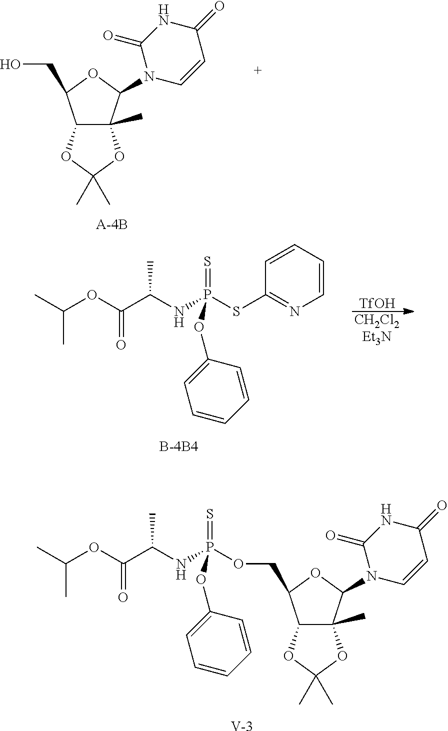

A mixture of acetonide A-4B (10 g, 33.5 mmol) and compound B-4B4 (14.5 g, 36.7 mmol) in dichloromethane (100 mL) was cooled to 10° C. At this temperature triflic acid was added (12.1 g, 80.8 mmol) slowly over a period of ¾ hrs. After the addition, the reaction mixture was aged in the cold for about 5 hrs and then slowly warmed up to 25° C. and stirred for about 15 hrs. The mixture was then cooled to 10° C. and neutralized by the slow addition of triethylamine (8.1 g, 81 mmol). To it isopropyl acetate (100 ml) and 2M aq. sodium carbonate (100 mL) were added. The layers were separated and the organic layer was washed again with 2M aq. sodium carbonate. The organic solution was then dried over sodium sulfate and concentrated to render 19.5 g of crude V-3.

### Example 4C-2

### Acetonide Deprotection of Compound V-3 to Generate Compound Va

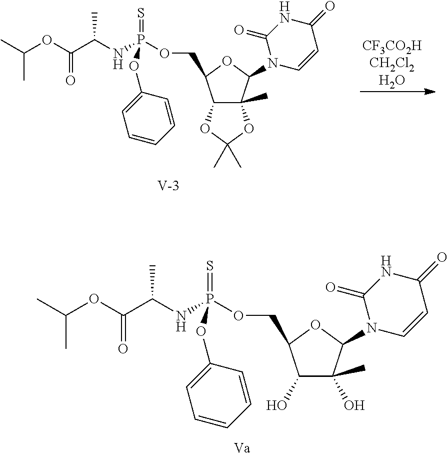

To a cooled (˜10° C.) solution of acetonide V-3 (19.5 g) in dichloromethane (78 mL), a solution of trifluoroacetic acid (98 mL) in water (19.6 mL) was added by drop-wise using an addition funnel. The cooled reaction mixture was aged under agitation (˜6 hrs) and then water (140 mL) was added slowly. The resulting solution was warmed to ambient temperature and the layers were separated. The aq. layer was extracted with dichloromethane (2×100 mL). The combined organic solutions cooled to 6-7° C. and pH was adjusted to ˜8.5 with conc. ammonium hydroxide (13 mL). The resulting mixture was concentrated and then isopropyl acetate (200 mL) was added, and the mixture was concentrated again. This process was repeated one more time. The residue was dissolved in isopropyl acetate (300 mL) and washed with 1.5M aq. sodium carbonate (100 mL). The layers were separated and the organic solution was concentrated to dryness under reduced pressure. Isopropyl acetate (80 mL) was added to the residue and the resulting mixture was heated to 60° C. At this temperature toluene (100 mL) was added slowly and the resulting slurry was slowly cooled to 10° C. and aged for ˜15 hrs. The solid was collected by filtration and washed with 9:1 toluene/IPAc (60 mL). After drying, 15.5 g of compound V was obtained over two steps.

### Example 5

### Alternative Reaction Conditions

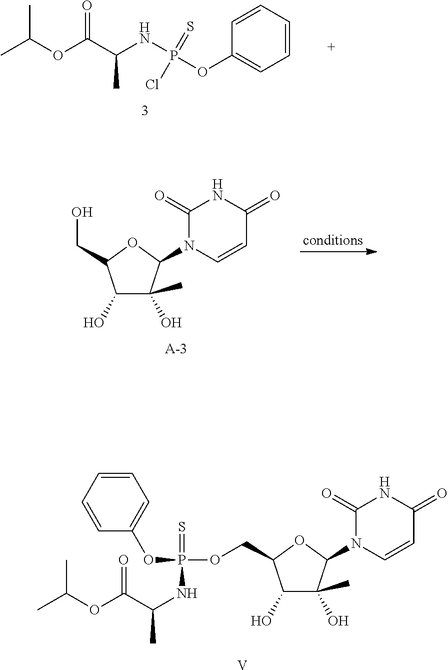

The chemical reactions for coupling compound 3 with compound A-3 were performed using procedures discussed in Examples, 1-3, above and the reaction conditions specified in Table 1, below.

TABLE 1
Reaction conditions.

| | | | Conversion | |
|---|---|---|---|---|
| Exp. | Conditions | Solvent | of A-3¹ | d.r. |
| 5a | AgOTf (1.1 equiv), 23° C. | ACN | 96.5% | 0.95:1 |
| 5b | TfOH (1.1 equiv), 23° C. | ACN | 89.1% | 0.94:1 |
| 5c | KOTf (2.0 equiv), 35° C. | ACN | 90.8% | 0.94:1 |

¹Conversion was calculated from HPLC data by the following: (100 × (AUC compound A-3/(AUC compound V + AUC compound 3)).

AgOTf, TfOH, and triflate salts were shown to provide the compound of Formula V stereospecifically from a single diastereomer of the compound 3.

### Example 6

### Triflic Acid Equivalents

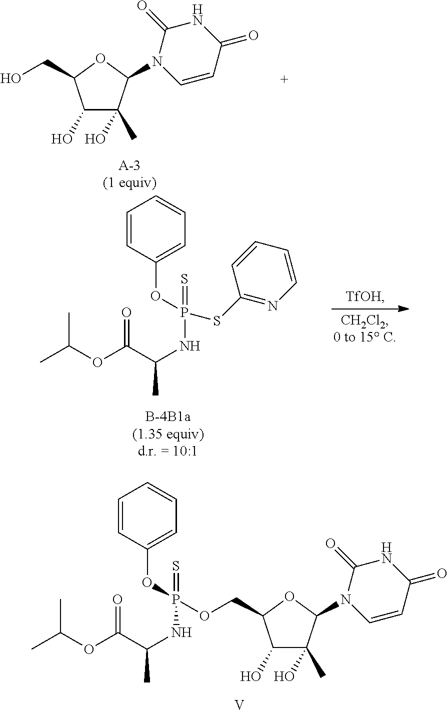

Chemical reactions for coupling compound A-3 with compound B-4B1a were performed using procedures discussed in Examples 1-3 and the reaction conditions specified below in Table 2.

TABLE 2
Triflic acid equivalents.

| | Equiv | Conversion of | Amount B-4B1a |
|---|---|---|---|
| Example | TfOH² | A-3³ | remaining |
| 6a | 2.00 | 96.2% | 2.3% |
| 6b | 1.50 | 70.1% | 6.8% |
| 6c | 1.25 | 67.0% | 7.2% |
| 6d | 0.90 | 1.8% | 25.4% |

²Equivalents are based on the amount of compound B-4B1a used (1.35 equiv).
³Conversion was calculated from HPLC data by the following: (100 × (AUC compound A-3/(AUC compound V + AUC compound B-4B1a)).

### Example 6A

### Equivalents of Compound B-4B1a

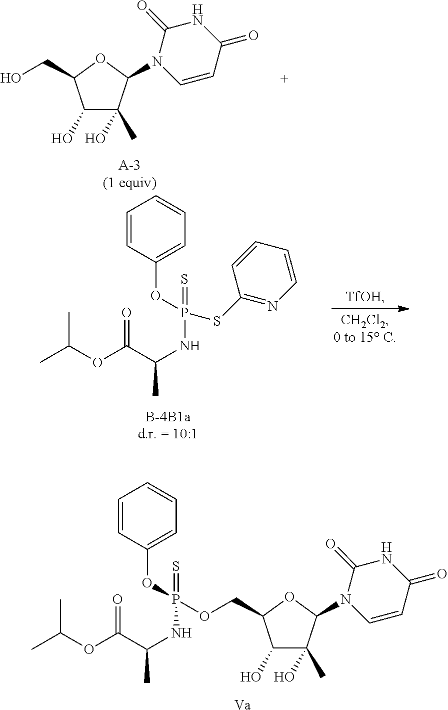

Chemical reactions for coupling compound A-3 with compound B-4B1a were performed using procedures discussed in Examples 1-3 and the reaction conditions specified below in Table 3.

TABLE 3
Compound B-4B1a equivalents.

| | Equiv. Compound | Conversion | Yield |
|---|---|---|---|
| Example | B-4B1a | of A-3³ | (Compound Va) |
| 6-1 | 1.35 | 96.7% | 78% |
| 6-2 | 1.275 | 93.5% | 76% |
| 6-3 | 1.20 | 81.9% | 70% |

### Example 6B

### Equivalents of Compound B-4B1a

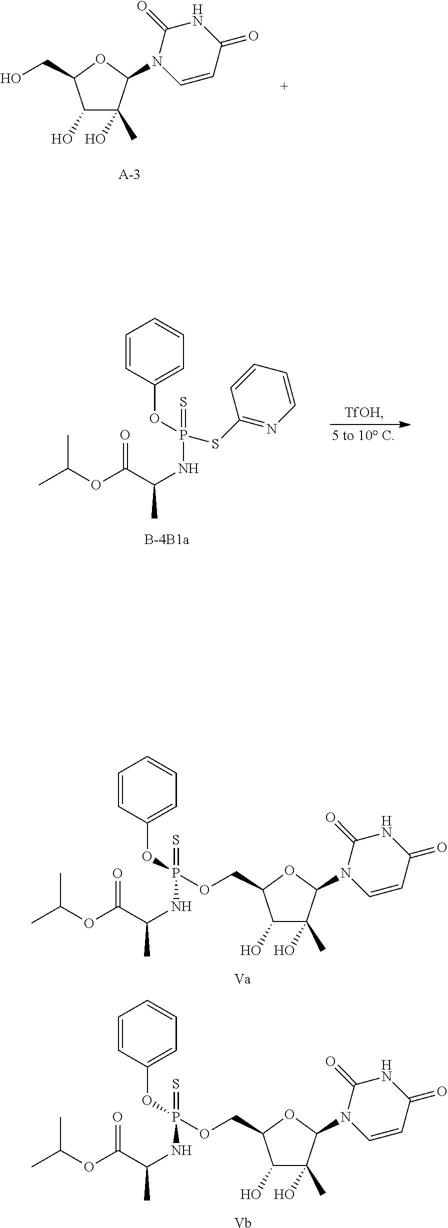

Chemical reactions for coupling compound A-3 with compound B-4B1a were performed using procedures discussed in Examples 1-3 and the reaction conditions specified below in Table 4.

TABLE 4
Compound B-4B1a equivalents.

| | Scale | Equiv. | | d.r. | |
|---|---|---|---|---|---|
| | (g of | of | Conversion | (Cmpd Va:Cmpd | Yield |
| Example | A-3) | B-4B1a | of A-3³ | Vb) | (Va + Vb) |
| 6-4 | 3 g | 1.20 | 89% | 95.1:4.9 | 79% |
| 6-5 | 3 g | 1.30 | 96% | 96.1:3.9 | 75% |
| 6-6 | 10 g | 1.30 | 94% | 96.1:3.9 | — |
| 6-7 | 8 g | 1.30 | 92% | 96.7:3.3 | — |
| 6-8 | 3 g | 1.35 | 97% | 95.6:4.4 | 80% |
| 6-9 | 3 g | 1.40 | 97% | 98.0:2.0 | — |
| 6-10 | 3 g | 1.45 | 96% | 98.5:1.5 | 79% |

### Example 7A

### Solvents Useful for Generating Compound Va

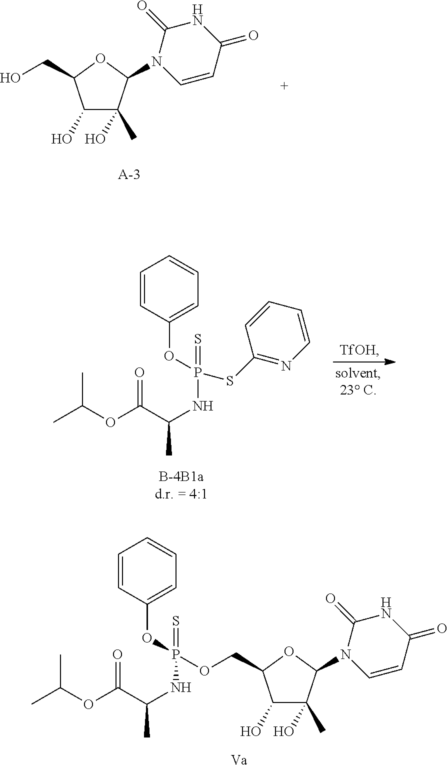

Chemical reactions for coupling compound A-3 with compound B-4B1a were performed using procedures discussed in Examples 1-3 and the reaction conditions specified below in Table 5.

TABLE 5
Solvents.

| Solvent | Temp | Conversion of A-3³ | d.r. |
|---|---|---|---|
| 1,4-dioxane | 23° C. | 97% | 3.6:1 |
| cyclopentylmethyl ether | 23° C. | 58% | 3.8:1 |
| dichloromethane | 23° C. | 97% | 40.7:1 |
| chloroform | 23° C. | 96% | 29.1:1 |
| trifluorotoluene | 23° C. | 90% | 13.7:1 |
| toluene | 23° C. | 91% | 11.6:1 |
| 1,2-dichlorobenzene | 23° C. | 86% | 12.7:1 |
| fluorobenzene | 23° C. | 93% | 22.7:1 |

### Example 7B

### Solvents Useful for Generating Compound Va

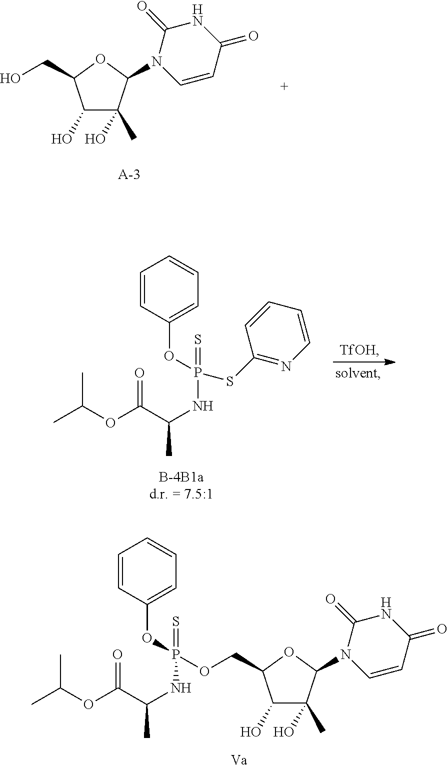

Chemical reactions for coupling compounds of Formula A-3 with compounds of Formula B-4B1a were performed using procedures discussed in Examples 1-3 and the reaction conditions specified below in Table 6.

TABLE 6
Solvents and reaction conditions.

| | | | Con- | | Yield |
|---|---|---|---|---|---|
| Exam- | | Con- | version | | (Compound |
| ple | Solvent | ditions | of A-3³ | d.r. | Va) |
| 7-1 | 1,4-dioxane | 23° C. | 91.2% | 84.3:15.6 | 74% |
| 7-2 | dichloromethane | 0 to | 93.7% | 94.3:5.7 | 84% |
| | | 18° C. | | | |

### Example 8

### Thiophosphorylation in Difference Solvents

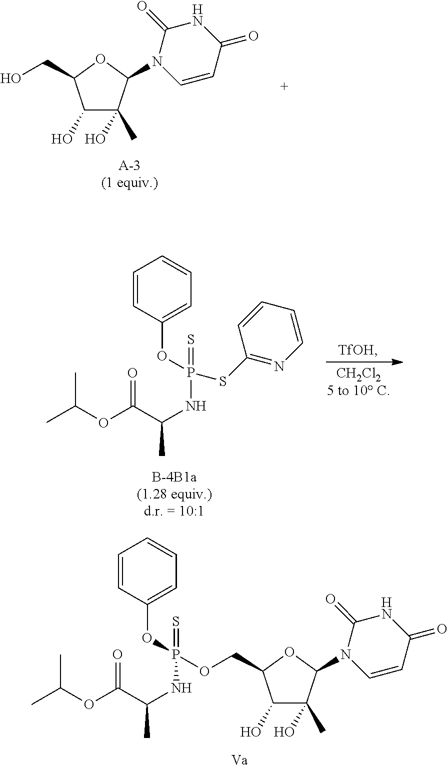

Chemical reactions for coupling compound A-3 with compound B-4B1a were performed using procedures discussed in Examples 1-3 and the reaction conditions specified below in Table 7.

TABLE 7
Solvent mixtures.

| | Dissolved Slurry | Filtered Solid | Conversion |
|---|---|---|---|
| | (Cmpd Va:Cmpd | (Cmpd Va:Cmpd | of |
| Reaction Solvent | Vb) | Vb) | A-3³ |
| DCM/toluene (5:1) | 95.7:4.3 | 99.4:0.6 | ~96% |
| DCM/dioxane (4:1) | 92.9:7.1 | — | 78.8% |
| DCM/dioxane (1:1) | 92.6:7.4 | — | 81.6% |
| DCM/sulfolane | 90.5:9.5 | — | 87.4% |
| (1:1) | | | |

### Example 9A

### Stereoselective Activators for Generating Compound B-4B1

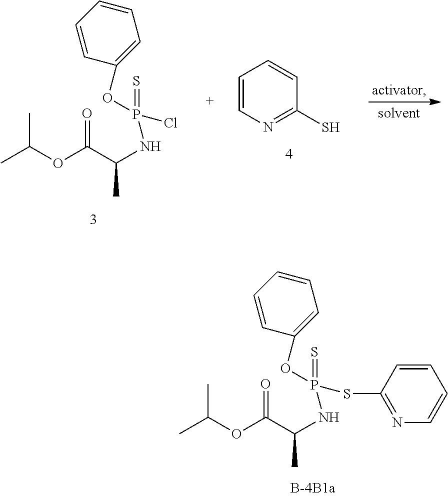 [SMILES: CC(C)OC(=O)[C@H](C)NP(=S)(Cl)Oc1ccccc1 ; Sc1ccccn1 ; CC(C)OC(=O)[C@H](C)NP(=S)(Oc1ccccc1)Sc1ccccn1]

Compound B-4B1a was generated according to the procedures discussed in Examples 1-3 and the reaction conditions specified below in Table 8.

TABLE 8
Reaction conditions.

| Activator | Solvent | Temperature | Conversion³ | d.r. |
|---|---|---|---|---|
| Et₃N | DCM | 23° C. | 100% | 1:1 |
| NMI | THF | 0 to 23° C. | 100% | 4.2:1 |
| DMAP | THF | 0 to 23° C. | 100% | 4.1:1 |
| 3,4-lutidine | THF | 0 to 23° C. | 20% | 2.1:1 |
| 4-methoxypyridine | THF | 0 to 23° C. | 45% | 2.6:1 |
| Imidazole | THF | 0 to 23° C. | 0% | — |
| N-methylpyrrolidine | THF | 0 to 23° C. | 63% | 1.5:1 |
| DABCO | THF | 0 to 23° C. | 100% | 7.1:1 |

### Example 9B

### Solvents for Stereoselective Activators

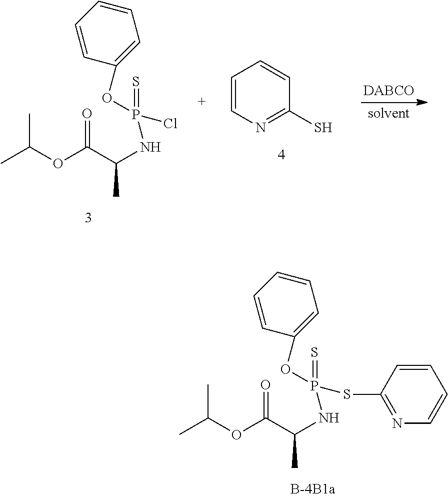 [SMILES: CC(C)OC(=O)[C@H](C)NP(=S)(Cl)Oc1ccccc1 ; Sc1ccccn1 ; CC(C)OC(=O)[C@H](C)NP(=S)(Oc1ccccc1)Sc1ccccn1]

Compound B-4B1a was generated according to the procedures discussed in Examples 1-3 and the reaction conditions specified below in Table 9.

TABLE 9
Solvents.

| Solvent | Temperature | d.r. |
|---|---|---|
| THF | 0 to 23° C. | 88:12 |
| DCM | 0 to 23° C. | 71:29 |
| ACN | 0 to 23° C. | 63:37 |
| Toluene | 0 to 23° C. | 89:11 |
| MTBE | 0 to 23° C. | 92:8 |
| Butanone | 0 to 23° C. | 81:19 |
| cyclopentylmethyl ether | 0 to 23° C. | 91:9 |
| EtOAc | 0 to 23° C. | 90:10 |
| tBuOAc | 0 to 23° C. | 89:11 |
| IPAc | 0 to 23° C. | 89:11 |
| MIBK | 0 to 23° C. | 85:15 |
| MeTHF | 0 to 23° C. | 89:11 |
| MTBE/heptane | 0 to 23° C. | 92:8 |
| (75:25) | | |

### Example 10

### Stereoselectivity and Nucleophiles

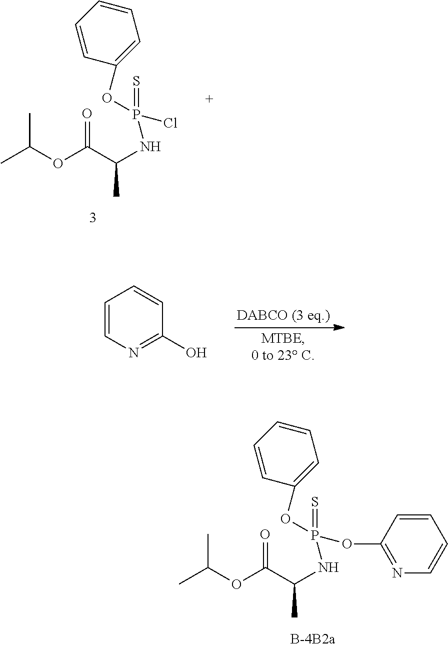 [SMILES: CC(C)OC(=O)[C@H](C)NP(=S)(Cl)Oc1ccccc1 ; Oc1ccccn1 ; CC(C)OC(=O)[C@H](C)NP(=S)(Oc1ccccc1)Oc1ccccn1]

Compound B-4B2a was generated according to the procedures discussed in Examples 1-3 and the reaction conditions specified below in Table 10.

TABLE 10
Stereoselectivity of select nucleophiles.

| | | Yield | |
|---|---|---|---|
| Nucleophile | Temperature | (Compound B-4B2a) | d.r. |
| 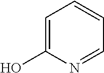 | 0 to 23° C. | 61.8% | 92:8 |
| 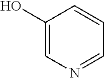 | 0 to 23° C. | >98% | 67:33 |
| 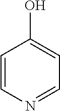 | 0 to 23° C. | 70.6% | 60:40 |
| 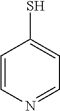 | 0 to 23° C. | 60.8% | 66:34 |
| 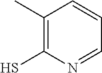 | 0 to 23° C. | >98% | 87:13 |
| 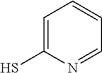 | 0 to 23° C. | >98% | 92:8 |

### OTHER EMBODIMENTS

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the examples disclosed herein are illustrative only and are not intended to limit the scope of the present disclosure, but rather to also cover all modification and alternatives coming within the true scope and spirit of the invention.

1. A method of preparing a compound of Formula I: 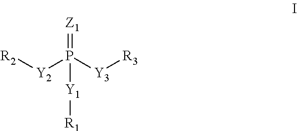
or pharmaceutically acceptable salt thereof wherein:
Z₁ is O or S;
Each of Y₁, Y₂ and Y₃ is independently a bond, —S—, —O—, or —NR₁₀₀—,
R₁₀₀ is hydrogen, C₁₋₆ alkyl, C₂₋₆ alkenyl, C₂₋₆ alkynyl, aryl, heteroaryl, aryl(C₁₋₆ alkyl), C₃₋₈ cycloaliphatic, or a saturated, partially unsaturated, or fully unsaturated 3-8 membered heterocyclic ring having up to 3 heteroatoms independently selected from N, O, or S; and
Each of R₁, R₂ and R₃ is independently -L-R₅, wherein
Each L is independently a bond, —(CH₂)ₘ—, —(CH₂)ₘ(CHR₆)ₚ—, —(CH₂)ₘ—(CR₆R₇)ₚ—, or —(C(R₈)₂)ₘC(O)O—,
Each of R₆ and R₇ is independently selected from hydrogen, halogen, —OH, —N(R₈)₂, or −OR₈,
Each R₈ is independently hydrogen or C₁₋₆ alkyl,
Each m is independently 0-3,
Each p is independently 0-3,
Each R₅ is independently hydrogen, —O⁻, —OH, alkoxy, C₁₋₆ alkyl, C₂₋₆ alkenyl, C₂₋₆ alkynyl, —(C(R₈)₂)ₘC(O)OR₈, aryl, aryl(C₁₋₆ alkyl), C₃₋₈ cycloaliphatic, heteroaryl, a saturated or partially unsaturated 3-8 membered heterocyclic ring having up to 3 heteroatoms independently selected from N, O, or S, an optionally substituted amine, an optionally substituted N-linked amino acid, an optionally substituted N-amino acid ester derivative, or 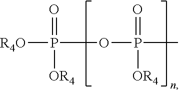
wherein each R₄ is independently absent or hydrogen, and n is 0 or 1, and wherein the alkyl, alkenyl, alkynyl, aryl, aryl-(C₁₋₆ alkyl), cycloaliphatic, heteroaryl, or heterocyclic ring groups are each optionally substituted with 1-3 groups independently selected from halo, —OH, —CN, azido, optionally substituted C₁₋₆ alkyl, optionally substituted C₁₋₆ alkoxy, an optionally substituted heterocyclic base, or an optionally substituted heterocyclic base with a protected amino group;
comprising the step of:
i) reacting a compound of Formula A with a compound of Formula B 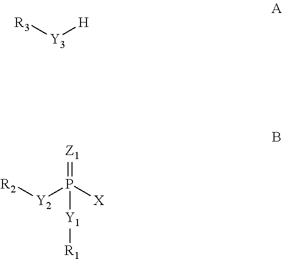
wherein X is a leaving group, in the presence of an acid or a metal salt, to generate the compound of Formula I.

2. The method of claim 1, wherein
Y₁ is a bond;
Y₂ and Y₃ are each independently —O—, or —S—;
R₁ is —O⁻, —OH, alkoxy, an optionally substituted amine, an optionally substituted N-linked amino acid or an optionally substituted N-amino acid ester derivative; and
Each of R₂ and R₃ is independently hydrogen, C₁₋₆ alkyl, aryl, heteroaryl, aryl(C₁₋₆ alkyl), or C₃₋₈ cycloaliphatic.

3. The method of claim 1, wherein R₁₀₀ is hydrogen or C₁₋₆ alkyl.

4. The method of claim 1, wherein —Y₁—R₁ is an optionally substituted N-linked amino acid or an optionally substituted N-amino acid ester derivative; and R₂ is an optionally substituted aryl.

5. The method of claim 4, wherein —Y₁—R₁ is 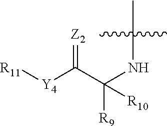
wherein
Z₂ is O or S;
Y₄ is a bond, —S—, —O—, or —NR₁₀₀—;
Each of R₉ and R₁₀ is independently selected from hydrogen, C₁₋₆ alkyl, halo-C₁₋₆ alkyl, C₃₋₈ cycloalkyl, aryl, aryl(C₁₋₆ alkyl), heterocyclyl, or (C₁₋₆ alkyl)heterocyclyl, or
R₉ and R₁₀ taken together with the carbon atom to which they are attached form a C₃₋₆ cycloalkyl; and
R₁₁ is hydrogen, C₁₋₆ alkyl, C₃₋₈ cycloalkyl, aryl, aryl(C₁₋₆ alkyl), or halo-C₁₋₆ alkyl.

6. The method of claim 4, wherein R₂ is an unsubstituted phenyl.

7. The method of claim 1 wherein the reaction of step i) occurs in the presence of an acid.

8. The method of claim 7, wherein the acid is a strong acid.

9. The method of claim 8, wherein the strong acid is a sulfonic acid.

10. The method of claim 9, wherein the acid is trifluoromethanesulfonic acid or methanesulfonic acid.

11. The method of claim 1, wherein the reaction of step i) occurs in the presence of a metal salt.

12. The method of claim 11, wherein the metal salt is a metal salt of trifluoromethanesulfonate, a metal salt of acetate, or a metal salt of fluoroborate.

13. The method of claim 12, wherein the metal salt is sodium trifluoromethanesulfonate, potassium trifluoromethanesulfonate, silver trifluoromethanesulfonate, indium(III) trifluoromethanesulfonate, scandium(III) trifluoromethanesulfonate, copper(II) trifluoromethanesulfonate, magnesium trifluoromethanesulfonate, palladium(II) acetate, copper(I) acetate, tetrakis(acetonitrile)copper(I) hexafluorophosphate, silver tetrafluoroborate, silver hexafluorophosphate, or any combination thereof.

14. (canceled)

15. The method of claim 1, wherein X is —W—R₁₂;
W is a bond, —S—, or —O—; and
R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl, or
—W—R₁₂ is 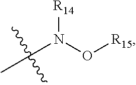
wherein each of R₁₄ and R₁₅ is independently C₁₋₆ alkyl, cycloalkyl, or heteroalkyl, or
R₁₄ and R₁₅, taken together with the heteroatoms to which they are attached, form a 6-10-membered heterocyclic ring optionally substituted with 1-3 of R₁₃.

16. The method of claim 15, wherein W is —S— or —O—; and
R₁₂ is a 5-6-membered monocyclic heteroaryl having 1-3 heteroatoms independently selected from N, O, or S, optionally substituted with 1-3 of R₁₃, wherein R₁₃ is an optionally substituted C₁₋₆ alkyl.

17. The method of claim 16, wherein —W—R₁₂ is selected from

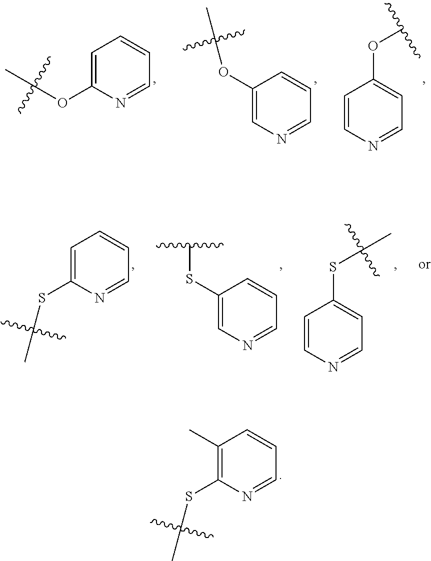

18. The method of claim 15, wherein R₁₂ is an 8-10-membered bicyclic heteroaryl having 1-4 heteroatoms independently selected from N, O, or S, optionally substituted with 1-3 of R₁₃, wherein R₁₃ is an optionally substituted C₁₋₆ alkyl.

19. The method of claim 18, wherein —W—R₁₂ is selected from

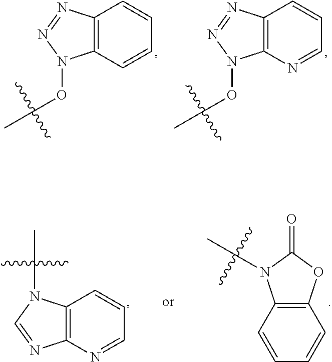 [SMILES: *On1nnc2ccccc21 ; *On1nnc2cccnc21 ; *n1cnc2ncccc21 ; *n1c(=O)oc2ccccc21]

20. The method of claim 15, wherein —W—R₁₂ is 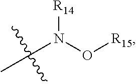
wherein R₁₄ and R₁₅ are each independently C₁₋₆ alkyl, cycloalkyl, or heteroalkyl, or R₁₄ and R₁₅ taken together with the heteroatoms to which they are attached form a 6-10 membered heterocyclic ring optionally substituted with 1-3 of R₁₃.

21. The method of claim 20, wherein —W—R₁₂ is selected from

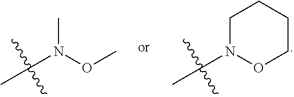

22. The method of claim 15, wherein R₁₂ is a 5-6-membered fully saturated or partially unsaturated heterocyclic ring having 1-2 heteroatoms independently selected from N, 0, or S, optionally substituted with 1-3 of R₁₃.

23. The method of claim 22, wherein —W—R₁₂ is

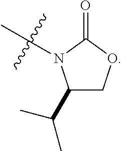 [SMILES: *N1C(=O)OC[C@H]1C(C)C]

24. The method of claim 15, wherein the compound of Formula B is a compound of Formula B-2a or B-2b:

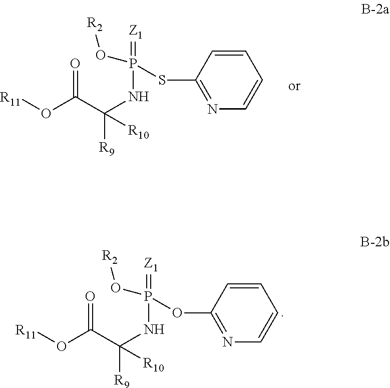

25. The method of claim 1, wherein the reaction of step i) occurs in the presence of an organic solvent.

26. The method of claim 25, wherein the organic solvent of step i) is an aprotic organic solvent.

27. The method of claim 26, wherein the aprotic organic solvent is acetonitrile, toluene, dichloromethane, 1,4-dioxane, sulfolane, cyclopentylmethyl ether, chloroform, trifluorotoluene, 1,2-dichlorobenzene, fluorobenzene, or any combination thereof.

28. The method of claim 1, wherein the reaction of step i) is performed at a temperature of about 30° C. or less.

29. (canceled)

30. The method of claim 15, further comprising the step of:
ii) reacting a compound of Formula B-3, wherein X^{A} is halogen, with H—W—R₁₂ 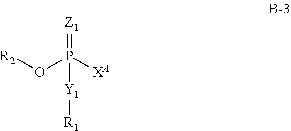
in the presence of a base to generate the compound of Formula B-1

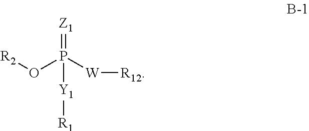

31. The method of claim 30, wherein the base in step ii) is selected from N(Et)₃, N-methylimidazole, 4-dimethylaminopyridine, 3,4-lutidine, 4-methoxypyridine, N-methylpyrrolidine, 1,4-diazabicyclo[2.2.2]octane, or any combination thereof.

32. The method of claim 30, wherein the reaction of step ii) is performed in the presence of an organic solvent.

33. The method of claim 32, wherein the organic solvent of step ii) is an aprotic organic solvent.

34. The method of claim 33, wherein the aprotic organic solvent is tetrahydrofuran, dichloromethane, acetonitrile, toluene, methyl tert-butyl ether, butanone, cyclopentylmethyl ether, ethyl acetate, tert-butyl acetate, iso-propyl acetate, methyl-iso-butyl ketone, 2-methyltetrahydrofuran, heptane, or any combination thereof.

35. The method of claim 30, wherein the reaction of step ii) is performed at a temperature of about 30° C. or less.

36. (canceled)

37. The method of claim 30, wherein the compound of Formula B-3 is a compound of Formula B-4:

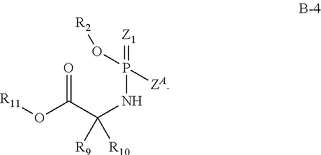

38. The method of claim 37, further comprising the step of:
iii) reacting a compound of Formula B-5, wherein X^{B} is halogen, with a compound of Formula C: 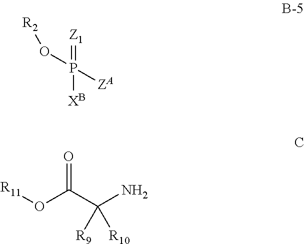
under nucleophilic substitution conditions to generate the compound of Formula B-4.

39. A method of preparing a compound of Formula II: 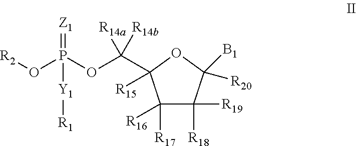
or a pharmaceutically acceptable salt thereof; wherein:
Z₁ is S or O;
B₁ is an optionally substituted heterocyclic base or an optionally substituted heterocyclic base with a protected amino group;
Y₁—R₁ is —O⁻, —OH, alkoxy, an optionally substituted amine, an optionally substituted N-linked amino acid or an optionally substituted N-amino acid ester derivative;
R₂ is an optionally substituted aryl, an optionally substituted heteroaryl, an optionally substituted heterocyclyl or 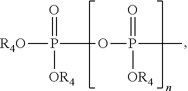
wherein each R₄ is independently absent or hydrogen, and n is 0 or 1;
Each of R₁₄ₐ and R_{14b} is independently selected from hydrogen, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or
R₁₄ₐ and R_{14b} taken together with the carbon atom to which they are attached form an optionally substituted C₃₋₆ cycloalkyl;
R₁₅ is hydrogen, azido, an optionally substituted C₁₋₆ alkyl, an optionally substituted C₂₋₆ alkenyl, or an optionally substituted C₂₋₆ alkynyl;
Each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or
R₁₇ and R₁₈ are both oxygen atoms that are linked together by —(CR₂₁R₂₂)— or by a carbonyl group;
R₂₀ is hydrogen, halogen, azido, cyano, an optionally substituted C₁₋₆ alkyl, or —OR₂₁; and
Each of R₂₁ and R₂₂ is independently selected from hydrogen, optionally substituted C₁₋₆ alkyl or optionally substituted C₃₋₆ cycloalkyl;
comprising the step of:
ia) reacting a compound of Formula A-1 with a compound of Formula B-X 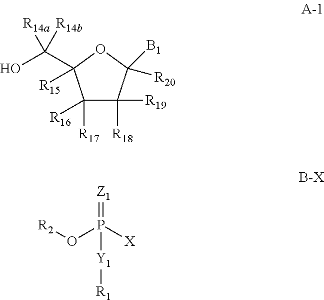
wherein X is a leaving group, in the presence of an acid or a metal salt, to generate the compound of Formula II.

40-76. (canceled)

77. A method of preparing a compound of Formula III: 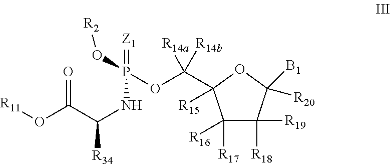
or a pharmaceutically acceptable salt thereof, having a diastereomeric purity of about 75% or greater, wherein
Z₁ is O or S;
B₁ is an optionally substituted heterocyclic base or an optionally substituted heterocyclic base with a protected amino group;
R₃₄ is C₁₋₆ alkyl, halo-C₁₋₆ alkyl, C₃₋₈ cycloalkyl, aryl, or aryl(C₁₋₆ alkyl);
R₁₁ is hydrogen, C₁₋₆ alkyl, C₃₋₈ cycloalkyl, aryl, aryl(C₁₋₆ alkyl), or halo-C₁₋₆ alkyl;
R₂ is an optionally substituted aryl, an optionally substituted heteroaryl, an optionally substituted heterocyclyl or 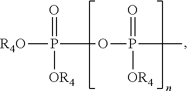
wherein each R₄ is independently absent or hydrogen, and n is 0 or 1;
Each of R₁₄ₐ and R_{14b} is independently selected from hydrogen, deuterium, an optionally substituted C₁₋₆ alkyl, an optionally substituted C₂₋₆ alkenyl, an optionally substituted C₂₋₆ alkynyl, an optionally substituted halo-C₁₋₆ alkyl, aryl, or aryl(C₁₋₆ alkyl), or
R₁₄ₐ and R_{14b} taken together with the carbon atom to which they are attached form an optionally substituted C₃₋₆ cycloalkyl;
R₁₅ is hydrogen, azido, an optionally substituted C₁₋₆ alkyl, an optionally substituted C₂₋₆ alkenyl, or an optionally substituted C₂₋₆ alkynyl;
Each of R₁₆, R₁₇, R₁₈, and R₁₉ is independently selected from hydrogen, —OH, halogen, azido, cyano, an optionally substituted C₁₋₆ alkyl, —OR₂₁ or —OC(O)R₂₂, or
R₁₇ and R₁₈ are both oxygen atoms that are linked together by —(CR₂₁R₂₂)— or by a carbonyl group;
R₂₀ is hydrogen, halogen, azido, cyano, an optionally substituted C₁₋₆ alkyl, or —OR₂₁; and
Each of R₂₁ and R₂₂ is independently selected from hydrogen, optionally substituted C₁₋₆ alkyl or optionally substituted C₃₋₆ cycloalkyl;
comprising the step of:
ib) reacting a compound of Formula A-1 and a compound of Formula B-1B 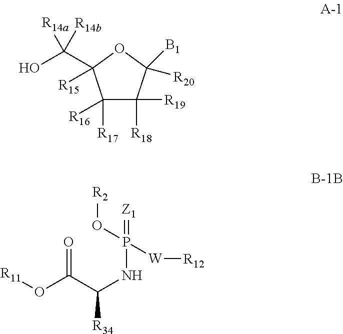
in the presence of an acid or a metal salt, wherein
W is a bond, —S—, or —O—; and
R₁₂ is a 5-10-membered mono- or bicyclic saturated, partially unsaturated, or fully unsaturated heterocyclic ring having 1-4 heteroatoms independently selected from N, O, or S, wherein R₁₂ is optionally substituted with 1-2 of C₁₋₆ alkyl to generate the compound of Formula III.

78-106. (canceled)

107. A method of preparing a compound of Formula IV 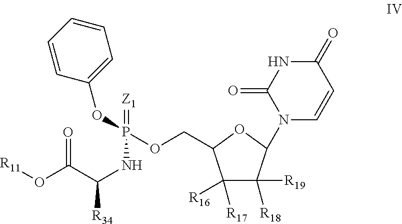
or a pharmaceutically acceptable salt thereof, having a diastereomeric purity of about 75% or greater, wherein
Z₁ is O or S;
R₃₄ is C₁₋₆ alkyl, halo-C₁₋₆ alkyl, C₃₋₈ cycloalkyl, aryl, or aryl(C₁₋₆ alkyl);
R₁₁ is hydrogen, C₁₋₆ alkyl, C₃₋₈ cycloalkyl, aryl, aryl(C₁₋₆ alkyl), or halo-C₁₋₆ alkyl;
Each of R₁₆, R₁₇, R₁₈, and R₁₉ is independently selected from hydrogen, —OH, halogen, azido, cyano, an optionally substituted C₁₋₆ alkyl, —OR₂₀ or —OC(O)R₂₁, or
R₁₇ and R₁₈ are both oxygen atoms that are linked together by —(CR₂₁R₂₂)— or by a carbonyl group; and
Each of R₂₀, R₂₁, and R₂₂ is independently selected from hydrogen, optionally substituted C₁₋₆ alkyl or optionally substituted C₃₋₆ cycloalkyl;
comprising the step of:
ic) reacting a compound of Formula A-2 with a compound of Formula B-1C 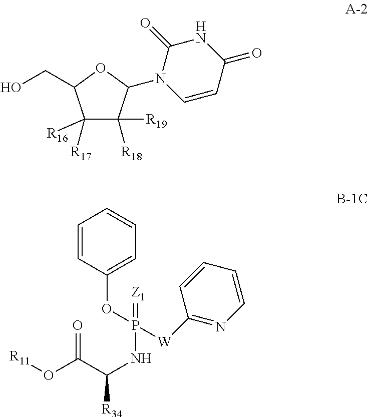
in the presence of an acid or a metal salt to generate the compound of Formula IV.

108-137. (canceled)

138. A compound of Formula B-1B: 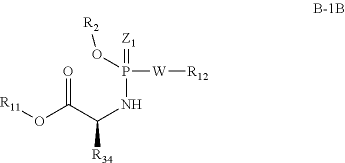
wherein
Z₁ is S or O;
R₂ is optionally substituted aryl or optionally substituted heteroaryl;
W is a bond, —S—, or —O—; and
R₁₂ is a 5-10-membered mono- or bicyclic saturated, partially unsaturated, a fully unsaturated heterocyclic ring having 1-4 heteroatoms independently selected from N, O, or S, optionally substituted with 1-3 of R₁₃, wherein R₁₃ is oxo or an optionally substituted C₁₋₆ alkyl; or 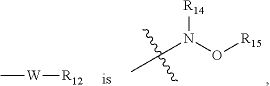
wherein each of R₁₄ and R₁₅ is independently C₁₋₆ alkyl, cycloalkyl, or heteroalkyl, or
R₁₄ and R₁₅, taken together with the heteroatoms to which they are attached, form a 6-10-membered heterocyclic ring optionally substituted with 1-3 of R₁₃;
R₃₄ is C₁₋₆ alkyl, halo-C₁ alkyl, C₃₋₈ cycloalkyl, aryl, or aryl(C₁₋₆ alkyl); and
R₁₁ is hydrogen, C₁₋₆ alkyl, C₃₋₈ cycloalkyl, aryl, aryl(C₁₋₆ alkyl), or halo-C₁₋₆ alkyl.

139-151. (canceled)

152. A method of preparing a compound of Formula V 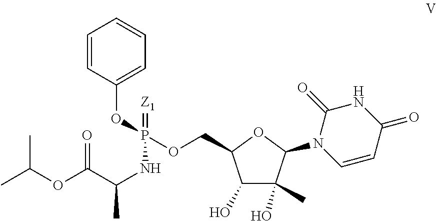
or a pharmaceutically acceptable salt thereof, having a diastereomeric purity of about 75% or greater; wherein Z₁ is S or O, comprising the step of:
id) reacting a compound of Formula A-3 and a compound of Formula B-4B1 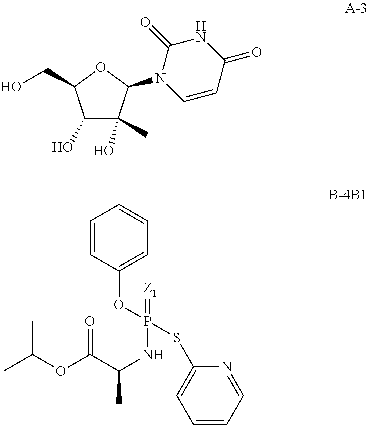
in the presence of an acid or a metal salt to generate the compound of Formula V.

153-187. (canceled)
